(19) United States
(12) Patent Application Publication
(10) Pub. No.: US 2025/0009731 A1
(43) Pub. Date: Jan. 9, 2025

(54) TOLL-LIKE RECEPTOR THERAPY COMBINATIONS WITH CBL-B INHIBITOR COMPOUNDS

(71) Applicant: NURIX THERAPEUTICS, INC., San Francisco, CA (US)

(72) Inventors: Marilena GALLOTTA, San Francisco, CA (US); Cristiana GUIDUCCI, San Francisco, CA (US)

(21) Appl. No.: 18/708,175

(22) PCT Filed: Nov. 7, 2022
(86) PCT No.: PCT/US2022/049171

§ 371 Date: May 7, 2024

Related U.S. Application Data
(60) Provisional application No. 63/277,122, filed on Nov. 8, 2021.
(60) Provisional application No. 63/290,589, filed on Dec. 16, 2021.

(51) Int. Cl. A61K 31/454; A61K 31/4745; A61K 31/7125; A61K 31/713; A61P 35/00

(52) U.S. Cl. CPC A61K 31/454; A61K 31/4745; A61K 31/7125; A61K 31/713; A61P 35/00

(57) ABSTRACT

The present disclosure relates to combination therapies with Cbl-b inhibitor compounds, and compositions and kits comprising combinations with the Cbl-b compounds. Also provided are methods of using the combinations with Cbl-b compounds and compositions thereof, such as in therapeutic methods.

### CROSS REFERENCE

This application claims the benefit of U.S. provisional application nos. 63/290,589, filed Dec. 16, 2021, and 63/277,122, filed Nov. 8, 2021, the contents of each of which are hereby incorporated by reference in their entireties.

### FIELD

Provided herein are combination therapies with Cbl-b inhibitor compounds, compositions for administering the same, including pharmaceutical compositions, and kits for administering the same. The methods and compositions are useful for the treatment and prevention of cell proliferation and cancer.

### BACKGROUND

Casitas B-lineage lymphoma proto-oncogene-b (Cbl-b) is an E3 ubiquitin ligase that negatively regulates T-cell activation. Wallner et al., Clin. Dev. Immunol., 2012: 692639. Certain Cbl-b inhibitor compounds have shown promise for several potential immunotherapy applications through enhancing T-cell mediated anti-tumor activity by lowering the activation threshold of T-cells in a suppressive tumor microenvironment where Cbl-b plays a role in the downregulation of T-cells. Because of the mechanism of Cbl-b inhibitors, they have the potential to enhance the efficacy of other cancer therapeutics, particularly those that stimulate an immune response.

Viral and bacterial mediated immune responses typically are initiated by the binding of certain oligonucleotides to toll-like receptors (TLRs) that are expressed by host immune cells such as dendritic cells, macrophages, natural killer cells, and antigen presenting cells. Du et al., 2000, Eur. Cytokine Netw. 11(3):362-71. The binding of a TLR agonist triggers the host's innate immune response as well as activates the adaptive response. Marshak-Rothstein, 2006, Nat. Rev. Immunol. 6(11):823-835. Because of the immunostimulatory effect of TLR agonists, synthetic TLR agonists have been studied for the activation of the immune system for the treatment of human diseases including cancer. Ilvesaro et al., 2008, Mol. Cancer Res. 6(10):1534-43.

### SUMMARY

Provided herein are Cbl-b inhibitor compounds for use in combination with a second therapeutic agent that modulates cell division or cell differentiation activity. As demonstrated in the Examples herein, the combination of a Cbl-b inhibitor compound with the second therapeutic agent yields substantially increased efficacy against solid tumors and/or increased survival in an in vivo model. Cbl-b inhibitor compounds are described in detail herein, as are their pharmaceutical compositions, and methods for making them. In particular embodiments, the second therapeutic agent is an immunostimulatory sequence(s). In particular embodiments, the second therapeutic agent is a CpG-C type oligonucleotide.

In one aspect, provided herein are methods of using the Cbl-b inhibitor compounds in combination with a second therapeutic agent that modulates cell division or cell differentiation activity. In certain embodiments, the second therapeutic agent is a TLR7 agonist. In certain embodiments, the second therapeutic agent is a TLR9 agonist. In certain embodiments, the methods are methods of treatment. In certain embodiments, the combination is used to treat a disease or condition. In certain embodiments, the disease or condition is a cancer.

In another aspect, provided are kits or compositions comprising a Cbl-b inhibitor compound and a second therapeutic agent that modulates cell division or cell differentiation activity. In certain embodiments, the Cbl-b compounds and the second therapeutic agent(s) are in separate pharmaceutical compositions. In certain embodiments, the Cbl-b compounds and the second therapeutic agent(s) are administered separately. In certain embodiments, the Cbl-b compounds and the second therapeutic agent(s) are administered cyclically. In certain embodiments, the compositions are pharmaceutical compositions. Any suitable pharmaceutical composition may be used. In certain embodiments, the pharmaceutical composition for the Cbl-b compound is a composition for oral administration. In certain embodiments, the pharmaceutical composition for the second therapeutic agent is a composition for parenteral administration. In a particular embodiment, the second therapeutic agent is a TLR7 agonist. In a particular embodiment, the second therapeutic agent is a TLR9 agonist.

The methods, kits, and compositions are useful for inhibiting cell proliferation. In certain embodiments, the methods, kits, and compositions are useful for treating a disease or disorder. In certain embodiments, the disease or disorder is a cancer. In certain embodiments, provided herein are the combinations, kits, and compositions for use in therapy. In certain embodiments, provided herein are the combinations, kits, and compositions for use in the treatment of a cancer.

### BRIEF DESCRIPTION OF THE DRAWING

FIG. 1 provides effects of Compound 23 on total primary human T-cells.

FIG. 2 provides tumor volume in mice bearing tumors 25 days following administration of vehicle or Compound 23.

FIG. 3 provides the effect of Compound 23 on survival of mice bearing 4T1 primary tumors, which are syngenic triple negative mammary carcinoma models.

FIG. 4A provides the effects of orally administered Compound 23 on tumor infiltrating lymphocytes (TIL) after 4 or 19 daily doses. FIG. 4B provides the effects of orally administered Compound 23 on gene expression immune related pathway scores in CT26 tumor tissue after 4 doses. FIG. 4C provides the effects of orally administered Compound 23 on gene expression immune related pathway scores in CT26 tumor tissue after 19 doses.

FIG. 5 provides antitumor efficacy in mice bearing CT26 tumors following oral Compound 23 at 30 mg/kg in the presence of depleting antibodies for CD4+ cells, CD8+ cells, or NK cells (anti-asialo-GM1).

FIG. 6, panels 6A-6D, provide the effect of Compound 23 treatment on CD8+ T cell immune phenotype, both in tumor and blood samples from treated 4T1-tumor-bearing mice.

FIG. 7, panels 7A-7I, provide the effect of Compound 23 treatment on the density and phenotype of tumor-infiltrating leukocytes from treated CT2 v6-tumor-bearing mice.

FIG. 8, panels 8A-8F, provide the strong correlation of antitumor activity of Compound 23 with increased levels of circulating T- and NK cells, CD8+ T-cells, and activated CD8+ T-cells, and decreased levels of circulating myeloid cells (CD11b+) in the blood of treated CT26-tumor-bearing mice.

FIG. 9 provides long-term survival of mice bearing CT26 tumors after dosing with vehicle, Compound 23, CpG oligonucleotide C792, and a combination of Compound 23 and C792.

### DETAILED DESCRIPTION

### 1. Definitions

Unless otherwise defined, all terms of art, notations, and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, for example, the widely utilized molecular cloning methodologies described in Sambrook et al., Molecular Cloning: A Laboratory Manual 2nd ed. (1989) Cold Spring Harbor Laboratory Press, Cold Spring Harbor, NY. As appropriate, procedures involving the use of commercially available kits and reagents are generally carried out in accordance with manufacturer-defined protocols and conditions unless otherwise noted.

It is understood that aspects and embodiments described herein as “comprising” include “consisting of” and “consisting essentially of” embodiments.

As used herein and in the appended claims, the singular forms “a,” “an,” and “the” include plural referents unless otherwise indicated or clear from context. For example, “an” excipient includes one or more excipients.

As used herein, references to “about” a particular value encompasses from 90% to 110% of that value. For instance, “about 50 billion cells” refers to 45 to 55 billion cells, and includes 50 billion cells. For instance, a temperature of “about 100 degrees” refers to a temperature of about 90 degrees to about 110 degrees.

When numerical ranges of compounds are given, all compounds within those numerical limits designated “a” and “b” are included, unless expressly excluded. For example, reference to compounds 9-13 refers to compounds 9, 10, 11, 12, and 13. By way of further example, reference to compounds 9-13 also refers to compounds 9, 9a, 10, 10a, 10b, 11, 12, and 13.

The term “3′,” as used herein, generally refers to a region or position in a polynucleotide or oligonucleotide 3′ (i.e., downstream) from another region or position in the same polynucleotide or oligonucleotide. The term “3′ end” refers to the 3′ terminus of the polynucleotide.

The term “5′,” as used herein, generally refers to a region or position in a polynucleotide or oligonucleotide 5′ (i.e., upstream) from another region or position in the same polynucleotide or oligonucleotide. The term “5′ end” refers to the 5′ terminus of the polynucleotide.

As used herein, a region, portion, or sequence which is “adjacent” to another sequence directly abuts that other region, portion, or sequence. For example, an additional polynucleotide sequence (e.g., a TCG trinucleotide) which is adjacent to a particular portion of an immunomodulatory polynucleotide directly abuts that region.

The terms “antisense” and “antisense sequence,” as used herein, refer to a non-coding strand of a polynucleotide having a sequence complementary to the coding strand of mRNA. In certain embodiments, the polynucleotides of the present disclosure are not antisense sequences, or RNAi molecules (miRNA and siRNA). That is in certain embodiments, the polynucleotides of the present disclosure do not have significant homology (or complementarity) to transcripts (or genes) of the mammalian subjects in which they will be used. For instance, a polynucleotide of the present disclosure for modulating an immune response in a human subject is less than 80% identical over its length to nucleic acid sequences of the human genome (e.g., a polynucleotide that is 50 nucleotides in length would share no more than 40 of the 50 bases with a human transcript). That is, in certain embodiments, the polynucleotides are less than 80%, 75%, 70%, 65%, 60%, 55%, 50%, 45%, 40%, 35%, 30%, 25%, or 20% identical to nucleic acid sequences of mammalian subjects (e.g., such as humans, nonhuman primates, farm animals, dogs, cats, rabbits, rats, mice, etc.) in which they are to be used.

The term “Cbl-b,” as used herein, refers to a Cbl-b protein. The term also includes naturally occurring variants of Cbl-b, including splice variants or allelic variants. The term also includes non-naturally occurring variants of Cbl-b, such as a recombinant Cbl-b protein or truncated variants thereof, which generally preserve the binding ability of naturally occurring Cbl-b or naturally occurring variants of Cbl-b (e.g., the ability to bind to an E2 enzyme).

The terms “CpG” and “CG” are used interchangeably herein to refer to a cytosine (C) and guanine (G) separated by a phosphate (“p”). These terms refer to a linear sequence as opposed to base-pairing of cytosine and guanine. The polynucleotides of the present disclosure contain at least one unmethylated CpG dinucleotide. That is the cytosine in the CpG dinucleotide is not methylated (i.e., is not 5-methylcytosine).

“CpG-C type oligonucleotides,” as used herein, are oligonucleotides from 12 to 100 bases in length, which have one or more 5′-TCG trinucleotides wherein the 5′-T is positioned 0, 1, 2, or 3 bases from the 5′-end of the oligonucleotide, and at least one palindromic sequence of at least 8 bases in length comprising one or more unmethylated CG dinucleotides. The one or more 5′-TCG trinucleotide sequence may be separated from the 5′-end of the palindromic sequence by 0, 1, or 2 bases or the palindromic sequence may contain all or part of the one or more 5′-TCG trinucleotide sequence. In one embodiment, the oligonucleotide is an oligodeoxynucleotide (ODN). In one embodiment, the oligonucleotide is a 2′-oligodeoxynucleotide. CpG-C ODNs have the ability to stimulate B cells, induce plasmacytoid dendritic cell (PDC) maturation and cause secretion of high levels of type I interferons (e.g., IFN-α, IFN-γ, etc.). In some embodiments, the CpG-C ODNs are 12 to 100 bases in length, 12 to 50 bases in length, 12 to 40 bases in length, or 12-30 bases in length.

The term “immunomodulatory” or “modulating an immune response,” as used herein, includes immunostimulatory as well as immunosuppressive effects. Immunomodulation is primarily a qualitative alteration in an overall immune response, although quantitative changes may also occur in conjunction with immunomodulation. An immune response that is immunomodulated according to this disclosure is one that is shifted towards a “Th1-type” immune response, as opposed to a “Th2-type” immune response. Th1-type responses are typically considered cellular immune system (e.g., cytotoxic lymphocytes) responses, while Th2-type responses are generally “humoral,” or antibody-based. Th1-type immune responses are normally characterized by “delayed-type hypersensitivity” reactions to an antigen and can be detected at the biochemical level by increased levels of Th1-associated cytokines such as IFN-γ, IFN-α, IL-2, IL-12, and TNF-β, as well as IL-6, although IL-6 may also be associated with Th2-type responses as well. Th1-type immune responses are generally associated with the production of cytotoxic lymphocytes (CTLs) and low levels or transient production of antibody. Th2-type immune responses are generally associated with higher levels of antibody production, including IgE production, an absence of or minimal CTL production, as well as expression of Th2-associated cytokines such as IL-4. Accordingly, immunomodulation in accordance with this disclosure may be recognized by, for example, an increase in IFN-γ and/or IFN-α and/or a decrease in IgE production in an individual treated in accordance with the methods of this disclosure as compared to the absence of treatment.

The terms “immunostimulatory sequence” and “ISS,” as used herein, refer to a nucleic acid sequence that stimulates a measurable immune response (e.g., measured in vitro, in vivo, and/or ex vivo). For the purpose of the present disclosure, the term ISS refers to a nucleic acid sequence comprising an unmethylated CG dinucleotide. Examples of measurable immune responses include, but are not limited to, antigen-specific antibody production, cytokine secretion, lymphocyte activation, and lymphocyte proliferation.

“Inhibition” of a response or parameter, as used herein, includes blocking and/or suppressing that response or parameter when compared to the same conditions otherwise except for a parameter of interest, or alternatively, as compared to another condition.

“Monoclonal antibody” or “mAb” or “Mab,” as used herein, refers to a population of substantially homogeneous antibodies (i.e., the antibody molecules comprising the population are identical in amino acid sequence except for possible naturally occurring mutations that may be present in minor amounts). In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and it is not to be construed as requiring production of the antibody by any particular method. For example, the monoclonal antibodies to be used in accordance with this disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides, or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G), or derivatives thereof) or pyrimidine (thymine (T), cytosine (C), or uracil (U), or derivatives thereof) base bonded to a sugar. The four nucleoside units (or bases) in DNA are called deoxyadenosine, deoxyguanosine, deoxythymidine, and deoxycytidine. A nucleotide is a phosphate ester of a nucleoside.

The term “palindromic sequence” or “palindrome,” as used herein, refers to a nucleic acid sequence that is an inverted repeat, e.g., ABCDD′C′B′A′, where the bases, e.g., A and A′, B and B′, C and C′, D and D′, are capable of forming the Watson-Crick base pairs. Such sequences may be single-stranded or may form double-stranded structures or may form hairpin loop structures under some conditions. For example, as used herein, “an 8 base palindrome” or “8-base palindrome” refers to a nucleic acid sequence in which the palindromic sequence is 8 bases in length, such as ABCDD′C′B′A′. A palindromic sequence may be part of a polynucleotide which also contains non-palindromic sequences. A polynucleotide may contain one or more palindromic sequence portions and one or more non-palindromic sequence portions. Alternatively, a polynucleotide sequence may be entirely palindromic. In a polynucleotide with more than one palindromic sequence portion, the palindromic sequence portions may overlap with each other or the palindromic sequence portions may not overlap with each other.

“Stimulation” of a response or parameter, as used herein, includes eliciting and/or enhancing that response or parameter when compared to the same conditions otherwise except for a parameter of interest, or alternatively, as compared to another condition (e.g., increase in TLR-signaling in the presence of a TLR agonist as compared to the absence of the TLR agonist). For example, “stimulation” of an immune response means an increase in the response.

As used herein, the term “T-cell dysfunction” refers to a state of reduced immune responsiveness to antigenic stimulation. The term “T-cell dysfunction” includes common elements of both T-cell exhaustion and/or T-cell anergy in which antigen recognition may occur, but the ensuing immune response is ineffective to control tumor growth. The term “T-cell dysfunction” also includes being refractory or unresponsive to antigen recognition, such as, impaired capacity to translate antigen recognition to downstream T-cell effector functions, such as proliferation, cytokine production, and/or target cell killing.

The term “T-cell anergy,” as used herein, refers to the state of unresponsiveness to antigen stimulation resulting from incomplete or insufficient signals delivered through the T-cell receptor. “T-cell anergy” can also result upon stimulation with antigen in the absence of co-stimulation, resulting in the cell becoming refractory to subsequent activation by the antigen even in the context of co-stimulation.

The term “T-cell exhaustion,” as used herein, refers to a state of T-cell dysfunction that arises from sustained TCR signaling that can occur during cancer. It is distinguished from anergy in that it arises not through incomplete or deficient signaling, but from sustained signaling. It is defined by poor effector function, sustained expression of inhibitory receptors, and a transcriptional state distinct from that of functional effector or memory T-cell.

As used herein, a “T-cell dysfunction disorder” is a disorder or condition characterized by decreased responsiveness of T-cells to antigenic stimulation. Decreased responsiveness may result in ineffective control of a tumor. In certain embodiments, the term “T-cell dysfunction disorder” encompasses cancer such as a hematologic cancer or a non-hematologic cancer. In certain embodiments, a “T-cell dysfunctional disorder” is one in which T-cells are anergic or have decreased ability to secrete cytokines, proliferate, or execute cytolytic activity.

As used herein, “enhancing T-cell function” means to induce, cause, or stimulate a T-cell to have a sustained or amplified biological function, or renew or reactivate exhausted or inactive T-cells. Examples of enhanced T-cell function include increased T-cell activation (e.g., increased cytokine production, increased expression of T-cell activation markers, etc.), increased T-cell proliferation, decreased T-cell exhaustion, and/or decreased T-cell tolerance relative to the state of the T-cells before treatment with a Cbl-b inhibitor compound. Methods of measuring enhancement of T-cell function are known in the art.

“TLR7,” as used herein, refers to toll-like receptor 7, a protein that in humans is encoded by the TLR7 gene that detects single stranded RNA. as a member of the toll-like receptor family, alternative names for TLR7 include TLR7-like and IMD74. Human TLR7 amino acid sequences can be found at NM_016562 (mRNA) and NP_057646 (protein), each incorporated by reference in its entirety.

The term “TLR7 agonist,” as used herein, refers to a molecule that binds to TLR7 and results in signal transduction from the interaction of the agonist and TLR7.

As used herein, “TLR9” refers to toll-like receptor 9, a protein that in humans is encoded by the TLR9 gene that binds to bacterial and viral DNA. As a member of the toll like receptor family, alternate names for TLR9 include CD289. Human TLR9 amino acid sequences can be found at NM_138699 and NM_017442 (mRNA) and NP_059138, each incorporated by reference in its entirety.

The term “TLR9 agonist,” as used herein, refers to a molecule that binds to TLR9 and results in signal transduction from the interaction of the agonist and TLR9.

“Alkyl” as used herein refers to a saturated linear (i.e., unbranched) or branched univalent hydrocarbon chain or combinations thereof. Particular alkyl groups are those having a designated number of carbon atoms, for example, an alkyl group having one to twenty carbon atoms (a “C₁-C₂₀ alkyl”), having one to ten carbon atoms (a “C₁-C₁₀” alkyl), having one to eight carbon atoms (a “C₁-C₈ alkyl”), having one to six carbon atoms (a “C₁-C₆ alkyl”), having two to six carbon atoms (a “C₂-C₆ alkyl”), or having one to four carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, and homologs and isomers of, for example, n-pentyl, n-hexyl, n-heptyl, n-octyl, and the like.

“Alkenyl” as used herein refers to an unsaturated linear (i.e., unbranched) or branched univalent hydrocarbon chain or combinations thereof, having at least one site of olefinic unsaturation (i.e., having at least one moiety of the formula C═C). Particular alkenyl groups are those having a designated number of carbon atoms, for example, an alkenyl group having 2 to 20 carbon atoms (a “C₂-C₂₀ alkenyl”), having 2 to 10 carbon atoms (a “C₂-C₁₀” alkenyl), having 2 to 8 carbon atoms (a “C₂-C₈ alkenyl”), having 2 to 6 carbon atoms (a “C₂-C₆ alkenyl”), or having 2 to 4 carbon atoms (a “C₂-C₄ alkenyl”). The alkenyl group may be in “cis-” or “trans-” configurations or, alternatively, in “E-” or “Z-” configurations. Examples of alkenyl groups include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

“Alkynyl” as used herein refers to an unsaturated linear (i.e., unbranched) or branched univalent hydrocarbon chain or combinations thereof, having at least one site of acetylenic unsaturation (i.e., having at least one moiety of the formula C≡C). Particular alkynyl groups are those having a designated number of carbon atoms, for example, an alkynyl group having 2 to 20 carbon atoms (a “C₂-C₂₀ alkynyl”), having 2 to 10 carbon atoms (a “C₂-C₁₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl groups include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

“Alkylene” as used herein refers to the same residues as alkyl, but having bivalency or are divalent. Particular alkylene groups are those having one to six carbon atoms (a “C₁-C₆ alkylene”), one to five carbon atoms (a “C₁-C₅ alkylene”), one to four carbon atoms (a “C₁-C₄ alkylene”), or one to three carbon atoms (a “C₁-C₃ alkylene”). Examples of alkylene groups include, but are not limited to, groups such as methylene (—CH₂—), —CH₂CH₂—, —CH₂CH₂CH₂—, —CH₂CH₂CH₂CH₂—, and the like.

“Cycloalkyl” as used herein refers to non-aromatic, saturated or unsaturated, cyclic, univalent hydrocarbon structures. Particular cycloalkyl groups are those having a designated number of annular (i.e., ring) carbon atoms, for example, a cycloalkyl group having from 3 to 12 annular carbon atoms (a “C₃-C₁₂ cycloalkyl”). A particular cycloalkyl is a cyclic hydrocarbon having from 3 to 8 annular carbon atoms (a “C₃-C₈ cycloalkyl”) or having 3 to 6 annular carbon atoms (a “C₃-C₆ cycloalkyl”). Cycloalkyl can consist of one ring, such as cyclohexyl, or multiple rings, such as adamantyl, but excludes aryl (i.e., aromatic) groups. A cycloalkyl comprising more than one ring may be fused, spiro, or bridged, or combinations thereof. Examples of cycloalkyl groups include, but are not limited to, cyclopropyl

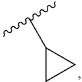 [SMILES: *C1CC1]

cyclobutyl

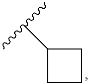 [SMILES: *C1CCC1]

cyclopentyl

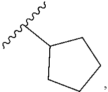 [SMILES: *C1CCCC1]

cyclohexyl

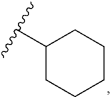 [SMILES: *C1CCCCC1]

1-cyclohexenyl

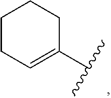 [SMILES: *C1=CCCCC1]

3-cyclohexenyl

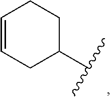 [SMILES: *C1CC=CCC1]

cycloheptyl

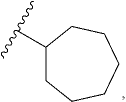 [SMILES: *C1CCCCCC1]

norbornyl, and the like.

“Cycloalkylene” as used herein refers to the same residues as cycloalkyl, but having bivalency or are divalent. Particular cycloalkylene groups are those having 3 to 12 annular carbon atoms (a “C₃-C₁₂ cycloalkylene”), having from 3 to 8 annular carbon atoms (a “C₃-C₈ cycloalkylene”), or having 3 to 6 annular carbon atoms (a “C₃-C₆ cycloalkylene”). Examples of cycloalkylene groups include, but are not limited to, cyclopropylene

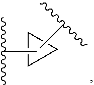

cyclobutylene

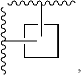

cyclopentylene

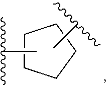

cyclohexylene

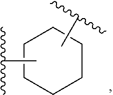

cycloheptylene

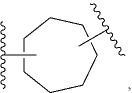

norbornylene, and the like.

“Aryl” as used herein refers to an aromatic carbocyclic group having a single ring (e.g., phenyl), or multiple condensed rings (e.g., naphthyl or anthryl) where one or more of the condensed rings may not be aromatic. Particular aryl groups are those having from 6 to 14 annular (i.e., ring) carbon atoms (a “C₆-C₁₄ aryl”). An aryl group having more than one ring where at least one ring is non-aromatic may be connected to the parent structure at either an aromatic ring position or at a non-aromatic ring position. In one variation, an aryl group having more than one ring where at least one ring is non-aromatic is connected to the parent structure at an aromatic ring position. Examples of aryls include, but are not limited to, groups such as phenyl, naphthyl, 1,2,3,4-tetrahydronaphthalen-6-yl

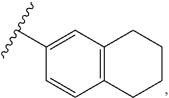 [SMILES: *c1ccc2c(c1)CCCC2]

and the like.

“Carbocyclyl” or “carbocyclic” refers to an aromatic or non-aromatic univalent cyclic group in which all of the ring members are carbon atoms, such as cyclohexyl, phenyl, 1,2-dihydronaphthyl, and the like.

“Arylene” as used herein refers to the same residues as aryl, but having bivalency or are divalent. Particular arylene groups are those having from 6 to 14 annular carbon atoms (a “C₆-C₁₄ arylene”). Examples of arylene include, but are not limited to, phenylene

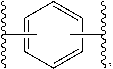

naphthylene

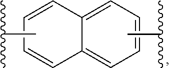

and the like.

“Heteroaryl” as used herein refers to an unsaturated aromatic cyclic group having from one to fourteen annular carbon atoms and at least one annular heteroatom, including but not limited to, nitrogen (N), oxygen (O), and sulfur (S). A heteroaryl group may have a single ring (e.g., pyridyl or imidazolyl) or multiple condensed rings (e.g., indolizinyl, indolyl, or quinolinyl) where at least one of the condensed rings is aromatic. Particular heteroaryl groups are 5- to 14-membered rings having one to twelve annular carbon atoms and one to six annular heteroatoms independently selected from the group consisting of nitrogen (N), oxygen (O), and sulfur (S) (e.g., a “5- to 14-membered heteroaryl”); 5- to 10-membered rings having one to eight annular carbon atoms and one to four annular heteroatoms independently selected from the group consisting of nitrogen, oxygen, and sulfur (e.g., a “5- to 10-membered heteroaryl”); or 5-, 6-, or 7-membered rings having one to five annular carbon atoms and one to four annular heteroatoms independently selected from the group consisting of nitrogen, oxygen, and sulfur (a “5- to 7-membered heteroaryl”). In one variation, heteroaryl includes monocyclic aromatic 5-, 6-, or 7-membered rings having from one to six annular carbon atoms and one to four annular heteroatoms independently selected from the group consisting of nitrogen, oxygen, and sulfur. In another variation, heteroaryl includes polycyclic aromatic rings having from one to twelve annular carbon atoms and one to six annular heteroatoms independently selected from the group consisting of nitrogen, oxygen, and sulfur. A heteroaryl group having more than one ring where at least one ring is non-aromatic may be connected to the parent structure at either an aromatic ring position or at a non-aromatic ring position. Examples of heteroaryl include, but are not limited to, groups such as pyridyl, benzimidazolyl, benzotriazolyl, benzo[b]thienyl, quinolinyl, indolyl, benzothiazolyl, and the like. “Heteroaryl” also includes moieties such as

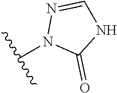 [SMILES: *n1nc[nH]c1=O]

(2,4-dihydro-3H-1,2,4-triazol-3-one-2-yl), which has the aromatic tautomeric structure

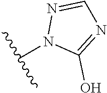 [SMILES: *n1ncnc1O]

(1H-1,2,4-triazol-5-ol-1-yl).

“Heterocyclyl” and “heterocyclic groups” as used herein refer to non-aromatic saturated or partially unsaturated cyclic groups having the number of atoms and heteroatoms as specified, or if no number of atoms or heteroatoms is specified, having at least three annular atoms, from one to fourteen annular carbon atoms, and at least one annular heteroatom, including, but not limited to, heteroatoms such as nitrogen, oxygen, and sulfur. A heterocyclic group may have a single ring (e.g., tetrahydrothiophen-yl, oxazolidinyl) or multiple condensed rings (e.g., decahydroquinolinyl, octahydrobenzo[d]oxazolyl). Multiple condensed rings include, but are not limited to, bicyclic, tricyclic, and quadracylic rings, as well as bridged or spirocyclic ring systems. Examples of heterocyclic groups include, but are not limited to, aziridinyl, azetidinyl, pyrrolidinyl, piperidinyl, oxiranyl, oxetanyl, tetrahydrofuranyl, tetrahydropyranyl, oxazolidinyl, piperazinyl, morpholinyl, dioxanyl, 3,6-dihydro-2H-pyranyl, 2,3-dihydro-1H-imidazolyl, and the like.

“Heteroarylene” as used herein refers to the same residues as heteroaryl, but having bivalency or are divalent. Particular heteroarylene groups are 5- to 14-membered rings having one to twelve annular carbon atoms and one to six annular heteroatoms independently selected from the group consisting of nitrogen, oxygen, and sulfur (a “5- to 14-membered heteroarylene”); 5- to 10-membered rings having one to eight annular carbon atoms and one to four annular heteroatoms independently selected from the group consisting of nitrogen, oxygen, and sulfur (a “5- to 10-membered heteroarylene”); or 5-, 6-, or 7-membered rings having one to five annular carbon atoms and one to four annular heteroatoms independently selected from the group consisting of nitrogen, oxygen, and sulfur (a “5- to 7-membered heteroarylene”). Examples of heteroarylene include, but are not limited to, groups such as pyridylene, benzimidazolylene, benzotriazolylene, benzo[b]thienylene, quinolinylene, indolylene, benzothiazolylene, and the like.

“Halo” or “halogen,” as used herein, refers to elements of the Group 17 series having atomic number 9 to 85. Halo groups include fluoro (F), chloro (Cl), bromo (Br), and iodo (I).

“Haloalkyl,” “haloalkylene,” “haloaryl,” “haloarylene,” “haloheteroaryl,” and similar terms, as used herein, refer to a moiety substituted with at least one halo group. Where a haloalkyl moiety or other halo-substituted moiety is substituted with more than one halogen, it may be referred to by using a prefix corresponding to the number of halogen moieties attached. For example, dihaloaryl, dihaloalkyl, trihaloaryl, trihaloalkyl, etc., refer to aryl and alkyl substituted with two (“di”) or three (“tri”) halo groups, which may be, but are not necessarily, the same halo; thus, for example, the haloaryl group 4-chloro-3-fluorophenyl is within the scope of dihaloaryl. The subset of haloalkyl groups in which each hydrogen (H) of an alkyl group is replaced with a halo group is referred to as a “perhaloalkyl.” A particular perhaloalkyl group is trifluoroalkyl (—CF₃). Similarly, “perhaloalkoxy” refers to an alkoxy group in which a halogen takes the place of each hydrogen (H) in the hydrocarbon making up the alkyl moiety of the alkoxy group. An example of a perhaloalkoxy group is trifluoromethoxy (—OCF₃). “Haloalkyl” includes monohaloalkyl, dihaloalkyl, trihaloalkyl, perhaloalkyl, and any other number of halo substituents possible on an alkyl group; and similarly for other groups such as haloalkylene, haloaryl, haloarylene, haloheteroaryl, etc.

“Amino,” as used herein, refers to the group —NH₂.

“Oxo,” as used herein, refers to the group ═O, that is, an oxygen atom doubly bonded to carbon (e.g., a carbonyl) or another chemical element.

“Optionally substituted,” as used herein and unless otherwise specified, means that a group is unsubstituted or substituted by one or more (e.g., 1, 2, 3, 4, or 5) of the substituents listed for that group, in which the substituents may be the same or different. In one embodiment, an optionally substituted group is unsubstituted. In one embodiment, an optionally substituted group has one substituent. In another embodiment, an optionally substituted group has two substituents. In another embodiment, an optionally substituted group has three substituents. In another embodiment, an optionally substituted group has four substituents. In certain embodiments, an optionally substituted group has one to two, one to three, one to four, or one to five substituents. When multiple substituents are present, each substituent is independently chosen unless indicated otherwise. For example, each (C₁-C₄ alkyl) substituent on the group —N(C₁-C₄ alkyl)(C₁-C₄ alkyl) can be selected independently from the other, so as to generate groups such as —N(CH₃)(CH₂CH₃), etc.

The term “substituted,” as used herein to modify a specified group or radical, can also mean that one or more hydrogen atoms (H) of the specified group or radical are each, independently of one another, replaced with the same or different substituent groups as defined herein. In certain embodiments, a group that is substituted has one, two, three, or four substituents, one, two, or three, substituents, one or two substituents, or one substituent.

Substituents can be attached to any chemically possible location on the specified group or radical, unless indicated otherwise. Thus, in one embodiment, —C₁-C₈ alkyl-OH includes, for example, —CH₂CH₂OH, —CH(OH)—CH₃, —CH₂C(OH)(CH₃)₂, and the like. By way of further example, in one embodiment, —C₁-C₆ alkyl-OH includes, for example, —CH₂CH₂OH, —CH(OH)—CH₃, —CH₂C(OH)(CH₃)₂, and the like. By way of further example, in one embodiment, —C₁-C₆ alkyl-CN includes, for example, —CH₂CH₂CN, —CH(CN)—CH₃, —CH₂C(CN)(CH₃)₂, and the like.

Unless a specific isotope of an element is indicated in a formula, this disclosure includes all isotopologues of the compounds disclosed herein for example, deuterated derivatives of the compounds (where H can be ²H, i.e., deuterium (D)). Deuterated compounds may provide favorable changes in pharmacokinetic (ADME) properties. Isotopologues can have isotopic replacements at any or at all locations in a structure, or they can have atoms present in natural abundance at any or all locations in a structure.

A “small molecule” as used herein refers to a compound of 1,000 daltons or less in molecular weight.

Hydrogen atoms can also be replaced with close bioisosteres, such as fluorine, provided that such replacements result in stable compounds.

This disclosure also includes any or all of the stereochemical forms, including any enantiomeric or diastereomeric forms of the compounds described herein, and cis-/trans- or E-/Z-isomers. Unless stereochemistry is explicitly indicated in a chemical structure or name, the structure or name is intended to embrace all possible stereoisomers of a compound depicted. In addition, where a specific stereochemical form is depicted, it is understood that all other stereochemical forms are also described and embraced by this disclosure, as well as the general non-stereospecific form and mixtures of the disclosed compounds in any ratio, including mixtures of two or more stereochemical forms of a disclosed compound in any ratio, such that racemic, non-racemic, enantioenriched, and scalemic mixtures of a compound are embraced. Compositions comprising a disclosed compound also are intended, such as a composition of a substantially pure compound, including a specific stereochemical form thereof. Compositions comprising a mixture of disclosed compounds in any ratio also are embraced by this disclosure, including compositions comprising mixtures of two or more stereochemical forms of a disclosed compound in any ratio, such that racemic, non-racemic, enantioenriched, and scalemic mixtures of a compound are embraced by this disclosure. If stereochemistry is explicitly indicated for one portion or portions of a molecule, but not for another portion or portions of a molecule, the structure is intended to embrace all possible stereoisomers for the portion or portions where stereochemistry is not explicitly indicated.

This disclosure also embraces any and all tautomeric forms of the compounds described herein.

This disclosure is intended to embrace all salts of the compounds described herein, as well as methods of using such salts of the compounds. In one embodiment, the salts of the compounds comprise pharmaceutically acceptable salts. Pharmaceutically acceptable salts are those salts that can be administered as drugs or pharmaceuticals to humans and/or animals and that, upon administration, retain at least some of the biological activity of the free compound (i.e., neutral compound or non-salt compound). The desired salt of a basic compound may be prepared by methods known to those of skill in the art by treating the compound with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of basic compounds with amino acids, such as aspartate salts and glutamate salts, also can be prepared. The desired salt of an acidic compound can be prepared by methods known to those of skill in the art by treating the compound with a base. Examples of inorganic salts of acid compounds include, but are not limited to, alkali metal and alkaline earth salts, such as sodium salts, potassium salts, magnesium salts, and calcium salts; ammonium salts; and aluminum salts. Examples of organic salts of acid compounds include, but are not limited to, procaine, dibenzylamine, N-ethylpiperidine, N,N′-dibenzylethylenediamine, and triethylamine salts. Salts of acidic compounds with amino acids, such as lysine salts, also can be prepared. For lists of pharmaceutically acceptable salts, see, for example, P. H. Stahl and C. G. Wermuth (eds.) “Handbook of Pharmaceutical Salts, Properties, Selection and Use” Wiley-VCH, 2011 (ISBN: 978-3-90639-051-2). Several pharmaceutically acceptable salts are also disclosed in Berge, J. Pharm. Sci. 66:1 (1977).

Reference to a compound as described in a pharmaceutical composition, or to a compound as described in a claim to a pharmaceutical composition, refers to the compound described by the formula recited in the pharmaceutical composition, without the other elements of the pharmaceutical composition, that is, without carriers, excipients, etc.

The terms “pharmaceutical formulation” and “pharmaceutical composition,” as used herein, refer to preparations that are in such form as to permit the biological activity of the active ingredient to be effective, and that contain no additional components that are unacceptably toxic to an individual to which the formulation or composition would be administered. Such formulations or compositions may be sterile. Such formulations or compositions may be sterile, with the exception of the inclusion of an oncolytic virus.

“Excipients” as used herein include pharmaceutically acceptable excipients, carriers, vehicles, or stabilizers that are nontoxic to the cell or mammal being exposed thereto at the dosages and concentrations employed. Often the physiologically acceptable excipient is an aqueous pH buffered solution.

“Treating” or “treatment” of any disease or disorder as used herein refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “inhibits growth” (e.g. referring to cells, such as tumor cells) is intended to include any measurable decrease in cell growth (e.g., tumor cell growth) when contacted with a compound or combination described herein, as compared to the growth of the same cells not in contact with the same compound or combination. In certain embodiments, growth may be inhibited by at least about 10%, 20%, 30%, 40%, 50%, 60%, 70%, 80%, 90%, 99%, or 100%. The decrease in cell growth can occur by a variety of mechanisms, including but not limited to antibody internalization, apoptosis, necrosis, and/or effector function-mediated activity.

“Proliferation” is used herein to refer to the proliferation of a cell. Increased proliferation encompasses the production of a greater number of cells relative to a baseline value. Decreased proliferation encompasses the production of a reduced number of cells relative to a baseline value. In certain embodiments, the cell is an immune cell such as a T-cell and increased proliferation is desired. In certain embodiments, the cell is a cancer cell and reduced proliferation is desired.

As used herein, the term “subject” means a mammalian subject. Exemplary subjects include, but are not limited to, humans, monkeys, dogs, cats, mice, rats, cows, horses, camels, avians, goats, and sheep. In certain embodiments, the subject is a human. In certain embodiments, the subject has a disease that can be treated or diagnosed with an antibody provided herein. In certain embodiments, the disease is gastric carcinoma, colorectal carcinoma, renal cell carcinoma, cervical carcinoma, non-small cell lung carcinoma, ovarian cancer, breast cancer, triple-negative breast cancer, endometrial cancer, prostate cancer, and/or a cancer of epithelial origin.

An “effective amount” of an agent disclosed herein is an amount sufficient to carry out a specifically stated purpose. An “effective amount” may be determined empirically and in a routine manner, in relation to the stated purpose. An “effective amount” or an “amount sufficient” of an agent is that amount adequate to produce a desired biological effect, such as a beneficial result, including a beneficial clinical result. In certain embodiments, the term “effective amount” refers to an amount of an agent effective to “treat” a disease or disorder in an individual (e.g., a mammal such as a human).

### 2. Combinations

Provided herein are Cbl-b inhibitor compounds for use in combination with a second therapeutic agent that modulates cell division or cell differentiation activity. In certain embodiments, combination of a Cbl-b inhibitor compound with the second therapeutic agent yields substantially increased efficacy against solid tumors in vivo. In certain embodiments, combination of a Cbl-b inhibitor compound with the second therapeutic agent yields substantially increased efficacy against hematologic cancers in vivo. The Cbl-b inhibitor compound can be any Cbl-b inhibitor compound described herein. In particular embodiments, the second therapeutic agent is an immunostimulatory sequence. In particular embodiments, the second therapeutic agent is a CpG-C type oligonucleotide. In particular embodiments, the second therapeutic agent is a TLR7 agonist. In particular embodiments, the second therapeutic is a TLR9 agonist.

Generally, the Cbl-b inhibitor compound and the second therapeutic agent are administered according to their own doses and schedules. Thus, in certain embodiments, the Cbl-b inhibitor compound is administered at a dose and schedule deemed useful by the practitioner of skill. In certain embodiments, the second therapeutic agent is administered at a dose and schedule deemed useful by the practitioner of skill. In particular embodiments, the second therapeutic agent is administered according to its labelled instruction.

The combinations can be used for the treatment or prevention of any disease or disorder deemed suitable by the practitioner of skill. In certain embodiments, the Cbl-b inhibitor compound enhances a therapy provided by the second therapeutic agent. In certain embodiments, the second therapeutic agent enhances a therapy provided by Cbl-b inhibitor compound. In certain embodiments, the enhancement is synergistic. In certain embodiments, the disease or disorder is any disease or disorder suitable for treatment with the Cbl-b inhibitor compound. In certain embodiments, the disease or disorder is any disease or disorder suitable for treatment with the second therapeutic agent. In certain embodiments, the combination is for the treatment of a cancer. In certain embodiments, the combination is for the treatment of a solid tumor. In certain embodiments, the combination is for the treatment of a hematological cancer. Useful diseases and disorders are described herein.

In certain embodiments, the amount of the Cbl-b inhibitor compound is therapeutically effective. In certain embodiments, the amount of the second therapeutic agent is therapeutically effective. In certain embodiments, the amount of the Cbl-b inhibitor compound is therapeutically effective, and the amount of the second therapeutic agent is therapeutically effective. In certain embodiments, the amount of the Cbl-b inhibitor compound is sub-therapeutic. In certain embodiments, the amount of the second therapeutic agent is sub-therapeutic. In certain embodiments, the amount of the Cbl-b inhibitor compound is sub-therapeutic, and the amount of the second therapeutic agent is sub-therapeutic. In certain sub-therapeutic embodiments, the combination is therapeutic while one or more components are at sub-therapeutic doses.

In certain embodiments, the Cbl-b inhibitor compound and the additional therapeutic agent are administered consecutively in either order. As used herein, the terms “consecutively,” “serially,” and “sequentially” refer to administration of a Cbl-b inhibitor compound after an additional therapeutic agent, or administration of the additional therapeutic agent after the Cbl-b inhibitor compound. For instance, consecutive administration may involve administration of the Cbl-b inhibitor compound in the absence of the additional therapeutic agent during an induction phase (primary therapy), which is followed by a post-induction treatment phase comprising administration of the additional therapeutic agent. The methods may further comprise a maintenance phase comprising administration of the Cbl-b inhibitor compound or the additional therapeutic agent, or both. Alternatively, consecutive administration may involve administration of the additional therapeutic agent in the absence of the Cbl-b inhibitor compound during an induction phase (primary therapy), which is followed by a post-induction treatment phase comprising administration of the Cbl-b inhibitor compound. The methods may further comprise a maintenance phase comprising administration of the Cbl-b inhibitor compound or the additional therapeutic agent, or both.

In certain embodiments, the Cbl-b inhibitor compound and the additional therapeutic agent are administered concurrently. As used herein, the terms “concurrently,” “simultaneously,” and “in parallel” refer to administration of a Cbl-b inhibitor compound and an additional therapeutic agent during the same physician visit or during the same phase of treatment. For instance, both the Cbl-b inhibitor compound and the additional therapeutic agent may be administered during one or more of an induction phase, a treatment phase, and a maintenance phase. However, concurrent administration does not require that the Cbl-b inhibitor compound and the additional therapeutic agent be present together in a single formulation or pharmaceutical composition, or that the Cbl-b inhibitor compound and the additional therapeutic agent be administered at precisely the same time.

In certain embodiments, a combination provided herein can be administered directly to an individual to modulate an immune response, treat a disease or condition (e.g., cancer and/or abnormal cell proliferation) and/or inhibit Cbl-b activity and/or activate the TLR-mediated immune response in the individual.

In certain embodiments, provided herein is a method of modulating the immune response, the method comprising administering an effective amount of a combination provided herein to an individual to modulate the immune response in the individual. In certain embodiments, the individual has a cancer such as a hematologic cancer or non-hematological cancer described herein.

In certain embodiments, provided herein is a method of treating cancer responsive to inhibition of Cbl-b activity, the method comprising administering an effective amount of a combination provided herein to an individual to treat the cancer responsive to inhibition of Cbl-b activity. In certain embodiments, the cancer is a hematologic cancer or non-hematological cancer such as one described herein.

In certain embodiments, provided herein is a method of treating cancer responsive to agonism of TLR activity, the method comprising administering an effective amount of a combination provided herein to an individual to treat the cancer responsive to agonism of TLR activity. In certain embodiments, the cancer is a hematologic cancer or non-hematological cancer such as one described herein.

In certain embodiments, provided herein is a method of treating cancer that is nonresponsive to the agonism of TLR activity alone, the method comprising administering an effective amount of a combination provided herein to such an individual to treat the cancer nonresponsive to inhibition of agonism of TLR activity alone. In certain embodiments, the cancer is a hematologic cancer or non-hematological cancer such as one described herein.

In certain embodiments, provided herein is a method of inhibiting abnormal cell proliferation (e.g., hyperplasia), the method comprising administering an effective amount of a combination provided herein to an individual to inhibit abnormal cell proliferation in the individual.

In certain embodiments, provided herein is a method of inhibiting Cbl-b activity, the method comprising administering an effective amount of a combination provided herein to an individual to inhibit Cbl-b activity in the individual.

In certain embodiments, provided herein is a method of agonizing TLR activity, the method comprising administering an effective amount of a combination provided herein to an individual to agonize TLR in the individual.

In certain embodiments, provided herein is a method of inhibiting Cbl-b activity and TLR activity, the method comprising administering an effective amount of a combination provided herein to an individual to inhibit Cbl-b activity and TLR activity in the individual.

In certain embodiments, such as in the modulation of an immune response in an individual in need thereof (e.g., an individual with a T-cell dysfunction disorder), treatment of a disease or condition in an individual (e.g., an individual cancer and/or abnormal cell proliferation) and/or inhibition of Cbl-b or TLR activity in an individual, the appropriate dosage of an active agent will depend on the type of condition, disease, or disorder to be treated, as defined above, the severity and course of the condition, disease, or disorder, whether the agent is administered for preventive or therapeutic purposes, previous therapy, the subject's clinical history and response to the Cbl-b inhibitor compound or TLR agonist, and the discretion of the attending physician.

The Cbl-b inhibitor compound or composition thereof is suitably administered to the individual at one time or over a series of treatments. In certain embodiments, the treatment includes multiple administrations of the Cbl-b inhibitor compound or composition, wherein the interval between administrations may vary. For example, the interval between the first administration and the second administration is about one month, and the intervals between the subsequent administrations are about three months. In certain embodiments, a Cbl-b inhibitor compound is administered at a flat dose. In certain embodiments, a Cbl-b inhibitor compound described herein is administered to an individual at a fixed dose based on the individual's weight (e.g., mg/kg).

The TLR agonists or composition thereof is suitably administered to the individual at one time or over a series of treatments. In certain embodiments, the treatment includes multiple administrations of the TLR agonist or composition, wherein the interval between administrations may vary. For example, the interval between the first administration and the second administration is about one month, and the intervals between the subsequent administrations are about three months. In certain embodiments, a TLR agonists is administered at a flat dose. In certain embodiments, a TLR agonist is administered to an individual at a fixed dose based on the individual's weight (e.g., mg/kg).

In certain embodiments of this disclosure, the cancer is a hematologic cancer. For example, the hematologic cancer may be a lymphoma, a leukemia, or a myeloma. In other aspects of this disclosure, the cancer is a non-hematologic cancer. In particular, the non-hematologic cancer may be a carcinoma, a sarcoma, or a melanoma.

In certain embodiments, the effectiveness of the combination in the methods herein (e.g., method of modulating an immune response in an individual) can be assessed by measuring the biological activity of immune cells present in a sample (e.g., blood sample) isolated from the treated individual. For example, the ability of immune cells isolated from the individual after treatment with a combination provided herein to destroy target cells in a cytotoxicity assay may be measured to assess treatment efficacy. In certain embodiments, the biological activity of immune cells present in a sample (e.g., blood sample) can be measured by assaying expression and/or secretion of certain cytokines, such as IL-2 and IFNγ.

### 3. Cbl-b Compounds

In certain embodiments, the Cbl-b inhibitor compound is a compound of Formula (I)

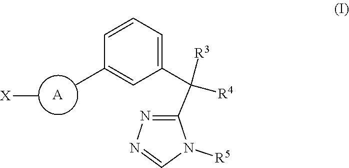

or a tautomer thereof, or a pharmaceutically acceptable salt thereof,
wherein

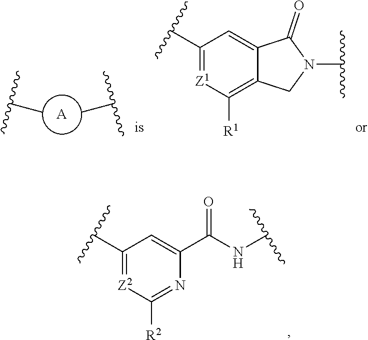

Z¹ is CH or N;
Z² is CH or N;
R¹ is —CF₃ or cyclopropyl;
R² is —CF₃ or cyclopropyl;
R³ is hydrogen, C₁-C₂ alkyl, or C₁-C₂ haloalkyl;
R⁴ is hydrogen, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by 1-5 R⁶ groups; or R³ and R⁴ are taken together with the carbon atom to which they are attached to form a spiro C₃-C₅ cycloalkyl or a spiro 4- to 6-membered heterocyclyl, each of which is optionally substituted by 1-5 R⁶ groups;
R⁵ is hydrogen, C₁-C₆ alkyl, C₁-C₆ haloalkyl, or C₃-C₆ cycloalkyl;
each R⁶ is independently C₁-C₆ alkyl, halo, hydroxy, —O—(C₁-C₆ alkyl), —CN, C₁-C₆ alkyl-CN, C₁-C₆ alkyl-OH, or C₁-C₆ haloalkyl;
or two R⁶ groups attached to the same carbon atom are taken together with the carbon atom to which they are attached to form a spiro C₃-C₆ cycloalkyl or a spiro 4- to 6-membered heterocyclyl;
X is hydrogen, C₁-C₆ alkyl, C₁-C₆ haloalkyl, C₁-C₆ alkyl-OH, C₁-C₆ alkyl-CN, C₃-C₆ cycloalkyl optionally substituted by 1-5 R⁸ groups, or

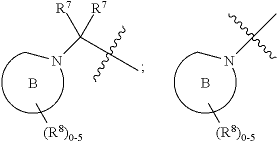

is a 4- to 7-membered heterocyclyl or a 5- to 8-membered heteroaryl, each of which heterocyclyl or heteroaryl optionally contains 1-2 additional heteroatoms selected from the group consisting of N, O, and S, and each of which heterocyclyl or heteroaryl is optionally substituted by 1-5 R⁸ groups;
each R⁷ is independently hydrogen, C₁-C₆ alkyl, C₁-C₆ alkyl-OH, or C₁-C₆ haloalkyl;
or two R⁷ groups are taken together with the carbon atom to which they are attached to form a spiro C₃-C₅ cycloalkyl or a spiro 3- to 5-membered heterocyclyl; and
each R⁸ is independently halo, C₁-C₆ alkyl, C₁-C₆ alkyl-CN, C₁-C₆ alkyl-OH, C₁-C₆ haloalkyl, —CN, oxo, or —O(C₁-C₆ alkyl);
or two R⁸ groups are taken together with the carbon atom or atoms to which they are attached to form a spiro or fused C₃-C₅ cycloalkyl or 3- to 5-membered heterocyclyl.

In certain embodiments,

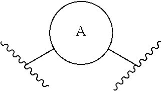

(i.e., the Ring A moiety), is

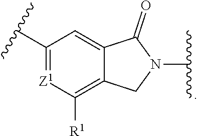

In certain embodiments, Z¹ is CH. In other embodiments, Z¹ is nitrogen. In certain embodiments, R¹ is —CF₃. In other embodiments, R¹ is cyclopropyl. In certain embodiments, the Ring A moiety is

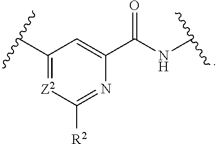

In certain embodiments, Z² is CH. In other embodiments, Z² is nitrogen. In certain embodiments, R² is —CF₃. In other embodiments, R² is cyclopropyl. In certain embodiments, the Ring A moiety is selected from the group consisting of:

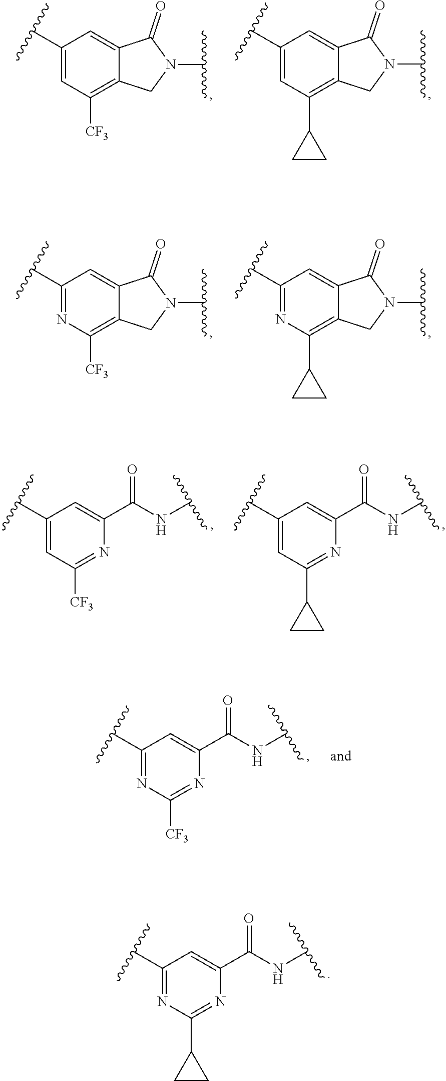

In certain embodiments, the Ring A moiety is

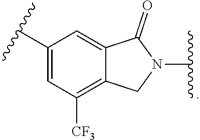 [SMILES: *c1cc2c(c(C(F)(F)F)c1)CN(*)C2=O]

In certain embodiments, the Ring A moiety

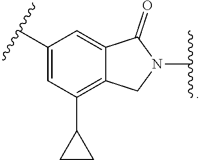 [SMILES: *c1cc2c(c(C3CC3)c1)CN(*)C2=O]

In certain embodiments, the Ring A moiety is

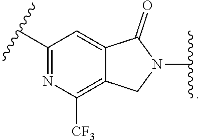 [SMILES: *c1cc2c(c(C(F)(F)F)n1)CN(*)C2=O]

In certain embodiments, the Ring A moiety is

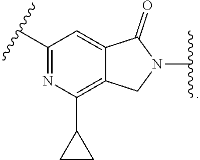 [SMILES: *c1cc2c(c(C3CC3)n1)CN(*)C2=O]

In certain embodiments, the Ring A moiety is

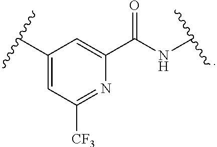 [SMILES: *NC(=O)c1cc(*)cc(C(F)(F)F)n1]

In certain embodiments, the Ring A moiety is

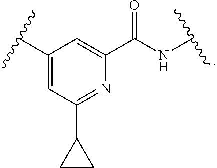 [SMILES: *NC(=O)c1cc(*)cc(C2CC2)n1]

In certain embodiments, the Ring A moiety is

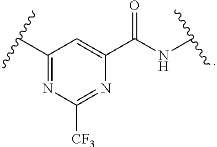 [SMILES: *NC(=O)c1cc(*)nc(C(F)(F)F)n1]

In certain embodiments, the Ring A moiety is

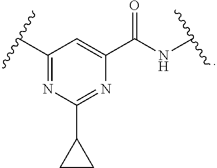 [SMILES: *NC(=O)c1cc(*)nc(C2CC2)n1]

In certain embodiments, R³ is hydrogen, C₁-C₂ alkyl, or C₁-C₂ haloalkyl. In certain embodiments, R³ is hydrogen, —CH₃, or —CF₃.

In certain embodiments, R³ is hydrogen.

In certain embodiments, R³ is C₁-C₂ alkyl. In certain embodiments, R³ is methyl. In certain embodiments, R³ is ethyl.

In certain embodiments, R³ is C₁-C₂ haloalkyl. In certain embodiments, R³ is C₁-C₂ haloalkyl containing 1-5 halogen atoms. In certain embodiments, R³ is C₁-C₂ haloalkyl containing 1-3 halogen atoms. In certain embodiments, R³ is C₁ haloalkyl. In certain embodiments, R³ is C₂ haloalkyl. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R³ is —CF₃, —CCl₃, —CF₂Cl, —CFCl₂, —CHF₂, —CH₂F, —CHCl₂, —CH₂Cl, or —CHFCl. In certain embodiments, R³ is —CF₃. In certain embodiments, R³ is —CCl₃. In certain embodiments, R³ is —CF₂Cl. In certain embodiments, R³ is —CFCl₂. In certain embodiments, R³ is —CHF₂. In certain embodiments, R³ is —CH₂F. In certain embodiments, R³ is —CHCl₂. In certain embodiments, R³ is —CH₂Cl. In certain embodiments, R³ is —CHFCl.

In certain embodiments, R⁴ is hydrogen, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by 1-5 R⁶ groups. In certain embodiments, R⁴ is hydrogen, C₁-C₃ alkyl, C₁-C₃ haloalkyl, 4- to 6-membered heterocyclyl, or C₄-C₅ cycloalkyl, wherein the heterocyclyl or cycloalkyl groups are optionally substituted by 1-3 R⁶ groups. In certain embodiments, R⁴ is hydrogen, —CH₃, —CF₃, cyclobutyl, or

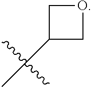 [SMILES: *C1COC1]

In certain embodiments, R⁴ is hydrogen. In certain embodiments, R⁴ is —CH₃. In certain embodiments, R⁴ is —CF₃. In certain embodiments, R⁴ is cyclobutyl. In certain embodiments, R⁴ is

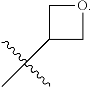 [SMILES: *C1COC1]

In certain embodiments, R⁴ is C₁-C₆ alkyl. In certain embodiments, R⁴ is C₁-C₃ alkyl. In certain embodiments, R⁴ is methyl, ethyl, n-propyl, or isopropyl. In certain embodiments, R⁴ is —CH₃. In certain embodiments, R⁴ is ethyl. In certain embodiments, R⁴ is n-propyl. In certain embodiments, R⁴ is isopropyl.

In certain embodiments, R⁴ is C₁-C₆ haloalkyl. In certain embodiments, R⁴ is C₁-C₆ haloalkyl containing 1-7 halogen atoms. In certain embodiments, R⁴ is C₁-C₃ haloalkyl. In certain embodiments, R⁴ is C₁-C₃ haloalkyl containing 1-7 halogen atoms. In certain embodiments, R⁴ is C₁-C₂ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁴ is —CF₃, —CCl₃, —CF₂Cl, —CFCl₂, —CHF₂, —CH₂F, —CHCl₂, —CH₂F, or —CHFCl. In certain embodiments, R⁴ is —CF₃. In certain embodiments, R⁴ is —CCl₃. In certain embodiments, R⁴ is —CF₂Cl. In certain embodiments, R⁴ is —CFCl₂. In certain embodiments, R⁴ is —CHF₂. In certain embodiments, R⁴ is —CH₂F. In certain embodiments, R⁴ is —CHCl₂. In certain embodiments, R⁴ is —CH₂F. In certain embodiments, R⁴ is —CHFCl.

In certain embodiments, R⁴ is 4- to 8-membered heterocyclyl optionally substituted by 1-5 R⁶ groups. In certain embodiments, R⁴ is 4- to 6-membered heterocyclyl optionally substituted by 1-3 R⁶ groups. In certain embodiments, R⁴ is a 4-membered heterocyclyl optionally substituted by 1-2 R⁶ groups. In certain embodiments, the heterocyclyl is substituted by five R⁶ groups. In certain embodiments, the heterocyclyl is substituted by four R⁶ groups. In certain embodiments, the heterocyclyl is substituted by three R⁶ groups. In certain embodiments, the heterocyclyl is substituted by two R⁶ groups. In certain embodiments, the heterocyclyl is substituted by one R⁶ group. In certain embodiments, the heterocyclyl is unsubstituted. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁴ is oxetanyl, azetidinyl, tetrahydrofuranyl, dioxolanyl, pyrrolidinyl, pyrazolidinyl, piperidinyl, isoxazolidinyl, or tetrahydropyranyl, each of which is optionally substituted by 1-5 R⁶ groups. In certain embodiments, R⁴ is

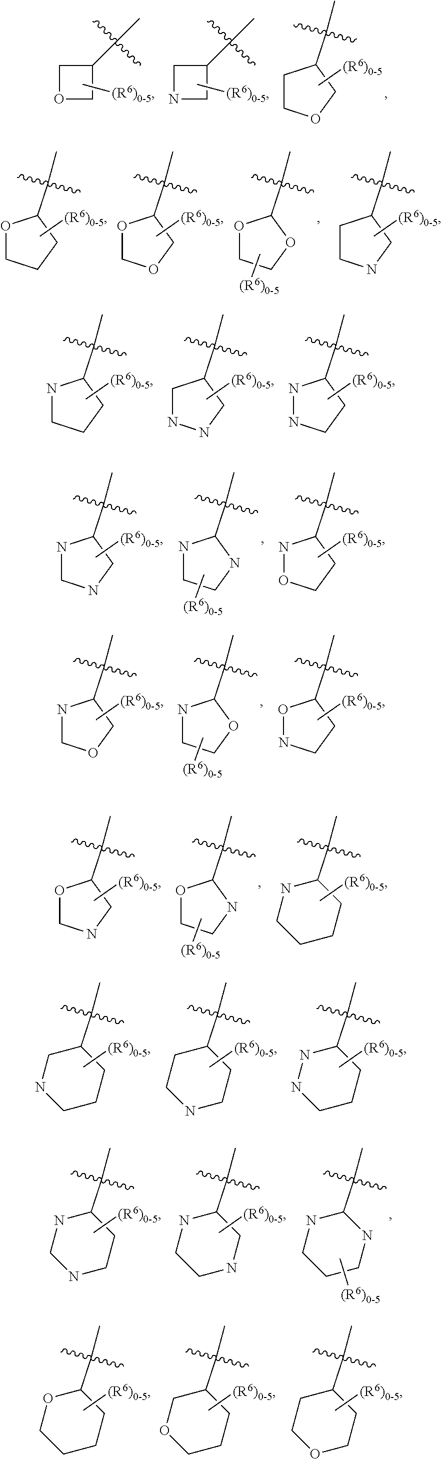

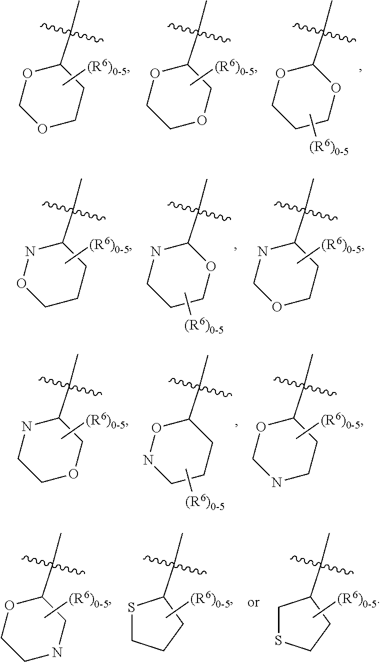

In certain embodiments, R⁴ is

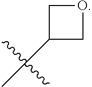 [SMILES: *C1COC1]

In certain embodiments, R⁴ is C₃-C₆ cycloalkyl optionally substituted by 1-5 R⁶ groups. In certain embodiments, R⁴ is C₄-C₅ cycloalkyl optionally substituted by 1-3 R⁶ groups. In certain embodiments, the cycloalkyl is substituted by five R⁶ groups. In certain embodiments, the cycloalkyl is substituted by four R⁶ groups. In certain embodiments, the cycloalkyl is substituted by three R⁶ groups. In certain embodiments, the cycloalkyl is substituted by two R⁶ groups. In certain embodiments, the cycloalkyl is substituted by one R⁶ group. In certain embodiments, the cycloalkyl is unsubstituted. In certain embodiments, R⁴ is cyclopropyl, cyclobutyl, cyclopentyl, or cyclohexyl, each of which is optionally substituted by 1-5 R⁶ groups. In certain embodiments, R⁴ is cyclopropyl or cyclobutyl. In certain embodiments, R⁴ is cyclobutyl. In certain embodiments, R⁴ is cyclopropyl.

In certain embodiments, R³ and R⁴ are taken together with the carbon atom to which they are attached to form a C₃-C₅ cycloalkyl or 4- to 6-membered heterocyclyl, each of which is optionally substituted by 1-5 R⁶ groups. In certain embodiments, R³ and R⁴ are taken together with the carbon atom to which they are attached to form a C₄-C₅ cycloalkyl or 4- to 6-membered heterocyclyl, each of which is optionally substituted by 1-3 R⁶ groups. In certain embodiments, R³ and R⁴ are taken together with the carbon atom to which they are attached to form

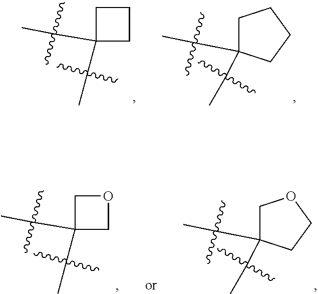 [SMILES: *C1(*)CCC1 ; *C1(*)CCCC1 ; *C1(*)COC1 ; *C1(*)CCOC1]

each of which is optionally substituted by 1-3 R⁶ groups. In certain embodiments, R³ and R⁴ are taken together with the carbon atom to which they are attached, and are substituted by one R⁶ group which is methyl, to form

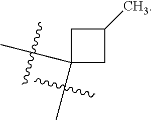 [SMILES: *C1(*)CC(C)C1]

In certain embodiments, R³ and R⁴ are taken together with the carbon atom to which they are attached to form a C₃-C₅ cycloalkyl optionally substituted by 1-5 R⁶ groups. In certain embodiments, R³ and R⁴ are taken together with the carbon atom to which they are attached to form a C₄-C₅ cycloalkyl optionally substituted by 1-3 R⁶ groups. In certain embodiments, the cycloalkyl is substituted by five R⁶ groups. In certain embodiments, the cycloalkyl is substituted by four R⁶ groups. In certain embodiments, the cycloalkyl is substituted by three R⁶ groups. In certain embodiments, the cycloalkyl is substituted by two R⁶ groups. In certain embodiments, the cycloalkyl is substituted by one R⁶ group. In certain embodiments, the cycloalkyl is unsubstituted. In certain embodiments, R³ and R⁴ are taken together with the carbon atom to which they are attached to form

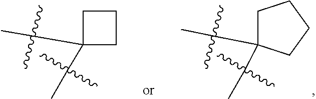 [SMILES: *C1(*)CCC1 ; *C1(*)CCCC1]

each of which is optionally substituted by 1-3 R⁶ groups. In certain embodiments, R³ and R⁴ are taken together with the carbon atom to which they are attached, and are substituted by one R⁶ group which is methyl, to form

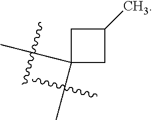 [SMILES: *C1(*)CC(C)C1]

In certain embodiments, the absolute stereochemistry at the carbon atom to which the methyl group of

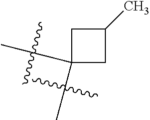 [SMILES: *C1(*)CC(C)C1]

is attached is (R)— (i.e., using the Cahn-Ingold-Prelog rules). In certain embodiments, the absolute stereochemistry at the carbon atom to which the methyl group of

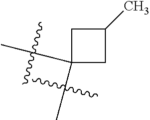 [SMILES: *C1(*)CC(C)C1]

is attached is (S)—.

In certain embodiments, R³ and R⁴ are taken together with the carbon atom to which they are attached to form a 4- to 6-membered heterocyclyl optionally substituted by 1-5 R⁶ groups. In certain embodiments, R³ and R⁴ are taken together with the carbon atom to which they are attached to form a 4- to 6-membered heterocyclyl optionally substituted by 1-3 R⁶ groups. In certain embodiments, the heterocyclyl is substituted by five R⁶ groups. In certain embodiments, the heterocyclyl is substituted by four R⁶ groups. In certain embodiments, the heterocyclyl is substituted by three R⁶ groups. In certain embodiments, the heterocyclyl is substituted by two R⁶ groups. In certain embodiments, the heterocyclyl is substituted by one R⁶ group. In certain embodiments, the heterocyclyl is unsubstituted. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁴ is oxetanyl, azetidinyl, tetrahydrofuranyl, dioxolanyl, pyrrolidinyl, pyrazolidinyl, piperidinyl, isoxazolidinyl, or tetrahydropyranyl, each of which is optionally substituted by 1-5 R⁶ groups. In certain embodiments, R³ and R⁴ are taken together with the carbon atom to which they are attached to form

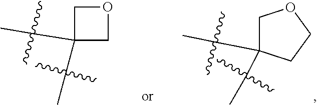 [SMILES: *C1(*)COC1 ; *C1(*)CCOC1]

each of which is optionally substituted by 1-3 R⁶ groups. In certain embodiments, R³ and R⁴ are taken together with the carbon atom to which they are attached to form

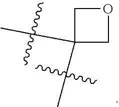 [SMILES: *C1(*)COC1]

In certain embodiments, R³ and R⁴ are taken together with the carbon atom to which they are attached to form

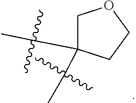 [SMILES: *C1(*)CCOC1]

In certain embodiments, each R⁶ is independently C₁-C₆ alkyl, halo, hydroxy, —O—(C₁-C₆ alkyl), —CN, C₁-C₆ alkyl-CN, C₁-C₆ alkyl-OH, or C₁-C₆ haloalkyl. In certain embodiments, each R⁶ is independently C₁-C₃ alkyl, halo, hydroxy, —O—(C₁-C₃ alkyl), —CN, C₁-C₃ alkyl-CN, C₁-C₃ alkyl-OH, or C₁-C₃ haloalkyl. In certain embodiments, each R⁶ is independently —CH₃, fluoro (F), hydroxy, —OCH₃, —CN, —CH₂CN, —CH₂OH, or —CF₃.

In certain embodiments, R⁶ is C₁-C₆ alkyl. In certain embodiments, R⁶ is C₁-C₃ alkyl. In certain embodiments, R⁶ is methyl, ethyl, n-propyl, or isopropyl. In certain embodiments, R⁶ is —CH₃. In certain embodiments, R⁶ is ethyl. In certain embodiments, R⁶ is n-propyl. In certain embodiments, R⁶ is isopropyl.

In certain embodiments, R⁶ is halo. In certain embodiments, R⁶ is chloro, fluoro, or bromo. In certain embodiments, R⁶ is chloro or fluoro. In certain embodiments, R⁷ is fluoro. In certain embodiments, R⁶ is chloro. In certain embodiments, R⁶ is bromo.

In certain embodiments, R⁶ is hydroxyl.

In certain embodiments, R⁶ is —O(C₁-C₆ alkyl). In certain embodiments, R⁶ is —O—(C₁-C₃ alkyl). In certain embodiments, R⁶ is —O(methyl), —O(ethyl), —O(n-propyl), or —O(isopropyl). In certain embodiments, R⁶ is —OCH₃ or —OCH₂CH₃. In certain embodiments, R⁶ is —OCH₃. In certain embodiments, R⁶ is —OCH₂CH₃. In certain embodiments, R⁶ is —OCH₂CH₂CH₃. In certain embodiments, R⁶ is —OCH(CH₃)₂.

In certain embodiments, R⁶ is —CN. In certain embodiments, R⁶ is C₁-C₆ alkyl-CN. In certain embodiments, R⁶ is C₁-C₃ alkyl-CN. In certain embodiments, R⁶ is —CH₂CN, —CH₂CH₂—CN, —CH₂CH₂CH₂—CN, or —C(CH₃)₂—CN. In certain embodiments, R⁶ is —CH₂CN. In certain embodiments, R⁶ is —CH₂CH₂—CN. In certain embodiments, R⁶ is —CH₂CH₂CH₂—CN. In certain embodiments, R⁶ is —C(CH₃)₂—CN.

In certain embodiments, R⁶ is C₁-C₆ alkyl-OH. In certain embodiments, R⁶ is C₁-C₃ alkyl-OH. In certain embodiments, R⁶ is —CH₂OH, —CH₂CH₂—OH, —CH₂CH₂CH₂—OH, or —C(CH₃)₂—OH. In certain embodiments, R⁶ is —CH₂OH. In certain embodiments, R⁶ is —CH₂CH₂—OH. In certain embodiments, R⁶ is —CH₂CH₂CH₂—OH. In certain embodiments, R⁶ is —C(CH₃)₂—OH.

In certain embodiments, R⁶ is C₁-C₆ haloalkyl. In certain embodiments, R⁶ is C₁-C₆ haloalkyl containing 1-7 halogen atoms. In certain embodiments, R⁶ is C₁-C₃ haloalkyl. In certain embodiments, R⁶ is C₁-C₃ haloalkyl containing 1-7 halogen atoms. In certain embodiments, R⁶ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁶ is —CF₃, —CCl₃, —CF₂Cl, —CFCl₂, —CHF₂, —CH₂F, —CHCl₂, —CH₂Cl, or —CHFCl. In certain embodiments, R⁶ is —CF₃. In certain embodiments, R⁶ is —CCl₃. In certain embodiments, R⁶ is —CF₂Cl. In certain embodiments, R⁶ is —CFCl₂. In certain embodiments, R⁶ is —CHF₂. In certain embodiments, R⁶ is —CH₂F. In certain embodiments, R⁶ is —CHCl₂. In certain embodiments, R⁶ is —CH₂Cl. In certain embodiments, R⁶ is —CHFCl.

In certain embodiments, two R⁶ groups attached to the same carbon atom are taken together with the carbon atom to which they are attached to form a spiro C₃-C₆ cycloalkyl or spiro 4- to 6-membered heterocyclyl. In certain embodiments, two R⁶ groups attached to the same carbon atom are taken together with the carbon atom to which they are attached to form a spiro C₃-C₆ cycloalkyl or spiro 4- to 5-membered heterocyclyl.

In certain embodiments, two R⁶ groups attached to the same carbon atom are taken together with the carbon atom to which they are attached to form a spiro C₃-C₆ cycloalkyl. In certain embodiments, two R⁶ groups attached to the same carbon atom are taken together with the carbon atom to which they are attached to form a spiro C₃-C₅ cycloalkyl. In certain embodiments, two R⁶ groups attached to the same carbon atom are taken together with the carbon atom to which they are attached to form a spiro C₃-C₄ cycloalkyl. In certain embodiments, two R⁶ groups attached to the same carbon atom are taken together with the carbon atom to which they are attached to form a spiro cyclopropyl, cyclobutyl, or cyclopentyl. In certain embodiments, two R⁶ groups attached to the same carbon atom are taken together with the carbon atom to which they are attached to form a spiro cyclopropyl. In certain embodiments, two R⁶ groups attached to the same carbon atom are taken together with the carbon atom to which they are attached to form a spiro cyclobutyl. In certain embodiments, two R⁶ groups attached to the same carbon atom are taken together with the carbon atom to which they are attached to form a spiro cyclopentyl.

In certain embodiments, two R⁶ groups attached to the same carbon atom are taken together with the carbon atom to which they are attached to form a spiro 4- to 6-membered heterocyclyl. In certain embodiments, two R⁶ groups attached to the same carbon atom are taken together with the carbon atom to which they are attached to form a spiro 4- to 5-membered heterocyclyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, two R⁶ groups attached to the same carbon atom are taken together with the carbon atom to which they are attached to form a spiro oxetanyl, azetidinyl, tetrahydrofuranyl, dioxolanyl, pyrrolidinyl, pyrazolidinyl, piperidinyl, isoxazolidinyl, or tetrahydropyranyl. In certain embodiments, two R⁶ groups attached to the same carbon atom are taken together with the carbon atom to which they are attached to form a spiro oxetanyl. In certain embodiments, two R⁶ groups attached to the same carbon atom are taken together with the carbon atom to which they are attached to form a spiro azetidinyl. In certain embodiments, two R⁶ groups attached to the same carbon atom are taken together with the carbon atom to which they are attached to form a spiro tetrahydrofuranyl. In certain embodiments, two R⁶ groups attached to the same carbon atom are taken together with the carbon atom to which they are attached to form a spiro dioxolanyl. In certain embodiments, two R⁶ groups attached to the same carbon atom are taken together with the carbon atom to which they are attached to form a spiro pyrrolidinyl. In certain embodiments, two R⁶ groups attached to the same carbon atom are taken together with the carbon atom to which they are attached to form a spiro pyrazolidinyl. In certain embodiments, two R⁶ groups attached to the same carbon atom are taken together with the carbon atom to which they are attached to form a spiro piperidinyl. In certain embodiments, two R⁶ groups attached to the same carbon atom are taken together with the carbon atom to which they are attached to form a spiro isoxazolidinyl. In certain embodiments, two R⁶ groups attached to the same carbon atom are taken together with the carbon atom to which they are attached to form a spiro tetrahydropyranyl.

In certain embodiments, R⁵ is hydrogen, C₁-C₆ alkyl, C₁-C₆ haloalkyl, or C₃-C₆ cycloalkyl. In certain embodiments, R⁵ is hydrogen, C₁-C₃ alkyl, C₁-C₃ haloalkyl, or C₃-C₄ cycloalkyl. In certain embodiments, R⁵ is hydrogen, —CH₃, —CHF₂, or cyclopropyl.

In certain embodiments, R⁵ is hydrogen. In certain embodiments, R⁵ is —CH₃. In certain embodiments, R⁵ is —CHF₂. In certain embodiments, R⁵ is cyclopropyl.

In certain embodiments, R⁵ is C₁-C₆ alkyl. In certain embodiments, R⁵ is C₁-C₃ alkyl. In certain embodiments, R⁵ is methyl, ethyl, n-propyl, or isopropyl. In certain embodiments, R⁵ is —CH₃. In certain embodiments, R⁵ is ethyl. In certain embodiments, R⁵ is n-propyl. In certain embodiments, R⁵ is isopropyl.

In certain embodiments, R⁵ is C₁-C₆ haloalkyl. In certain embodiments, R⁵ is C₁-C₆ haloalkyl containing 1-7 halogen atoms. In certain embodiments, R⁵ is C₁-C₃ haloalkyl. In certain embodiments, R⁵ is C₁-C₃ haloalkyl containing 1-7 halogen atoms. In certain embodiments, R⁵ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁵ is —CF₃, —CCl₃, —CF₂Cl, —CFCl₂, —CHF₂, —CH₂F, —CHCl₂, —CH₂Cl, or —CHFCl. In certain embodiments, R⁵ is —CHF₂. In certain embodiments, R⁵ is —CF₃. In certain embodiments, R⁵ is —CCl₃. In certain embodiments, R⁵ is —CF₂Cl. In certain embodiments, R⁵ is —CFCl₂. In certain embodiments, R⁵ is —CH₂F. In certain embodiments, R⁵ is —CHCl₂. In certain embodiments, R⁵ is —CH₂Cl. In certain embodiments, R⁵ is —CHFCl.

In certain embodiments, R⁵ is C₃-C₆ cycloalkyl. In certain embodiments, R⁵ is C₃-C₅ cycloalkyl. In certain embodiments, R⁵ is C₃-C₄ cycloalkyl. In certain embodiments, R⁵ is cyclopropyl, cyclobutyl, or cyclopentyl. In certain embodiments, R⁵ is cyclopropyl. In certain embodiments, R⁵ is cyclobutyl. In certain embodiments, R⁵ is cyclopentyl.

In certain embodiments, X is hydrogen, C₁-C₆ alkyl, C₁-C₆ haloalkyl, C₁-C₆ alkyl-OH, C₁-C₆ alkyl-CN, or C₃-C₆ cycloalkyl optionally substituted by 1-5 R⁸ groups. In certain embodiments, X is hydrogen, C₁-C₃ alkyl, C₁-C₃ haloalkyl, C₁-C₃ alkyl-OH, C₁-C₃ alkyl-CN, or C₃-C₅ cycloalkyl optionally substituted by 1-3 R⁸ groups. In certain embodiments, X is hydrogen or —CH₃.

In certain embodiments, X is hydrogen. In certain embodiments, X is methyl.

In certain embodiments, X is C₁-C₆ alkyl. In certain embodiments, X is C₁-C₃ alkyl. In certain embodiments, X is methyl, ethyl, n-propyl, or isopropyl. In certain embodiments, X is ethyl. In certain embodiments, X is n-propyl. In certain embodiments, X is isopropyl.

In certain embodiments, X is C₁-C₆ haloalkyl. In certain embodiments, X is C₁-C₆ haloalkyl containing 1-7 halogen atoms. In certain embodiments, X is C₁-C₃ haloalkyl. In certain embodiments, X is C₁-C₃ haloalkyl containing 1-7 halogen atoms. In certain embodiments, X is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, X is —CF₃, —CCl₃, —CF₂Cl, —CFCl₂, —CHF₂, —CH₂F, —CHCl₂, —CH₂Cl, or —CHFCl. In certain embodiments, X is —CF₃. In certain embodiments, X is —CCl₃. In certain embodiments, X is —CF₂Cl. In certain embodiments, X is —CFCl₂. In certain embodiments, X is —CHF₂. In certain embodiments, X is —CH₂F. In certain embodiments, X is —CHCl₂. In certain embodiments, X is —CH₂Cl. In certain embodiments, X is —CHFCl.

In certain embodiments, X is C₁-C₆ alkyl-OH. In certain embodiments, X is C₁-C₃ alkyl-OH. In certain embodiments, X is —CH₂OH, —CH₂CH₂—OH, —CH₂CH₂CH₂—OH, or —C(CH₃)₂—OH. In certain embodiments, X is —CH₂OH.

In certain embodiments, X is C₁-C₆ alkyl-CN. In certain embodiments, X is C₁-C₃ alkyl-CN. In certain embodiments, X is —CH₂CN, —CH₂CH₂—CN, —CH₂CH₂CH₂—CN, or —C(CH₃)₂—CN. In certain embodiments, X is —CH₂CN. In certain embodiments, X is —CH₂CH₂—CN. In certain embodiments, X is —CH₂CH₂CH₂—CN. In certain embodiments, X is —C(CH₃)₂—CN.

In certain embodiments, X is C₃-C₆ cycloalkyl optionally substituted by 1-5 R⁸ groups. In certain embodiments, X is C₃-C₅ cycloalkyl optionally substituted by 1-3 R⁸ groups. In certain embodiments, the cycloalkyl is substituted by five R⁸ groups. In certain embodiments, the cycloalkyl is substituted by four R⁸ groups. In certain embodiments, the cycloalkyl is substituted by three R⁸ groups. In certain embodiments, the cycloalkyl is substituted by two R⁸ groups. In certain embodiments, the cycloalkyl is substituted by one R⁸ group. In certain embodiments, the cycloalkyl is unsubstituted. In certain embodiments, X is cyclopropyl, cyclobutyl, or cyclopentyl, each of which is optionally substituted by 1-5 R⁸ groups. In certain embodiments, X is cyclopropyl. In certain embodiments, X is cyclobutyl, In certain embodiments, X is cyclopentyl,

In certain embodiments, X is

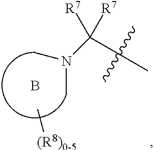

wherein the Ring B moiety, shown as

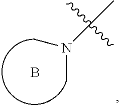

is a 4- to 7-membered heterocyclyl or a 5- to 8-membered heteroaryl, each of which heterocyclyl or heteroaryl optionally contains 1-2 additional heteroatoms selected from the group consisting of nitrogen, oxygen, or sulfur, and each of which heterocyclyl or heteroaryl is optionally substituted by 1-5 R⁸ groups. In certain embodiments, the Ring B moiety is a 4- to 6-membered heterocyclyl or a 5- to 6-membered heteroaryl, each of which heterocyclyl or heteroaryl optionally contains 1-2 additional heteroatoms selected from the group consisting of nitrogen, oxygen, or sulfur, and each of which heterocyclyl or heteroaryl is optionally substituted by 1-5 R⁸ groups. In certain embodiments, the Ring B moiety is a 4- to 5-membered heterocyclyl or a 5- to 6-membered heteroaryl, each of which heterocyclyl or heteroaryl optionally contains one additional heteroatom selected from the group consisting of nitrogen and oxygen, and each of which heterocyclyl or heteroaryl is optionally substituted by 1-5 R⁸ groups.

In certain embodiments, the Ring B moiety is a 4- to 7-membered heterocyclyl optionally containing 1-2 additional heteroatoms selected from the group consisting of nitrogen, oxygen, and sulfur, wherein the heterocyclyl is optionally substituted by 1-5 R⁸ groups. In certain embodiments, the Ring B moiety is a 4- to 6-membered heterocyclyl optionally containing 1-2 additional heteroatoms selected from the group consisting of nitrogen, oxygen, and sulfur, wherein the heterocyclyl is optionally substituted by 1-5 R⁸ groups. In certain embodiments, the Ring B moiety is a 4- to 5-membered heterocyclyl optionally containing one additional heteroatom selected from the group consisting of nitrogen and oxygen, wherein the heterocyclyl is optionally substituted by 1-5 R⁸ groups. In certain embodiments, the heterocyclyl is substituted by five R⁸ groups. In certain embodiments, the heterocyclyl is substituted by four R⁸ groups. In certain embodiments, the heterocyclyl is substituted by three R⁸ groups. In certain embodiments, the heterocyclyl is substituted by two R⁸ groups. In certain embodiments, the heterocyclyl is substituted by one R group. In certain embodiments, the heterocyclyl is unsubstituted. In certain embodiments, the heterocyclyl contains 1-2 additional heteroatoms selected from the group consisting of nitrogen, oxygen, and sulfur. In certain embodiments, the heterocyclyl contains one additional nitrogen atom. In certain embodiments, the heterocyclyl contains two additional nitrogen atoms. In certain embodiments, the heterocyclyl further contains one oxygen atom. In certain embodiments, the heterocyclyl further contains two oxygen atoms. In certain embodiments, the heterocyclyl further contains one oxygen atom and one nitrogen atom. In certain embodiments, the heterocyclyl further contains one sulfur atom. In certain embodiments, the heterocyclyl does not contain additional heteroatoms. In certain embodiments, the heterocyclyl is azetidinyl, pyrrolidinyl, pyrazolidinyl, piperidinyl, or isoxazolidinyl, each of which is optionally substituted by 1-5 R⁸ groups.

In certain embodiments, the Ring B moiety is a 5- to 8-membered heteroaryl optionally containing 1-2 additional heteroatoms selected from the group consisting of nitrogen, oxygen, and sulfur, wherein the heteroaryl is optionally substituted by 1-5 R⁸ groups. In certain embodiments, the Ring B moiety is a 5- to 6-membered heteroaryl optionally containing 1-2 additional heteroatoms selected from the group consisting of nitrogen, oxygen, and sulfur, wherein the heteroaryl is optionally substituted by 1-5 R⁸ groups. In certain embodiments, the Ring B moiety is a 5- to 6-membered heteroaryl optionally containing one additional heteroatom selected from the group consisting of nitrogen and oxygen, wherein the heteroaryl is optionally substituted by 1-5 R⁸ groups. In certain embodiments, the heteroaryl is substituted by five R⁸ groups. In certain embodiments, the heteroaryl is substituted by four R⁸ groups. In certain embodiments, the heteroaryl is substituted by three R⁸ groups. In certain embodiments, the heteroaryl is substituted by two R⁸ groups. In certain embodiments, the heteroaryl is substituted by one R⁸ group. In certain embodiments, the heteroaryl is unsubstituted. In certain embodiments, the heteroaryl contains 1-2 additional heteroatoms selected from the group consisting of nitrogen, oxygen, and sulfur. In certain embodiments, the heteroaryl contains one additional nitrogen atom. In certain embodiments, the heteroaryl contains two additional nitrogen atoms. In certain embodiments, the heteroaryl further contains one oxygen atom. In certain embodiments, the heteroaryl further contains two oxygen atoms. In certain embodiments, the heteroaryl further contains one oxygen atom and one additional nitrogen atom. In certain embodiments, the heteroaryl further contains one sulfur atom. In certain embodiments, the heteroaryl does not contain additional heteroatoms. In certain embodiments, the heteroaryl is pyrrolyl, imidazolyl, pyrazolyl, isoxazolyl, oxazolyl, thiazolyl, isothiazolyl, triazolyl, pyridinyl, pyridazinyl, pyrimidinyl, pyrazinyl, or triazyl, each of which is optionally substituted by 1-5 R⁸ groups.

In certain embodiments, the Ring B moiety is

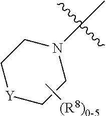

wherein Y is oxygen, —CH₂—, CHR⁸, or C(R⁸)₂, and X is

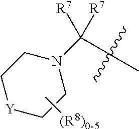

overall. In certain embodiments, Y is oxygen (O). In other embodiments, Y is —CH₂—, CHR⁸, or C(R⁸)₂. In certain embodiments, Y is —CH₂—. In certain embodiments, Y is CHR⁸. In certain embodiments, Y is C(R⁸)₂. In certain embodiments, the Ring B moiety is substituted by a total of 1-5 R⁸ groups. In certain embodiments, the Ring B moiety is substituted by a total of 1-3 R⁸ groups. As such, if Y is CHR⁸, then the Ring B moiety can be substituted by up to four additional R⁸ groups. Similarly, if Y is CH(R⁸)₂, then the Ring B moiety can be substituted by up to three additional R⁸ groups. In certain embodiments, the Ring B moiety is substituted by five R⁸ groups. In certain embodiments, the Ring B moiety is substituted by four R⁸ groups. In certain embodiments, the Ring B moiety is substituted by three R⁸ groups. In certain embodiments, the Ring B moiety is substituted by two R⁸ groups. In certain embodiments, the Ring B moiety is substituted by one R⁸ group. In certain embodiments, the Ring B moiety is unsubstituted. In certain embodiments, the Ring B moiety is

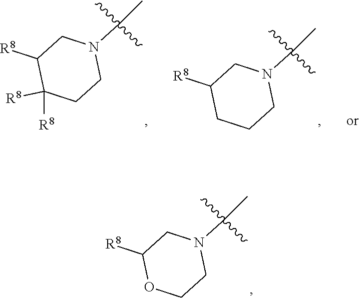

wherein each R⁸ is independently as described herein.

In certain embodiments, each R⁷ is independently hydrogen, C₁-C₆ alkyl, C₁-C₆ alkyl-OH, or C₁-C₆ haloalkyl. In certain embodiments, each R⁷ is independently hydrogen, C₁-C₃ alkyl, C₁-C₃ alkyl-OH, or C₁-C₃ haloalkyl. In certain embodiments, each R⁷ is independently hydrogen, —CH₃, —CH₂OH, or —CF₃.

In certain embodiments, both R⁷ groups are hydrogen (H). In certain embodiments, one R⁷ group is hydrogen. In certain embodiments, one R⁷ group is hydrogen, and the other R⁷ group is C₁-C₆ alkyl, C₁-C₆ alkyl-OH, or C₁-C₆ haloalkyl. In certain embodiments, one R⁷ group is hydrogen and the other R⁷ group is C₁-C₆ alkyl. In certain embodiments, one R⁷ group is hydrogen and the other R⁷ group is C₁-C₃ alkyl. In certain embodiments, one R⁷ group is hydrogen and the other R⁷ group is —CH₃.

In certain embodiments, R⁷ is C₁-C₆ alkyl. In certain embodiments, R⁷ is C₁-C₃ alkyl. In certain embodiments, R⁷ is methyl, ethyl, n-propyl, or isopropyl. In certain embodiments, one R⁷ group is methyl, ethyl, n-propyl, or isopropyl, and the other R⁷ group is hydrogen. In certain embodiments, R⁷ is —CH₃.

In certain embodiments, R⁷ is C₁-C₆ alkyl-OH. In certain embodiments, R⁷ is C₁-C₃ alkyl-OH. In certain embodiments, R⁷ is —CH₂OH, —CH₂CH₂—OH, —CH₂CH₂CH₂—OH, or —C(CH₃)₂—OH. In certain embodiments, R⁷ is —CH₂OH. In certain embodiments, R⁷ is —CH₂CH₂—OH. In certain embodiments, R⁷ is —CH₂CH₂CH₂—OH. In certain embodiments, R⁷ is —C(CH₃)₂—OH. In certain embodiments, one R⁷ group is C₁-C₆ alkyl-OH, and the other R⁷ group is hydrogen. In certain embodiments, one R⁷ group is C₁-C₃ alkyl-OH, and the other R⁷ group is hydrogen. In certain embodiments, one R⁷ group is —CH₂OH, —CH₂CH₂—OH, —CH₂CH₂CH₂—OH, or —C(CH₃)₂—OH, and the other R⁷ group is hydrogen. In certain embodiments, one R⁷ group is —CH₂OH, and the other R⁷ group is hydrogen. In certain embodiments, one R⁷ group is —CH₂CH₂—OH, and the other R⁷ group is hydrogen. In certain embodiments, one R⁷ group is —CH₂CH₂CH₂—OH, and the other R⁷ group is hydrogen. In certain embodiments, one R⁷ group is —C(CH₃)₂—OH, and the other R⁷ group is hydrogen.

In certain embodiments, R⁷ is C₁-C₆ haloalkyl. In certain embodiments, R⁷ is C₁-C₆ haloalkyl containing 1-7 halogen atoms. In certain embodiments, R⁷ is C₁-C₃ haloalkyl. In certain embodiments, R⁷ is C₁-C₃ haloalkyl containing 1-7 halogen atoms. In certain embodiments, R⁷ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁷ is —CF₃, —CCl₃, —CF₂Cl, —CFCl₂, —CHF₂, —CH₂F, —CHCl₂, —CH₂F, or —CHFCl. In certain embodiments, R⁷ is —CF₃. In certain embodiments, R⁷ is —CCl₃. In certain embodiments, R⁷ is —CF₂Cl. In certain embodiments, R⁷ is —CFCl₂. In certain embodiments, R⁷ is —CHF₂. In certain embodiments, R⁷ is —CH₂F. In certain embodiments, R⁷ is —CHCl₂. In certain embodiments, R⁷ is —CH₂F. In certain embodiments, R⁷ is —CHFCl.

In certain embodiments, two R⁷ groups are taken together with the carbon atom to which they are attached to form a C₃-C₅ cycloalkyl or a 3- to 5-membered heterocyclyl. In certain embodiments, two R⁷ groups are taken together with the carbon atom to which they are attached to form cyclopropyl or oxetanyl. In certain embodiments, two R⁷ groups are taken together with the carbon atom to which they are attached to form cyclopropyl. In certain embodiments, two R⁷ groups are taken together with the carbon atom to which they are attached to form oxetanyl.

In certain embodiments, two R⁷ groups are taken together with the carbon atom to which they are attached to form C₃-C₅ cycloalkyl. In certain embodiments, two R⁷ groups are taken together with the carbon atom to which they are attached to form cyclopropyl or cyclobutyl. In certain embodiments, two R⁷ groups are taken together with the carbon atom to which they are attached to form cyclopropyl. In certain embodiments, two R⁷ groups are taken together with the carbon atom to which they are attached to form cyclobutyl.

In certain embodiments, two R⁷ groups are taken together with the carbon atom to which they are attached to form a 3- to 5-membered heterocyclyl. In certain embodiments, two R⁷ groups are taken together with the carbon atom to which they are attached to form a 3- to 4-membered heterocyclyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, R⁷ is aziridinyl, oxiranyl, oxetanyl, azetidinyl, tetrahydrofuranyl, dioxolanyl, pyrrolidinyl, pyrazolidinyl, or isoxazolidinyl. In certain embodiments, R⁷ is aziridinyl. In certain embodiments, R⁷ is oxiranyl. In certain embodiments, R⁷ is oxetanyl. In certain embodiments, R⁷ is azetidinyl. In certain embodiments, R⁷ is tetrahydrofuranyl. In certain embodiments, R⁷ is dioxolanyl. In certain embodiments, R⁷ is pyrrolidinyl. In certain embodiments, R⁷ is pyrazolidinyl. In certain embodiments, R⁷ is isoxazolidinyl.

In certain embodiments, each R⁸ is independently halo, C₁-C₆ alkyl, C₁-C₆ alkyl-CN, C₁-C₆ alkyl-OH, C₁-C₆ haloalkyl, —CN, oxo, or —O(C₁-C₆ alkyl). In certain embodiments, each R⁸ is independently halo, C₁-C₃ alkyl, C₁-C₃ alkyl-CN, C₁-C₃ alkyl-OH, C₁-C₃ haloalkyl, —CN, oxo, or —O(C₁-C₃ alkyl). In certain embodiments, each R⁸ is independently fluoro (F), —CH₃, —CH₂CH₃, —CH₂CN, —CH₂OH, —CF₃, —CN, oxo, or —OCH₃.

In certain embodiments, R⁸ is halo. In certain embodiments, R⁸ is chloro, fluoro, or bromo. In certain embodiments, R⁸ is chloro or fluoro. In certain embodiments, R⁸ is fluoro. In certain embodiments, R⁸ is chloro. In certain embodiments, R⁸ is bromo.

In certain embodiments, R⁸ is C₁-C₆ alkyl. In certain embodiments, R⁸ is C₁-C₃ alkyl. In certain embodiments, R⁸ is methyl, ethyl, n-propyl, or isopropyl. In certain embodiments, R⁸ is —CH₃ or —CH₂CH₃. In certain embodiments, R⁸ is methyl. In certain embodiments, R⁸ is ethyl. In certain embodiments, R⁸ is n-propyl. In certain embodiments, R⁸ is isopropyl.

In certain embodiments, R⁸ is —CN. In certain embodiments, R⁸ is C₁-C₆ alkyl-CN. In certain embodiments, R⁸ is C₁-C₃ alkyl-CN. In certain embodiments, R⁸ is —CH₂CN, —CH₂CH₂—CN, —CH₂CH₂CH₂—CN, or —C(CH₃)₂—CN. In certain embodiments, R⁸ is —CH₂CN. In certain embodiments, R⁸ is —CH₂CH₂—CN. In certain embodiments, R⁸ is —CH₂CH₂CH₂—CN. In certain embodiments, R⁸ is —C(CH₃)₂—CN.

In certain embodiments, R⁸ is C₁-C₆ alkyl-OH. In certain embodiments, R⁸ is C₁-C₃ alkyl-OH. In certain embodiments, R⁸ is —CH₂OH, —CH₂CH₂—OH, —CH₂CH₂CH₂—OH, or —C(CH₃)₂—OH. In certain embodiments, R⁸ is —CH₂OH. In certain embodiments, R⁸ is —CH₂CH₂—OH. In certain embodiments, R⁸ is —CH₂CH₂CH₂—OH. In certain embodiments, R⁸ is —C(CH₃)₂—OH.

In certain embodiments, R⁸ is C₁-C₆ haloalkyl. In certain embodiments, R⁸ is C₁-C₆ haloalkyl containing 1-7 halogen atoms. In certain embodiments, R⁸ is C₁-C₃ haloalkyl. In certain embodiments, R⁸ is C₁-C₃ haloalkyl containing 1-7 halogen atoms. In certain embodiments, R⁸ is C₁-C₃ haloalkyl containing 1-5 halogen atoms. In certain embodiments, the halogen atoms are independently selected from the group consisting of chloro, bromo, and fluoro atoms. In certain embodiments, the halogen atoms are independently selected from the group consisting of chloro and fluoro atoms. In certain embodiments, the halogen atoms are all fluoro atoms. In certain embodiments, the halogen atoms are a combination of chloro and fluoro atoms. In certain embodiments, R⁸ is —CF₃, —CCl₃, —CF₂Cl, —CFCl₂, —CHF₂, —CH₂F, —CHCl₂, —CH₂F, or —CHFCl. In certain embodiments, R⁸ is —CF₃. In certain embodiments, R⁸ is —CCl₃. In certain embodiments, R⁸ is —CF₂Cl. In certain embodiments, R⁸ is —CFCl₂. In certain embodiments, R⁸ is —CHF₂. In certain embodiments, R⁸ is —CH₂F. In certain embodiments, R⁸ is —CHCl₂. In certain embodiments, R⁸ is —CH₂F. In certain embodiments, R⁸ is —CHFCl.

In certain embodiments, R⁸ is oxo.

In certain embodiments, R⁸ is —O(C₁-C₆ alkyl). In certain embodiments, R⁸ is —O—(C₁-C₃ alkyl). In certain embodiments, R⁸ is —O(methyl), —O(ethyl), —O(n-propyl), or —O(isopropyl). In certain embodiments, R⁸ is —OCH₃ or —OCH₂CH₃. In certain embodiments, R⁸ is —OCH₃. In certain embodiments, R⁸ is —OCH₂CH₃. In certain embodiments, R⁸ is —OCH₂CH₂CH₃. In certain embodiments, R⁸ is —OCH(CH₃)₂.

In certain embodiments, two R⁸ groups are taken together with the carbon atom or atoms to which they are attached to form a spiro or fused C₃-C₅ cycloalkyl or a 3- to 5-membered heterocyclyl. In certain embodiments, two R⁸ groups are taken together with the carbon atom or atoms to which they are attached to form a spiro or fused cyclopropyl or oxetanyl.

In certain embodiments, two R⁸ groups are taken together with the carbon atom or atoms to which they are attached to form a spiro or fused C₃-C₅ cycloalkyl. In certain embodiments, two R⁸ groups are taken together with the carbon atom to which they are attached to form a spiro C₃-C₅ cycloalkyl. In certain embodiments, two R⁸ groups are taken together with the carbon atom to which they are attached to form a spiro cyclopropyl. In certain embodiments, two R⁸ groups are taken together with the carbon atom to which they are attached to form a spiro cyclobutyl. In certain embodiments, two R⁸ groups are taken together with the carbon atom to which they are attached to form a spiro cyclopentyl. In certain embodiments, two R⁸ groups are taken together with the carbon atoms to which they are attached to form a fused C₃-C₅ cycloalkyl. In certain embodiments, two R⁸ groups are taken together with the carbon atoms to which they are attached to form a fused cyclopropyl. In certain embodiments, two R⁸ groups are taken together with the carbon atoms to which they are attached to form a fused cyclobutyl. In certain embodiments, two R⁸ groups are taken together with the carbon atoms to which they are attached to form a fused cyclopentyl. In certain embodiments, two R⁸ groups are taken together with the carbon atom or atoms to which they are attached to form a spiro or fused cyclopropyl.

In certain embodiments, two R⁸ groups are taken together with the carbon atom or atoms to which they are attached to form a spiro or fused 3- to 5-membered heterocyclyl. In certain embodiments, two R⁸ groups are taken together with the carbon atom to which they are attached to form a spiro 3- to 5-membered heterocyclyl. In certain embodiments, two R⁸ groups are taken together with the carbon atom to which they are attached to form a spiro oxetanyl. In certain embodiments, two R⁸ groups are taken together with the carbon atoms to which they are attached to form a fused 3- to 5-membered heterocyclyl. In certain embodiments, two R⁸ groups are taken together with the carbon atoms to which they are attached to form a fused oxetanyl. In certain embodiments, two R⁸ groups are taken together with the carbon atom or atoms to which they are attached to form a spiro or fused oxetanyl. In certain embodiments, the heterocyclyl contains 1-3 heteroatoms selected from the group consisting of nitrogen, oxygen, and sulfur. In certain embodiments, the heterocyclyl contains one nitrogen atom. In certain embodiments, the heterocyclyl contains two nitrogen atoms. In certain embodiments, the heterocyclyl contains one oxygen atom. In certain embodiments, the heterocyclyl contains two oxygen atoms. In certain embodiments, the heterocyclyl contains one oxygen atom and one nitrogen atom. In certain embodiments, the heterocyclyl contains one sulfur atom. In certain embodiments, the heterocyclyl contains one nitrogen atom and one sulfur atom. In certain embodiments, two R⁸ groups are taken together with the carbon atom to which they are attached to form a spiro aziridinyl, oxiranyl, oxetanyl, azetidinyl, tetrahydrofuranyl, dioxolanyl, pyrrolidinyl, pyrazolidinyl, or isoxazolidinyl. In certain embodiments, two R⁸ groups are taken together with the carbon atom to which they are attached to form a spiro aziridinyl. In certain embodiments, two R⁸ groups are taken together with the carbon atom to which they are attached to form a spiro oxiranyl. In certain embodiments, two R⁸ groups are taken together with the carbon atom to which they are attached to form a spiro oxetanyl. In certain embodiments, two R⁸ groups are taken together with the carbon atom to which they are attached to form a spiro azetidinyl. In certain embodiments, two R⁸ groups are taken together with the carbon atom to which they are attached to form a spiro tetrahydrofuranyl. In certain embodiments, two R⁸ groups are taken together with the carbon atom to which they are attached to form a spiro dioxolanyl. In certain embodiments, two R⁸ groups are taken together with the carbon atom to which they are attached to form a spiro pyrrolidinyl. In certain embodiments, two R⁸ groups are taken together with the carbon atom to which they are attached to form a spiro pyrazolidinyl. In certain embodiments, two R⁸ groups are taken together with the carbon atom to which they are attached to form a spiro isoxazolidinyl. In certain embodiments, two R⁸ groups are taken together with the carbon atoms to which they are attached to form a fused oxetanyl, azetidinyl, tetrahydrofuranyl, dioxolanyl, pyrrolidinyl, pyrazolidinyl, or isoxazolidinyl. In certain embodiments, two R⁸ groups are taken together with the carbon atoms to which they are attached to form a fused oxetanyl. In certain embodiments, two R⁸ groups are taken together with the carbon atoms to which they are attached to form a fused azetidinyl. In certain embodiments, two R⁸ groups are taken together with the carbon atoms to which they are attached to form a fused tetrahydrofuranyl. In certain embodiments, two R⁸ groups are taken together with the carbon atoms to which they are attached to form a fused dioxolanyl. In certain embodiments, two R⁸ groups are taken together with the carbon atoms to which they are attached to form a fused pyrrolidinyl. In certain embodiments, two R⁸ groups are taken together with the carbon atoms to which they are attached to form a fused pyrazolidinyl. In certain embodiments, two R⁸ groups are taken together with the carbon atoms to which they are attached to form a fused isoxazolidinyl.

In certain embodiments, X is

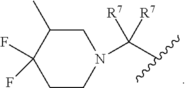

In certain embodiments, X is

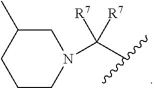

In certain embodiments, X is

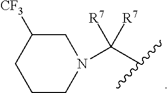

In certain embodiments, X is

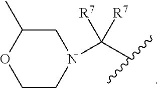

In certain embodiments, X is

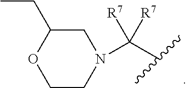

In certain embodiments, X is

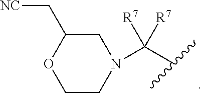

In any of these embodiments, both R⁷ groups can be hydrogen (H). In any of these embodiments, one R⁷ group can be hydrogen and one R⁷ group can be —CH₃. In any of these embodiments, both R⁷ groups can be —CH₃.

In certain embodiments, the compound is of Formula (I-A) or (I-B)

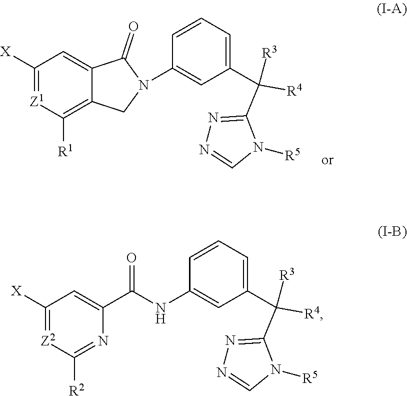

wherein R¹, R², R³, R⁴, R⁵, Z¹, Z², and X are as described for the compound of Formula (I).

In certain embodiments, the compound is of Formula (I-a) or (I-b)

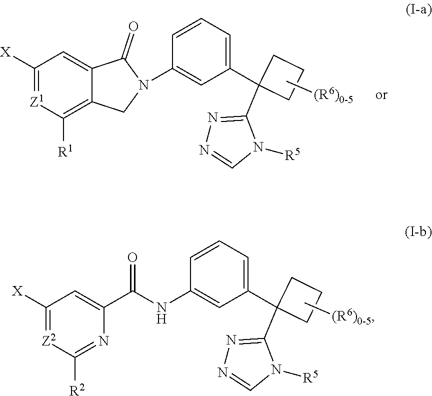

wherein R¹, R², R⁵, R⁶, Z¹, Z², and X are as described for the compound of Formula (I).

In certain embodiments, the compound is of Formula (I-C), (I-D), (I-E), (I-F), (I-G), (I-H), or (I-J)

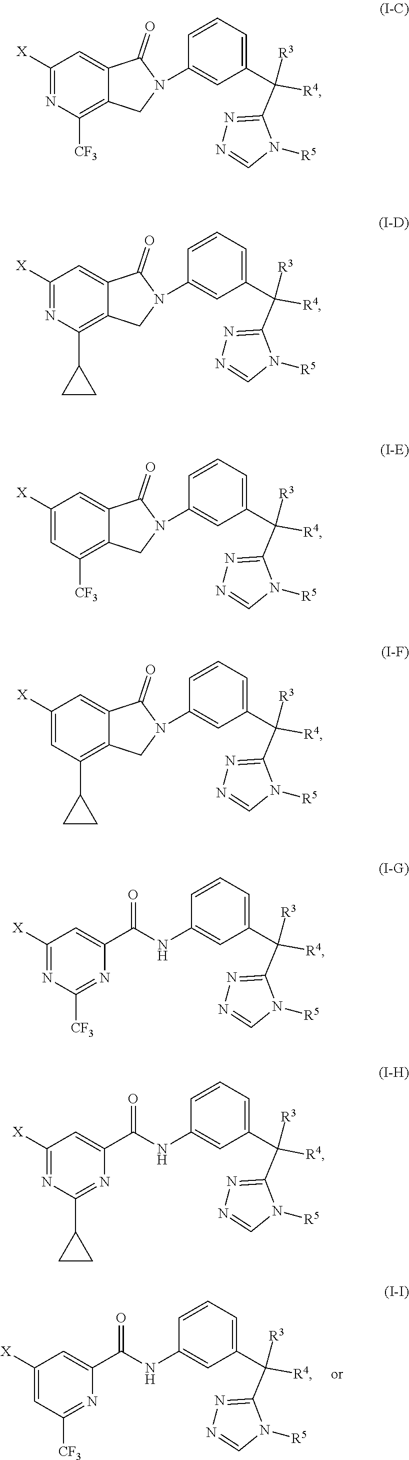

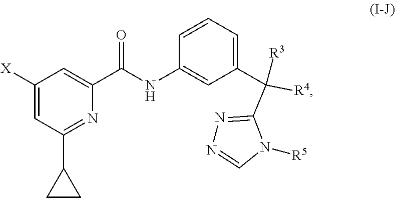

wherein R³, R⁴, R⁵, and X are as described for the compound of Formula (I).

In certain embodiments, the compound is of Formula (II-A), (II-B), (II-C), (II-D), (II-E), (II-F), (II-G), or (II-H)

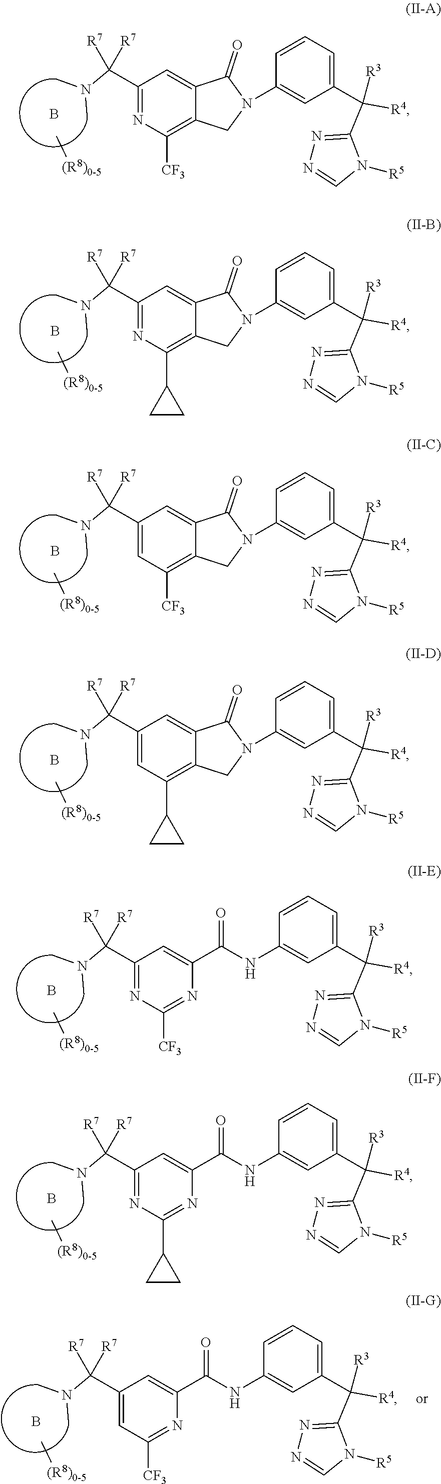

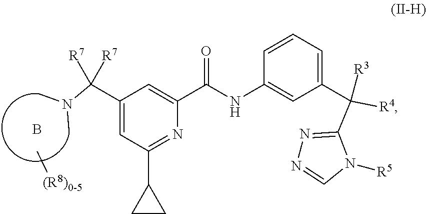

wherein R³, R⁴, R⁵, R⁷, R⁸ and the Ring B moiety are as described for the compound of Formula (I).

In certain embodiments, the compound is of (III-A), (III-B), (III-C), (III-D), (III-E), (III-F), (III-G), or (III-H)

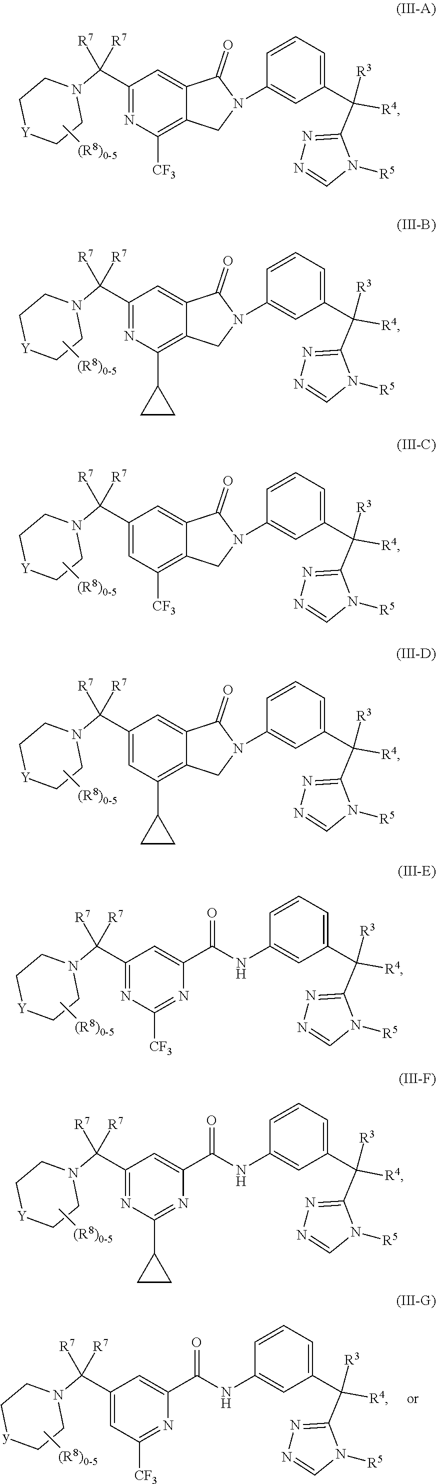

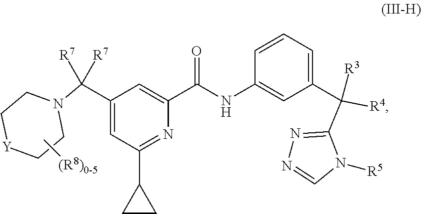

wherein R³, R⁴, R⁵, R⁷, R⁸, and Y are as described for the compound of Formula (I).

In certain embodiments, the compound is of (IV-A), (IV-B), (IV-C), (IV-D), (IV-E), (IV-F), (IV-G), or (IV-H)

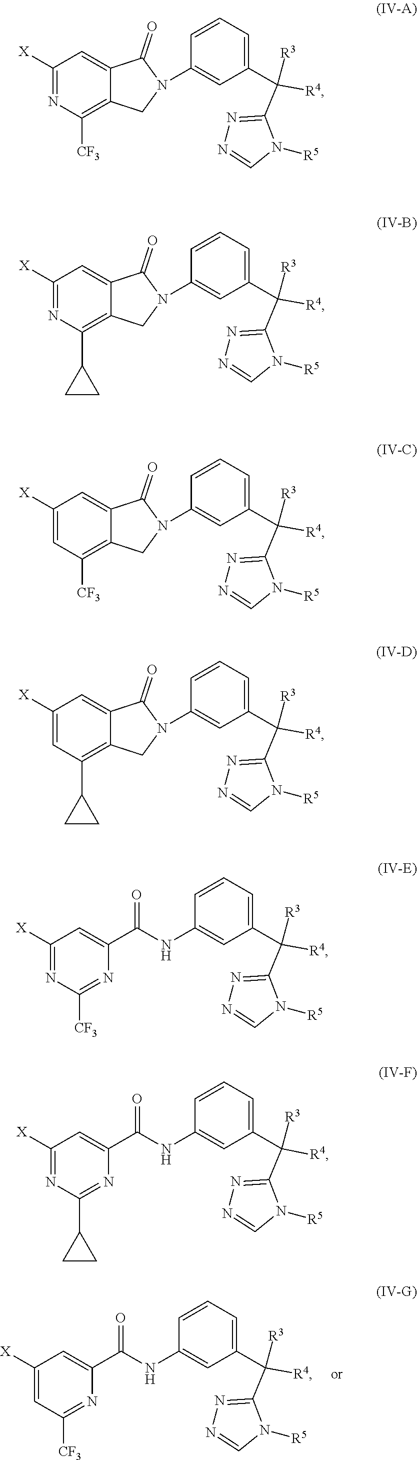

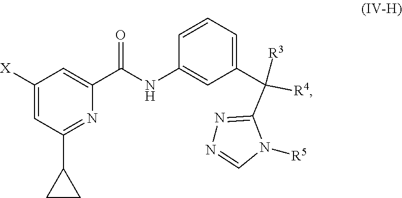

wherein R³, R⁴, and R⁵ are as described for the compound of Formula (I), and X is hydrogen, C₁-C₆ alkyl, C₁-C₆ haloalkyl, C₁-C₆ alkyl-OH, C₁-C₆ alkyl-CN, or C₃-C₆ cycloalkyl optionally substituted by 1-5 R⁸ groups. In certain embodiments, X is hydrogen. In certain embodiments, X is C₁-C₆ alkyl. In certain embodiments, X is C₁-C₆ haloalkyl. In certain embodiments, X is C₁-C₆ alkyl-OH. In certain embodiments, X is C₁-C₆ alkyl-CN. In certain embodiments, X is C₃-C₆ cycloalkyl optionally substituted by 1-5 R⁸ groups.

In certain embodiments, the compound is selected from Table 1, or a pharmaceutically acceptable stereoisomer, tautomer, or salt thereof.

TABLE 1
Representative Compounds of This Disclosure

| Cmpd | |
|---|---|
| No. | Structure |
| 1 | 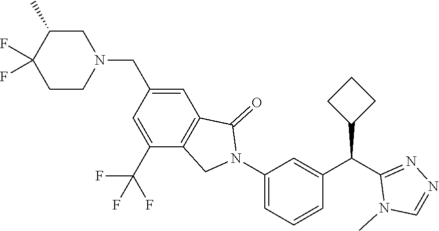 [SMILES: C[C@@H]1CN(Cc2cc3c(c(C(F)(F)F)c2)CN(c2cccc([C@H](c4nncn4C)C4CCC4)c2)C3=O)CCC1(F)F] |
| 2 | 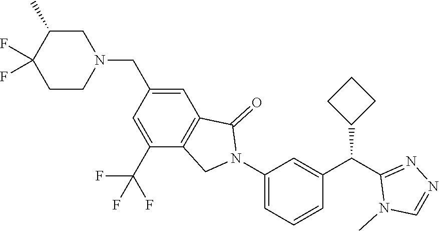 [SMILES: C[C@@H]1CN(Cc2cc3c(c(C(F)(F)F)c2)CN(c2cccc([C@@H](c4nncn4C)C4CCC4)c2)C3=O)CCC1(F)F] |
| 3 | 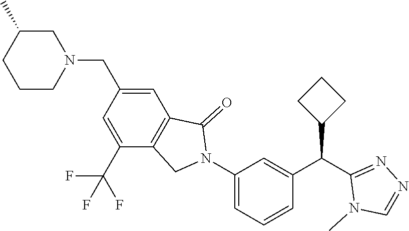 [SMILES: C[C@H]1CCCN(Cc2cc3c(c(C(F)(F)F)c2)CN(c2cccc([C@H](c4nncn4C)C4CCC4)c2)C3=O)C1] |
| 4 | 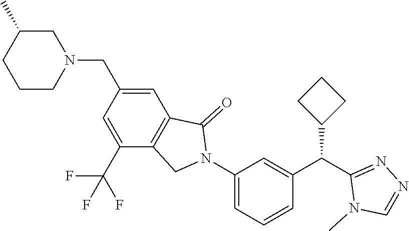 [SMILES: C[C@H]1CCCN(Cc2cc3c(c(C(F)(F)F)c2)CN(c2cccc([C@@H](c4nncn4C)C4CCC4)c2)C3=O)C1] |
| 5 | 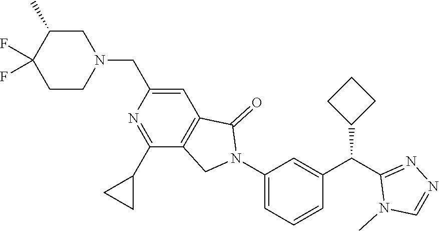 [SMILES: C[C@@H]1CN(Cc2cc3c(c(C4CC4)n2)CN(c2cccc([C@@H](c4nncn4C)C4CCC4)c2)C3=O)CCC1(F)F] |
| 6 | 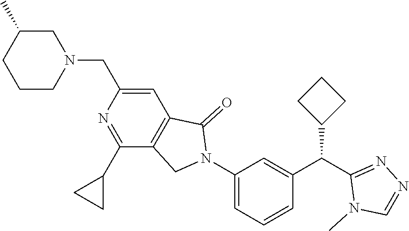 [SMILES: C[C@H]1CCCN(Cc2cc3c(c(C4CC4)n2)CN(c2cccc([C@@H](c4nncn4C)C4CCC4)c2)C3=O)C1] |
| 7 | 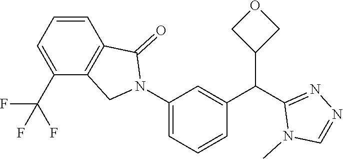 [SMILES: Cn1cnnc1C(c1cccc(N2Cc3c(cccc3C(F)(F)F)C2=O)c1)C1COC1] |
| 8 | 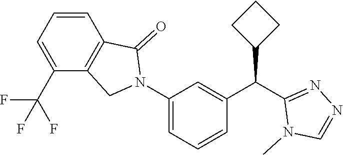 [SMILES: Cn1cnnc1[C@@H](c1cccc(N2Cc3c(cccc3C(F)(F)F)C2=O)c1)C1CCC1] |
| 9 | 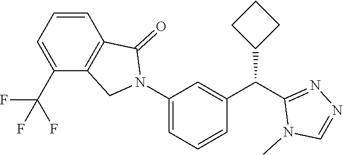 [SMILES: Cn1cnnc1[C@H](c1cccc(N2Cc3c(cccc3C(F)(F)F)C2=O)c1)C1CCC1] |
| 10 | 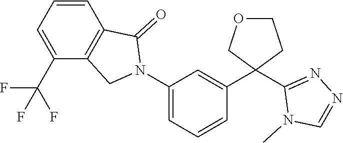 [SMILES: Cn1cnnc1C1(c2cccc(N3Cc4c(cccc4C(F)(F)F)C3=O)c2)CCOC1] |
| 11 | 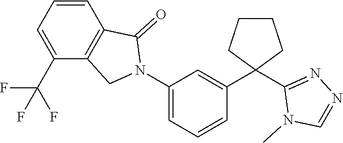 [SMILES: Cn1cnnc1C1(c2cccc(N3Cc4c(cccc4C(F)(F)F)C3=O)c2)CCCC1] |
| 12 | 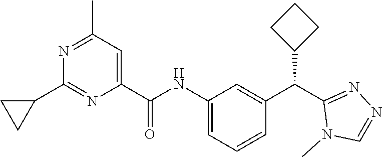 [SMILES: Cc1cc(C(=O)Nc2cccc([C@@H](c3nncn3C)C3CCC3)c2)nc(C2CC2)n1] |
| 13 | 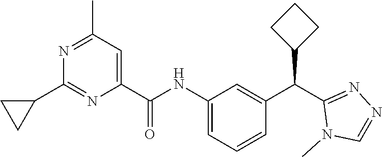 [SMILES: Cc1cc(C(=O)Nc2cccc([C@H](c3nncn3C)C3CCC3)c2)nc(C2CC2)n1] |
| 14 | 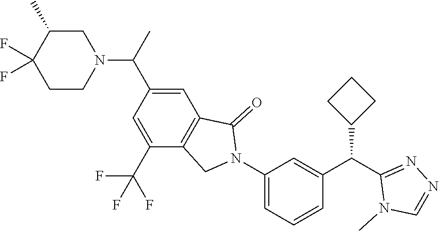 [SMILES: CC(c1cc2c(c(C(F)(F)F)c1)CN(c1cccc([C@@H](c3nncn3C)C3CCC3)c1)C2=O)N1CCC(F)(F)[C@H](C)C1] |
| 15 | 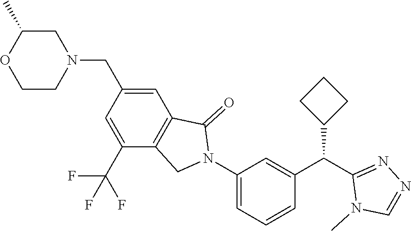 [SMILES: C[C@@H]1CN(Cc2cc3c(c(C(F)(F)F)c2)CN(c2cccc([C@@H](c4nncn4C)C4CCC4)c2)C3=O)CCO1] |
| 16 | 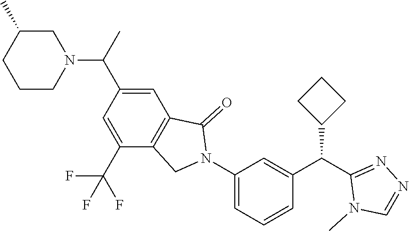 [SMILES: CC(c1cc2c(c(C(F)(F)F)c1)CN(c1cccc([C@@H](c3nncn3C)C3CCC3)c1)C2=O)N1CCC[C@H](C)C1] |
| 17 | 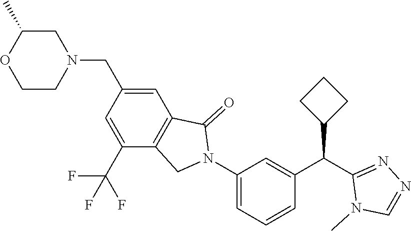 [SMILES: C[C@@H]1CN(Cc2cc3c(c(C(F)(F)F)c2)CN(c2cccc([C@H](c4nncn4C)C4CCC4)c2)C3=O)CCO1] |
| 18 | 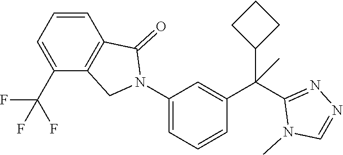 [SMILES: Cn1cnnc1C(C)(c1cccc(N2Cc3c(cccc3C(F)(F)F)C2=O)c1)C1CCC1] |
| 19 | 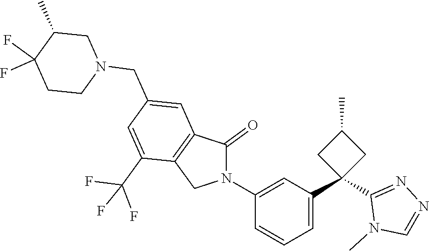 [SMILES: C[C@@H]1CN(Cc2cc3c(c(C(F)(F)F)c2)CN(c2cccc([C@]4(c5nncn5C)C[C@@H](C)C4)c2)C3=O)CCC1(F)F] |
| 20 | 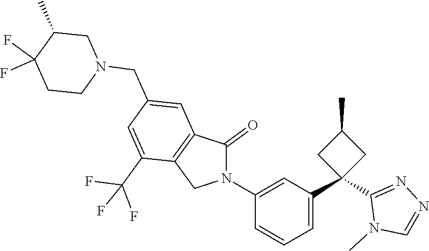 [SMILES: C[C@@H]1CN(Cc2cc3c(c(C(F)(F)F)c2)CN(c2cccc([C@]4(c5nncn5C)C[C@H](C)C4)c2)C3=O)CCC1(F)F] |
| 21 | 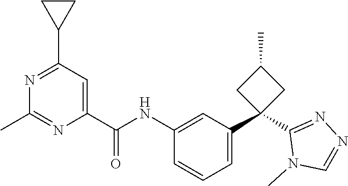 [SMILES: Cc1nc(C(=O)Nc2cccc([C@]3(c4nncn4C)C[C@@H](C)C3)c2)cc(C2CC2)n1] |
| 22 | 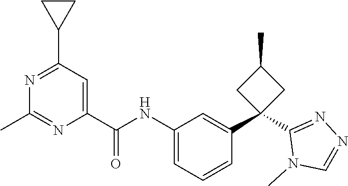 [SMILES: Cc1nc(C(=O)Nc2cccc([C@]3(c4nncn4C)C[C@H](C)C3)c2)cc(C2CC2)n1] |
| 23 | 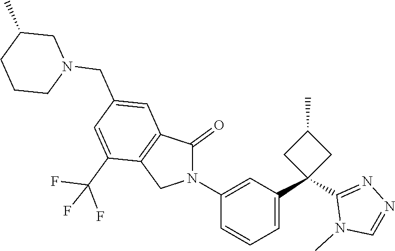 [SMILES: C[C@H]1CCCN(Cc2cc3c(c(C(F)(F)F)c2)CN(c2cccc([C@]4(c5nncn5C)C[C@@H](C)C4)c2)C3=O)C1] |
| 24 | 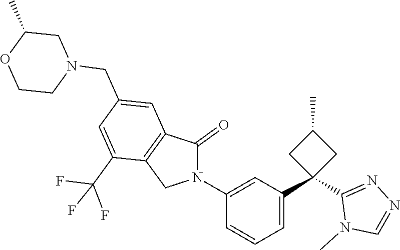 [SMILES: C[C@@H]1CN(Cc2cc3c(c(C(F)(F)F)c2)CN(c2cccc([C@]4(c5nncn5C)C[C@@H](C)C4)c2)C3=O)CCO1] |
| 25 | 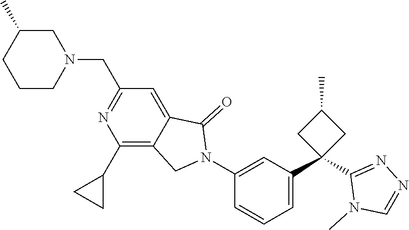 [SMILES: C[C@H]1CCCN(Cc2cc3c(c(C4CC4)n2)CN(c2cccc([C@]4(c5nncn5C)C[C@@H](C)C4)c2)C3=O)C1] |
| 26 | 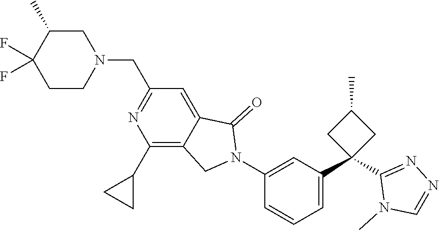 [SMILES: C[C@@H]1CN(Cc2cc3c(c(C4CC4)n2)CN(c2cccc([C@]4(c5nncn5C)C[C@@H](C)C4)c2)C3=O)CCC1(F)F] |
| 27 | 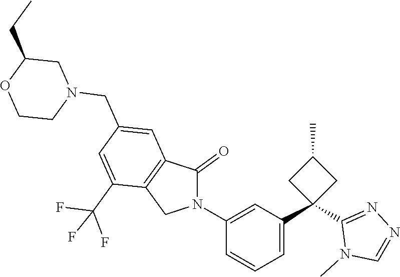 [SMILES: CC[C@H]1CN(Cc2cc3c(c(C(F)(F)F)c2)CN(c2cccc([C@]4(c5nncn5C)C[C@@H](C)C4)c2)C3=O)CCO1] |
| 28 | 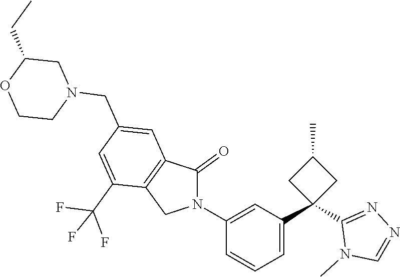 [SMILES: CC[C@@H]1CN(Cc2cc3c(c(C(F)(F)F)c2)CN(c2cccc([C@]4(c5nncn5C)C[C@@H](C)C4)c2)C3=O)CCO1] |
| 29 | 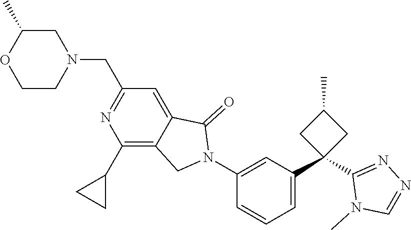 [SMILES: C[C@@H]1CN(Cc2cc3c(c(C4CC4)n2)CN(c2cccc([C@]4(c5nncn5C)C[C@@H](C)C4)c2)C3=O)CCO1] |
| 30 | 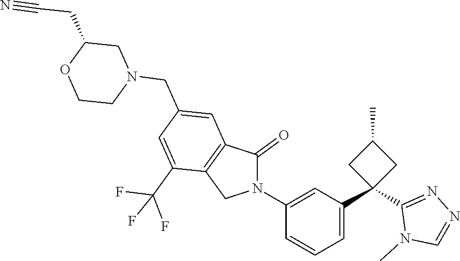 [SMILES: Cn1cnnc1[C@]1(c2cccc(N3Cc4c(cc(CN5CCO[C@H](CC#N)C5)cc4C(F)(F)F)C3=O)c2)C[C@H](C)C1] |
| 31 | 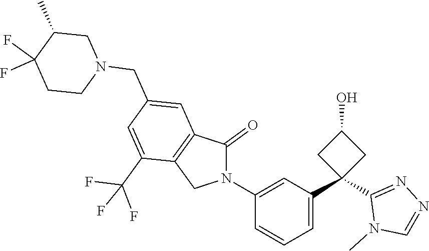 [SMILES: C[C@@H]1CN(Cc2cc3c(c(C(F)(F)F)c2)CN(c2cccc([C@]4(c5nncn5C)C[C@@H](O)C4)c2)C3=O)CCC1(F)F] |
| 32 | 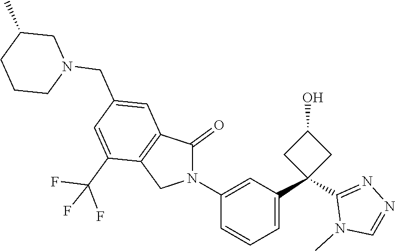 [SMILES: C[C@H]1CCCN(Cc2cc3c(c(C(F)(F)F)c2)CN(c2cccc([C@]4(c5nncn5C)C[C@@H](O)C4)c2)C3=O)C1] |
| 33 | 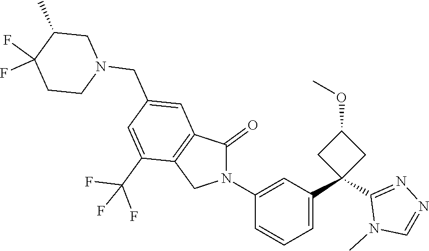 [SMILES: CO[C@H]1C[C@@](c2cccc(N3Cc4c(cc(CN5CCC(F)(F)[C@H](C)C5)cc4C(F)(F)F)C3=O)c2)(c2nncn2C)C1] |
| 34 | 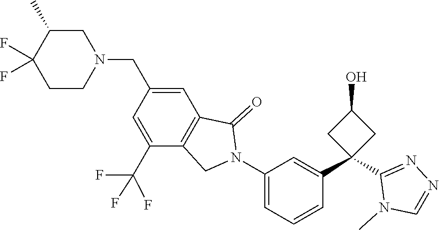 [SMILES: C[C@@H]1CN(Cc2cc3c(c(C(F)(F)F)c2)CN(c2cccc([C@]4(c5nncn5C)C[C@H](O)C4)c2)C3=O)CCC1(F)F] |
| 35 | 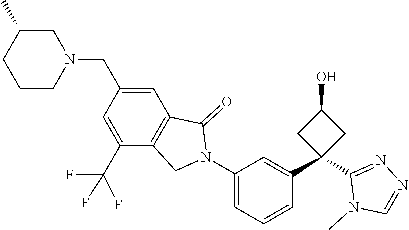 [SMILES: C[C@H]1CCCN(Cc2cc3c(c(C(F)(F)F)c2)CN(c2cccc([C@]4(c5nncn5C)C[C@H](O)C4)c2)C3=O)C1] |
| 36 | 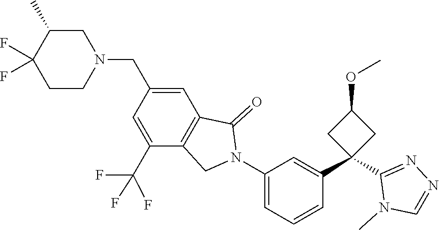 [SMILES: CO[C@H]1C[C@](c2cccc(N3Cc4c(cc(CN5CCC(F)(F)[C@H](C)C5)cc4C(F)(F)F)C3=O)c2)(c2nncn2C)C1] |
| 37 | 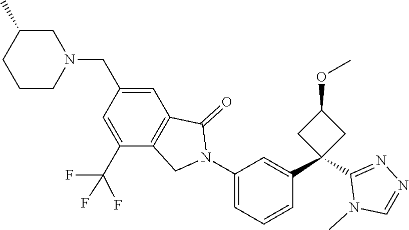 [SMILES: CO[C@H]1C[C@](c2cccc(N3Cc4c(cc(CN5CCC[C@H](C)C5)cc4C(F)(F)F)C3=O)c2)(c2nncn2C)C1] |
| 38 | 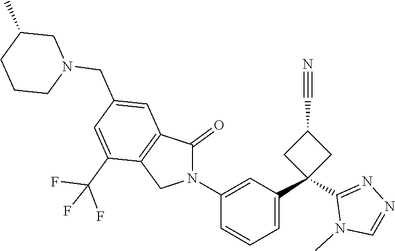 [SMILES: C[C@H]1CCCN(Cc2cc3c(c(C(F)(F)F)c2)CN(c2cccc([C@]4(c5nncn5C)C[C@@H](C#N)C4)c2)C3=O)C1] |
| 39 | 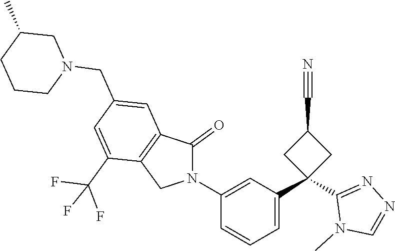 [SMILES: C[C@H]1CCCN(Cc2cc3c(c(C(F)(F)F)c2)CN(c2cccc([C@]4(c5nncn5C)C[C@H](C#N)C4)c2)C3=O)C1] |
| 40 | 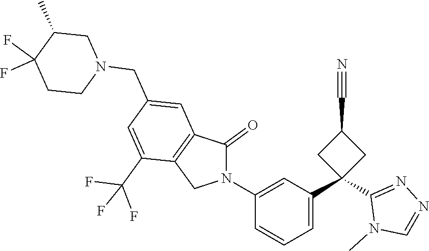 [SMILES: C[C@@H]1CN(Cc2cc3c(c(C(F)(F)F)c2)CN(c2cccc([C@]4(c5nncn5C)C[C@H](C#N)C4)c2)C3=O)CCC1(F)F] |
| 41 | 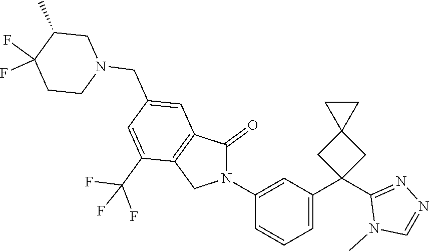 [SMILES: C[C@@H]1CN(Cc2cc3c(c(C(F)(F)F)c2)CN(c2cccc(C4(c5nncn5C)CC5(CC5)C4)c2)C3=O)CCC1(F)F] |
| 42 | 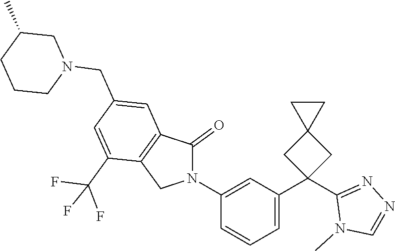 [SMILES: C[C@H]1CCCN(Cc2cc3c(c(C(F)(F)F)c2)CN(c2cccc(C4(c5nncn5C)CC5(CC5)C4)c2)C3=O)C1] |
| 43 | 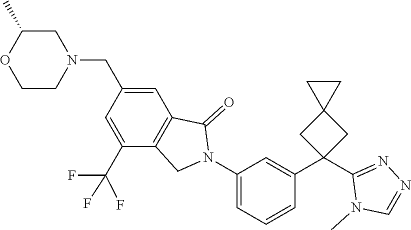 [SMILES: C[C@@H]1CN(Cc2cc3c(c(C(F)(F)F)c2)CN(c2cccc(C4(c5nncn5C)CC5(CC5)C4)c2)C3=O)CCO1] |
| 44 | 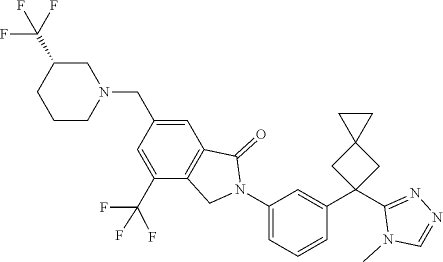 [SMILES: Cn1cnnc1C1(c2cccc(N3Cc4c(cc(CN5CCC[C@H](C(F)(F)F)C5)cc4C(F)(F)F)C3=O)c2)CC2(CC2)C1] |
| 45 | 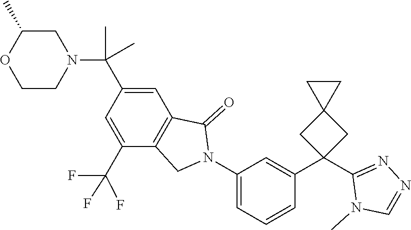 [SMILES: C[C@@H]1CN(C(C)(C)c2cc3c(c(C(F)(F)F)c2)CN(c2cccc(C4(c5nncn5C)CC5(CC5)C4)c2)C3=O)CCO1] |
| 46 | 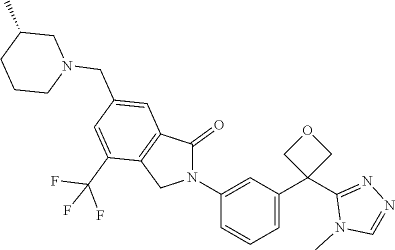 [SMILES: C[C@H]1CCCN(Cc2cc3c(c(C(F)(F)F)c2)CN(c2cccc(C4(c5nncn5C)COC4)c2)C3=O)C1] |
| 47 | 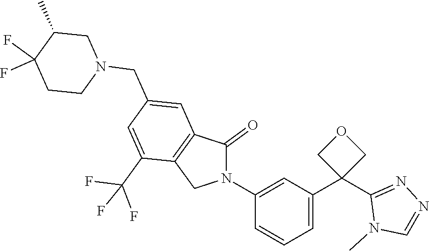 [SMILES: C[C@@H]1CN(Cc2cc3c(c(C(F)(F)F)c2)CN(c2cccc(C4(c5nncn5C)COC4)c2)C3=O)CCC1(F)F] |
| 48 | 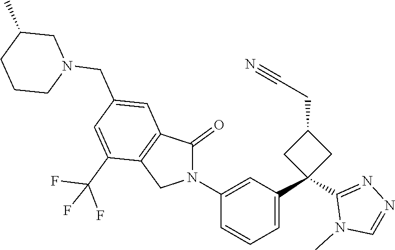 [SMILES: C[C@H]1CCCN(Cc2cc3c(c(C(F)(F)F)c2)CN(c2cccc([C@]4(c5nncn5C)C[C@@H](CC#N)C4)c2)C3=O)C1] |
| 49 | 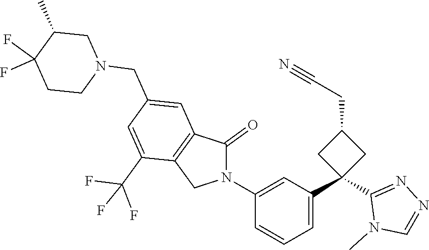 [SMILES: C[C@@H]1CN(Cc2cc3c(c(C(F)(F)F)c2)CN(c2cccc([C@]4(c5nncn5C)C[C@@H](CC#N)C4)c2)C3=O)CCC1(F)F] |
| 50 | 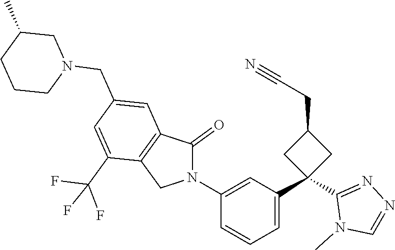 [SMILES: C[C@H]1CCCN(Cc2cc3c(c(C(F)(F)F)c2)CN(c2cccc([C@]4(c5nncn5C)C[C@H](CC#N)C4)c2)C3=O)C1] |
| 51 | 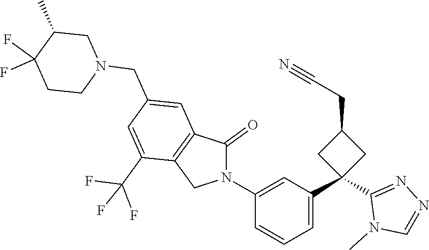 [SMILES: C[C@@H]1CN(Cc2cc3c(c(C(F)(F)F)c2)CN(c2cccc([C@]4(c5nncn5C)C[C@H](CC#N)C4)c2)C3=O)CCC1(F)F] |
| 52 | 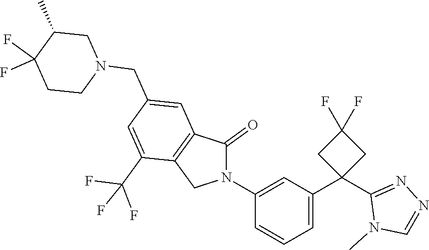 [SMILES: C[C@@H]1CN(Cc2cc3c(c(C(F)(F)F)c2)CN(c2cccc(C4(c5nncn5C)CC(F)(F)C4)c2)C3=O)CCC1(F)F] |
| 53 | 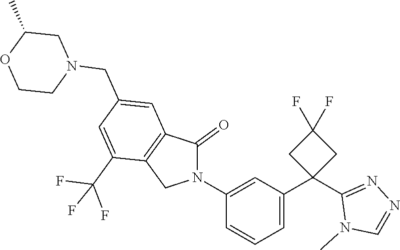 [SMILES: C[C@@H]1CN(Cc2cc3c(c(C(F)(F)F)c2)CN(c2cccc(C4(c5nncn5C)CC(F)(F)C4)c2)C3=O)CCO1] |

### 4. TLR7 Agonists

In the combinations provided herein, the TLR7 agonist can be any TLR7 agonist known to the practitioner of skill. In certain embodiments, the TLR7 agonist is a small molecule. In certain embodiments, the TLR7 agonist is an antibody. In certain embodiments, the TLR7 agonist is an oligonucleotide. In certain embodiments, the TLR7 agonist binds TLR7. In certain embodiments, the TLR7 agonist increases TLR7 activity. In certain embodiments, the TLR7 agonist is selected from the group consisting of resiquimod (Galderma), imiquimod (Aldara), and gardiquimod (Invivogen). In certain embodiments, the TLR7 agonist is resiquimod (Galderma). In certain embodiments, the TLR7 agonist is imiquimod (Aldara). In certain embodiments, the TLR7 agonist is gardiquimod (Invivogen).

### 5. TLR9 Agonists

In the combinations provided herein, the TLR9 agonist can be any TLR9 agonist known to the practitioner of skill. In certain embodiments, the TLR9 agonist is a small molecule. In certain embodiments, the TLR9 agonist is an antibody. In certain embodiments, the TLR9 agonist is an oligonucleotide. In certain embodiments, the TLR9 agonist binds TLR9. In certain embodiments, the TLR9 agonist increases TLR9 activity. In certain embodiments, the TLR9 agonist is selected from the group consisting of lefitolimod (Mologen), tilsotolimod (Idera), and SD-101 (Dynavax). In certain embodiments, the TLR9 agonist is lefitolimod (Mologen). In certain embodiments, the TLR9 agonist is tilsotolimod (Idera). In certain embodiments, the TLR9 agonist is SD-101 (Dynavax). In certain embodiments, the TLR9 agonist is any described in U.S. Pat. Nos. 7,745,606, 10,751,412, or 9,993,495, each of which are incorporated by reference herein in their entireties.

In particular embodiments, the TLR9 agonist is a CpG oligonucleotide. In some embodiments, the oligonucleotide is at least (lower limit) 12, 13, 14, 15, 16, 17, 18, 19, 20, 21, 22, 23, 24, 25, 26, 27, 28, 29, 30, 32, 34, 36, 38, 40, 50, 60, 70, 80, or 90 bases in length. In some embodiments, the oligonucleotide is at most (upper limit) 100, 90, 80, 70, 60, 50, 49, 48, 47, 46, 45, 44, 43, 42, 41, 40, 39, 38, 37, 36, 35, 34, 33, 32, 31, or 30 bases in length. In certain embodiments, the oligonucleotide comprises at least one palindromic sequence. In some embodiments, the at least one palindromic sequence is 8 to 97 bases in length, 8 to 50 bases in length, or 8 to 32 bases in length. In some embodiments, the at least one palindromic sequence is at least (lower limit) 8, 10, 12, 14, 16, 18, 20, 22, 24, 26, 28, or 30 bases in length. In some embodiments, the at least one palindromic sequence is at most (upper limit) 50, 48, 46, 44, 42, 40, 38, 36, 34, 32, 30, 28, 26, 24, 22, 20, 18, 16, 14, 12, or 10 bases in length. In one embodiment, the oligonucleotide is an oligodeoxynucleotide. In one embodiment, one or more of the internucleotide linkages of the CpG oligonucleotides are modified linkages. In one embodiment, one or more of the internucleotide linkages of CpG oligonucleotides are phosphorothioate (PS) linkages. In one embodiment, all of the internucleotide linkages of CpG oligonucleotide are phosphorothioate (PS) linkages. A phosphorothioate backbone refers to all of the internucleotide linkages of the CpG oligonucleotide being phosphorothioate (PS) linkages.

The CpG oligonucleotides and SEQ ID NOS:1-15 discussed herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (i.e., organic amines) such as N-Me-D-glutamine, N-[1-(2,3-dioleoyloxy)propyl]-N,N,N-trimethylammonium chloride, choline, tromethamine, dicyclohexylamines, t-butyl amines, and salts with amino acids such as arginine, lysine, and the like. In one embodiment, the CpG type oligonucleotides are in the ammonium, sodium, lithium, or potassium salt form. In one embodiment, the CpG type oligonucleotides are in the sodium salt form. The CpG oligonucleotides may be provided in a pharmaceutical solution comprising a pharmaceutically acceptable excipient. Alternatively, the CpG oligonucleotide may be provided as a lyophilized solid, which is subsequently reconstituted in sterile water, saline or a pharmaceutically acceptable buffer before administration.

In certain embodiments, the TLR9 agonist is an oligodeoxynucleotide 12 to 100 bases in length comprising the sequence 5′-Nₓ(TCG(N_{q}))_{y}N_{w}(X¹X²CGX²′X¹′(CG)ₚ)_{z}Nᵥ-3′ (SEQ ID NO:1), wherein each N is a nucleoside; x is 0, 1, 2, or 3; y is 1, 2, 3, or 4; w is 0, 1, or 2; p is zero or one; q is 0, 1 or 2; v is an integer from zero to eighty-nine; and z is an integer from one to twenty; X¹ and X¹″ are self-complementary deoxynucleosides; X² and X^{2′} are self-complementary deoxynucleosides; the 5′-T of (TCG(N_{q})_{y} is 0-3 bases from the 5′ end of the oligodeoxynucleotide; wherein the oligodeoxynucleotide comprises a palindromic sequence at least eight bases in length including the first X¹X²CGX²′X¹′(CG)ₚ(SEQ ID NO:2) of (X¹X²CGX²′X¹′(CG)ₚ)_{z} (SEQ ID NO:3). In certain embodiments, x is zero; y is one; w is zero; p is zero or one; q is zero, one, or two; v is an integer from zero to twenty; and z is one, two, three, or four. In certain embodiments, the oligodeoxynucleotide consists of 5′-TCGN_{q}(X¹X²CGX²′X¹′CG)_{z}Nᵥ-3′ (SEQ ID NO:4) wherein q is one, two, three, four, or five; v is an integer from zero to twenty; and z is one, two, three, or four. In certain embodiments, the oligodeoxynucleotide consists of 5′-TCGN_{q}TTCGAACGTTCGAACGTTNₛ-3′ (SEQ ID NO:5) wherein q is one, two, three, four, or five; and s is an integer from zero to twenty. In certain embodiments, the oligodeoxynucleotide consists of a sequence selected from the group consisting of SEQ ID NOS:6-18. In certain embodiments, the oligodeoxynucleotide consists of a sequence according to SEQ ID NO:10. In certain embodiments, the oligodeoxynucleotide is any described in U.S. Pat. Nos. 7,745,606, 8,158,768, 8,871,732 and 10,751,412, each of which are incorporated by reference herein in their entireties.

In certain embodiments, the oligodeoxynucleotide comprises at least one phosphorothioate. In certain embodiments, the oligodeoxynucleotide comprises a phosphorothioate backbone.

In another embodiment, the TLR9 agonist CpG oligonucleotide is selected from the group consisting of

| | |
|---|---|
| SEQ ID NO: 6 | 5′-TCGTCGAACGTTCGAGATGAT-3′ |
| SEQ ID NO: 7 | 5′-TCGTTCGAACGTTCGAACGTTCGAA-3′ |
| SEQ ID NO: 8 | 5′-TCGAACGTTCGAACGTTCGAACGTT-3′ |
| SEQ ID NO: 9 | 5′-TCGAACGTTCGAACGTTCGAATTTT-3′ |
| SEQ ID NO: 10 | 5′-TCGAACGTTCGAACGTTCGAACGTTCGAAT-3′ |
| SEQ ID NO: 11 | 5′-TCGTAACGTTCGAACGTTCGAACGTTA-3′ |
| SEQ ID NO: 12 | 5′-TCGTAACGTTCGAACGTTCGAACGTT-3′ |
| SEQ ID NO: 13 | 5′-TCGTAACGTTCGAACGTTCGAACGT-3′ |
| SEQ ID NO: 14 | 5′-TCGTAACGTTCGAACGTTCGAACG-3′ |
| SEQ ID NO: 15 | 5′-TCGTAACGTTCGAACGTTCGAAC-3′ |
| SEQ ID NO: 16 | 5′-TCGTAACGTTCGAACGTTCGAA-3′ |
| SEQ ID NO: 17 | 5′-TGACTGTGAACGTTCGAGATGA-3′ |
| SEQ ID NO: 18 | 5′-TCGTTCGAACGTTCGAACGTTCG-3′ |

It is understood that, with respect to formulae or sequence motifs described herein, any and all parameters are independently selected. For example, if x=0−2, y may be independently selected regardless of the value of x (or any other selectable parameter in a formula), as long as the total oligonucleotide length limitation is met.

Additional CpG-C oligonucleotides having sequences encompassed by the motifs of the present disclosure are suitable for use in the methods and medicaments disclosed herein. A plurality of additional CpG-C oligonucleotides having sequences encompassed by the motifs of the present disclosure are described in U.S. Pat. Nos. 7,745,606, 8,158,768, and 8,871,732 to Dynavax Technologies Corporation. These sequences are hereby incorporated by reference herein.

CpG oligonucleotides have been described in the art and their activity may be readily determined using standard assays, which measure various aspects of immune responses (e.g., cytokine secretion, antibody production, NK cell activation, B cell proliferation, T-cell proliferation, etc.). Exemplary methods are described in WO 97/28259; WO 98/16247; WO 99/11275, WO 98/55495, and WO 00/61151, as well as U.S. Pat. Nos. 7,745,606, 8,158,768, and 8,871,732 to Dynavax Technologies Corporation. Accordingly, these and other methods can be used to detect and quantify immunomodulatory activity of CpG oligonucleotides.

CpG oligonucleotides may contain modifications. Suitable modifications include but are not limited to, modifications of the 3′OH or 5′OH group, modifications of the nucleotide base, modifications of the sugar component, and modifications of the phosphate group. Modified bases may be included in the palindromic sequence as long as the modified base(s) maintains the same specificity for its natural complement through Watson-Crick base pairing (e.g., the palindromic portion of the CpG-C oligonucleotide remains self-complementary).

CpG oligonucleotides may be linear, may be circular or include circular portions and/or a hairpin loop. CpG-C oligonucleotides may be single stranded or double stranded. CpG oligonucleotides may be DNA, RNA, or a DNA/RNA hybrid.

CpG oligonucleotides may contain naturally-occurring or modified, non-naturally occurring bases, and may contain modified sugar, phosphate, and/or termini. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. In some embodiments, CpG oligonucleotides have only phosphorothioate linkages, only phosphodiester linkages, or a combination of phosphodiester and phosphorothioate linkages.

Sugar modifications known in the field, such as 2′-alkoxy-RNA analogs, 2′-amino-RNA analogs, 2′-fluoro-DNA, and 2′-alkoxy- or amino-RNA/DNA chimeras and others described herein, may also be made and combined with any phosphate modification. Examples of base modifications include, but are not limited to, addition of an electron-withdrawing moiety to C-5 and/or C-6 of a cytosine of the CpG oligonucleotide (e.g., 5-bromocytosine, 5-chlorocytosine, 5-fluorocytosine, 5-iodocytosine) and C-5 and/or C-6 of a uracil of the CpG oligonucleotide (e.g., 5-bromouracil, 5-chlorouracil, 5-fluorouracil, 5-iodouracil). As noted above, use of a base modification in a palindromic sequence of a CpG oligonucleotide should not interfere with the self-complementarity of the bases involved for Watson-Crick base pairing. However, outside of a palindromic sequence, modified bases may be used without this restriction. For instance, 2′-O-methyl-uridine and 2′-O-methyl-cytidine may be used outside of the palindromic sequence, whereas, 5-bromo-2′-deoxycytidine may be used both inside and outside the palindromic sequence. Other modified nucleotides, which may be employed both inside and outside of the palindromic sequence include 7-deaza-8-aza-dG, 2-amino-dA, and 2-thio-dT.

Duplex (i.e., double stranded) and hairpin forms of most oligonucleotides are in dynamic equilibrium, with the hairpin form generally favored at low oligonucleotide concentration and higher temperatures. Covalent interstrand or intrastrand cross-links increase duplex or hairpin stability, respectively, towards thermal-, ionic-, pH-, and concentration-induced conformational changes. Chemical cross-links can be used to lock the polynucleotide into either the duplex or the hairpin form for physicochemical and biological characterization. Cross-linked oligonucleotides that are conformationally homogeneous and are “locked” in their most active form (either duplex or hairpin form) could potentially be more active than their uncross-linked counterparts. Accordingly, some CpG oligonucleotides of the present disclosure contain covalent interstrand and/or intrastrand cross-links.

The techniques for making polynucleotides and modified polynucleotides are known in the art. Naturally occurring DNA or RNA, containing phosphodiester linkages, are generally synthesized by sequentially coupling the appropriate nucleoside phosphoramidite to the 5′-hydroxy group of the growing oligonucleotide attached to a solid support at the 3′-end, followed by oxidation of the intermediate phosphite triester to a phosphate triester. Once the desired polynucleotide sequence has been synthesized, the polynucleotide is removed from the support, the phosphate triester groups are deprotected to phosphate diesters and the nucleoside bases are deprotected using aqueous ammonia or other bases (see, e.g., Beaucage “Oligodeoxyribonucleotide Synthesis” in Protocols for Oligonucleotides and Analogs, Synthesis and Properties (Agrawal, ed.) Humana Press, Totowa, N.J., 1993; Warner et al., DNA 3:401, 1984; and U.S. Pat. No. 4,458,066).

The CpG oligonucleotide may contain phosphate-modified oligonucleotides, some of which are known to stabilize the oligonucleotide. Accordingly, some embodiments include stabilized CpG oligonucleotides. Synthesis of oligonucleotides containing modified phosphate linkages or non-phosphate linkages are also known in the art (see, e.g., Matteucci “Oligonucleotide Analogs: an Overview” in Oligonucleotides as Therapeutic Agents, (D. J. Chadwick and G. Cardew, ed.) John Wiley and Sons, New York, N.Y., 1997). The phosphorous derivative (or modified phosphate group), which can be attached to the sugar or sugar analog moiety in the oligonucleotide, can be a monophosphate, diphosphate, triphosphate, alkylphosphonate, phosphorothioate, phosphorodithioate, phosphoramidate, or the like. The preparation of the above-noted phosphate analogs, and their incorporation into nucleotides, modified nucleotides and oligonucleotides, per se, has already been well described (see, e.g., Peyrottes et al., Nucleic Acids Res, 24:1841-1848, 1996; Chaturvedi et al., Nucleic Acids Res, 24:2318-2323, 1996; and Schultz et al., Nucleic Acids Res, 24:2966-2973, 1996). For example, synthesis of phosphorothioate oligonucleotides is similar to that described above for naturally occurring oligonucleotides except that the oxidation step is replaced by a sulfurization step (see, e.g., Zon “Oligonucleoside Phosphorothioates” in Protocols for Oligonucleotides and Analogs, Synthesis and Properties (Agrawal, ed.) Humana Press, pp. 165-190, 1993).

CpG oligonucleotides can comprise one or more ribonucleotides (containing ribose as the only or principal sugar component), deoxyribonucleotides (containing deoxyribose as the principal sugar component), modified sugars, or sugar analogs. Thus, in addition to ribose and deoxyribose, the sugar moiety can be pentose, deoxypentose, hexose, deoxyhexose, glucose, arabinose, xylose, lyxose, and a sugar analog cyclopentyl group. The sugar can be in pyranosyl or in a furanosyl form. In the CpG-C oligonucleotide, the sugar moiety is the furanoside of ribose, deoxyribose, arabinose or 2′-O-alkylribose, and the sugar can be attached to the respective heterocyclic bases in either anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification in the CpG-C oligonucleotide includes, but is not limited to, 2′-O-methyl-uridine and 2′-O-methyl-cytidine. The preparation of these sugars or sugar analogs and the respective nucleosides wherein such sugars or analogs are attached to a heterocyclic base (nucleic acid base) per se is known, and therefore need not be described in further detail herein. Sugar modifications may also be made and combined with any phosphate modification in the preparation of a CpG oligonucleotide.

The heterocyclic bases, or nucleic acid bases, which are incorporated in the CpG oligonucleotide can be the naturally-occurring principal purine and pyrimidine bases, (namely uracil, thymine, cytosine, adenine, and guanine, as mentioned above), as well as naturally-occurring and synthetic modifications of said principal bases. Thus, a CpG oligonucleotide may include one or more of inosine, 2′-deoxyuridine, and 2-amino-2′-deoxyadenosine.

### 6. Pharmaceutical Compositions and Methods of Administration

The Cbl-b inhibitor compounds and second agents provided herein can be formulated into pharmaceutical compositions using methods available in the art and those disclosed herein. In particular embodiments, the Cbl-b inhibitor compound is formulated in a pharmaceutical composition comprising the compound and one or more pharmaceutically acceptable carriers, diluents, or excipients. In certain embodiments, the second agent is formulated according to the formulations known in the art for the inhibitor. In particular embodiments, the Cbl-b inhibitor compound is formulated in a pharmaceutical composition suitable for oral administration. In particular embodiments, the second agent is formulated in a pharmaceutical composition suitable for parenteral administration. While the Cbl-b inhibitor compound and second agent are not expected to be formulated in the same composition, this embodiment is not excluded from the description herein.

The methods provided herein encompass administering pharmaceutical compositions comprising a Cbl-b compound or a second agent and one or more compatible and pharmaceutically acceptable carriers. In this context, the term “pharmaceutically acceptable” means approved by a regulatory agency of the Federal or State government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil, and the like. Water can be used as a carrier when the pharmaceutical composition is administered intravenously. Saline solutions and aqueous dextrose and glycerol solutions can also be employed as liquid carriers, particularly for injectable solutions. Examples of suitable pharmaceutical carriers are described in Martin, E., Remington's Pharmaceutical Sciences.

In clinical practice the pharmaceutical compositions provided herein may be administered by any route known in the art. Exemplary routes of administration include, but are not limited to, inhalation, intraarterial, intradermal, intramuscular, intraperitoneal, intravenous, nasal, parenteral, pulmonary, oral, and subcutaneous routes. In certain embodiments, a pharmaceutical composition provided herein is administered parenterally.

The compositions for parenteral administration can be emulsions or sterile solutions. Parenteral compositions may include, for example, propylene glycol, polyethylene glycol, vegetable oils, and injectable organic esters (e.g., ethyl oleate). These compositions can also contain wetting, isotonizing, emulsifying, dispersing, and stabilizing agents. Sterilization can be carried out in several ways, for example, using a bacteriological filter, by radiation, or by heating. Parenteral compositions can also be prepared in the form of sterile solid compositions which can be dissolved at the time of use in sterile water or any other injectable sterile medium.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic agents (e.g., as described herein).

The pharmaceutical composition may comprise one or more pharmaceutical excipients. Any suitable pharmaceutical excipient may be used, and one of ordinary skill in the art is capable of selecting suitable pharmaceutical excipients. Non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene glycol, glycol, water, ethanol, and the like. Whether a particular excipient is suitable for incorporation into a pharmaceutical composition or dosage form depends on a variety of factors well known in the art including, but not limited to, the way in which the dosage form will be administered to a subject and the specific compound, agent, and/or antibody in the dosage form. The composition or single unit dosage form, if desired, can also contain minor amounts of wetting or emulsifying agents, or pH buffering agents. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference herein in its entirety.

In certain embodiments, the pharmaceutical composition comprises an anti-foaming agent. Any suitable anti-foaming agent may be used. In certain embodiments, the anti-foaming agent is selected from an alcohol, an ether, an oil, a wax, a silicone, a surfactant, and combinations thereof. In certain embodiments, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In certain embodiments, the pharmaceutical composition comprises a co-solvent. Illustrative examples of co-solvents include ethanol, poly(ethylene) glycol, butylene glycol, dimethylacetamide, glycerin, and propylene glycol.

In certain embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In certain embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In certain embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In certain embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference herein in its entirety.

In certain embodiments, the pharmaceutical composition comprises a solvent. In certain embodiments, the solvent is saline solution, such as a sterile isotonic saline solution or dextrose solution. In certain embodiments, the solvent is water for injection (WFI).

In certain embodiments, the pharmaceutical compositions are in a particulate form, such as a microparticle or a nanoparticle. Microparticles and nanoparticles may be formed from any suitable material, such as a polymer or a lipid. In certain embodiments, the microparticles or nanoparticles are micelles, liposomes, or polymersomes.

Further provided herein are anhydrous pharmaceutical compositions and dosage forms comprising therapeutic agent, since, in certain embodiments, water can facilitate the degradation of some antibodies.

Anhydrous pharmaceutical compositions and dosage forms provided herein can be prepared using anhydrous or low moisture containing ingredients and/or low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition can be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Lactose-free compositions provided herein can comprise excipients that are well known in the art and are listed, for example, in the U.S. Pharmocopia (USP) SP (XXI)/NF (XVI). In general, lactose-free compositions comprise an active ingredient, a binder/filler, and a lubricant in pharmaceutically compatible and pharmaceutically acceptable amounts. Exemplary lactose-free dosage forms comprise an active ingredient, microcrystalline cellulose, pre-gelatinized starch, and magnesium stearate.

Also provided are pharmaceutical compositions and dosage forms that comprise one or more excipients that reduce the rate by which a compound and/or an agent will decompose. Such excipients, which are referred to herein as “stabilizers,” include, but are not limited to, antioxidants such as ascorbic acid, pH buffers, or salt buffers.

In human therapeutics, the physician will determine the posology considered most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic compounds, agents, and/or antibodies as described herein.

The amounts of the Cbl-b compound or composition and second agent or composition which will be effective in the prevention or treatment of a disorder or one or more symptoms thereof will vary with the nature and severity of the disease or condition, and the route by which the compounds, agents, and/or compositions are administered. The frequency and dosage will also vary according to factors specific for each subject depending on the specific therapy (e.g., therapeutic or prophylactic compounds and/or agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the compound, agent, and/or antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram).

In certain embodiments, the dosage of the Cbl-b compound provided herein, based on mass of the subject, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the Cbl-b compound is 0.1 mg to 1000 mg, 0.1 mg to 900 mg, 0.1 mg to 800 mg, 0.1 mg to 750 mg, 0.1 mg to 700 mg, 0.1 mg to 600 mg, 0.1 mg to 500 mg, 0.1 mg to 400 mg, 0.1 mg to 300 mg, 0.1 mg to 250 mg, 0.1 mg to 200 mg, 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 mg to 15 mg, 0.5 mg to 12 mg, 0.5 mg to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

In certain embodiments, the dosage of the second agent is according to the product label or other instruction. In certain embodiments, the dosage of the PD-1 axis antagonist, based on weight of the antagonist, administered to prevent, treat, manage, or ameliorate a disorder, or one or more symptoms thereof in a subject is 0.1 mg/kg, 1 mg/kg, 2 mg/kg, 3 mg/kg, 4 mg/kg, 5 mg/kg, 6 mg/kg, 10 mg/kg, or 15 mg/kg or more of a subject's body weight. In another embodiment, the dosage of the PD-1 axis antagonist is 0.1 mg to 1000 mg, 0.1 mg to 900 mg, 0.1 mg to 800 mg, 0.1 mg to 750 mg, 0.1 mg to 700 mg, 0.1 mg to 600 mg, 0.1 mg to 500 mg, 0.1 mg to 400 mg, 0.1 mg to 300 mg, 0.1 mg to 250 mg, 0.1 mg to 200 mg, 0.1 mg to 200 mg, 0.1 mg to 100 mg, 0.1 mg to 50 mg, 0.1 mg to 25 mg, 0.1 mg to 20 mg, 0.1 mg to 15 mg, 0.1 mg to 10 mg, 0.1 mg to 7.5 mg, 0.1 mg to 5 mg, 0.1 to 2.5 mg, 0.25 mg to 20 mg, 0.25 to 15 mg, 0.25 to 12 mg, 0.25 to 10 mg, 0.25 mg to 7.5 mg, 0.25 mg to 5 mg, 0.25 mg to 2.5 mg, 0.5 mg to 20 mg, 0.5 to 15 mg, 0.5 to 12 mg, 0.5 to 10 mg, 0.5 mg to 7.5 mg, 0.5 mg to 5 mg, 0.5 mg to 2.5 mg, 1 mg to 20 mg, 1 mg to 15 mg, 1 mg to 12 mg, 1 mg to 10 mg, 1 mg to 7.5 mg, 1 mg to 5 mg, or 1 mg to 2.5 mg.

The dose of either agent can be administered according to a suitable schedule, for example, one time, two times, three times, or four times weekly. It may be necessary to use dosages of the agents outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat, or ameliorate such disorders, but insufficient to cause, or sufficient to reduce, adverse effects associated with the agents provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of a compound and/or an agent provided herein followed by one or more maintenance doses.

In certain embodiments, a dose of a compound and/or an agent provided herein can be administered to achieve a steady-state concentration of the treatment in blood or serum of the subject. The steady-state concentration can be determined by measurement according to techniques available to those of skill or can be based on the physical characteristics of the subject such as height, weight, and age.

In certain embodiments, administration of the same composition may be repeated and the administrations may be separated by at least one day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least one day, 2 days, 3 days, 5 days, 10 days, 15 days, 30 days, 45 days, 2 months, 75 days, 3 months, or 6 months.

### 7. Therapeutic Applications

The combinations provided herein may be useful for the treatment of any disease or condition involving abnormal cell growth or proliferation. In certain embodiments, the disease or condition is a disease or condition that can benefit from treatment with Cbl-b inhibitor compound or a PD-1 axis antagonist, or both; or Cbl-b compound and one or more immunostimulatory sequences; or Cbl-b compound and one or more TLR agonists. In certain embodiments, the disease or condition is a disease or condition that can benefit from treatment with Cbl-b inhibitor compound or a TLR9 agoinst, or both. In certain embodiments, the disease or condition is a cancer. In certain embodiments, the disease or condition is a solid tumor. In certain embodiments, the disease or condition is a hematological cancer.

Any suitable cancer may be treated with the combinations provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer (including triple-negative breast cancer, or TNBC), bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fallopian tube carcinoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer (NSCLC), oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, primary peritoneal carcinoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In certain embodiments, the disease to be treated with the combinations provided herein is gastric cancer, colorectal cancer, renal cell carcinoma, cervical cancer, non-small cell lung carcinoma, ovarian cancer, uterine cancer, fallopian tube carcinoma, primary peritoneal carcinoma, uterine corpus carcinoma, endometrial carcinoma, prostate cancer, breast cancer, head and neck cancer, brain carcinoma, liver cancer, pancreatic cancer, mesothelioma, and/or a cancer of epithelial origin. In particular embodiments, the disease is colorectal cancer. In certain embodiments, the disease is ovarian cancer. In certain embodiments, the disease is breast cancer. In certain embodiments, the disease is triple-negative breast cancer (TNBC). In certain embodiments, the disease is lung cancer. In certain embodiments, the disease is non-small cell lung cancer (NSCLC). In certain embodiments, the disease is head and neck cancer. In certain embodiments, the disease is renal cell carcinoma. In certain embodiments, the disease is brain carcinoma. In certain embodiments, the disease is endometrial cancer.

### 8. Kits

In certain embodiments, a compound or combination provided herein is provided in the form of a kit (i.e., a packaged combination of reagents in predetermined amounts with instructions for performing a procedure). In certain embodiments, the procedure is a therapeutic procedure. In certain embodiments, the kit comprises a Cbl-b inhibitor compound, or a composition thereof, and instructions for use in combination with a PD-1 axis antagonist, one or more immunostimulatory sequences, and/or one or more TLR agonists. In certain embodiments, the kit comprises a PD-1 axis antagonist, or a composition thereof, and instructions for use in combination with a Cbl-b inhibitor compound. In certain embodiments, the kit comprises a Cbl-b inhibitor compound, or a composition thereof, and a PD-1 axis antagonist, or composition thereof. In certain embodiments, the kit comprises a Cbl-b inhibitor compound, or a composition thereof, and instructions for use in combination with a TLR9 agonist. In certain embodiments, the kit comprises a TLR9 agonist, or a composition thereof, and instructions for use in combination with a Cbl-b inhibitor compound. In certain embodiments, the kit comprises a Cbl-b inhibitor compound, or a composition thereof, and a TLR9 agonist, or composition thereof. In certain embodiments, the kit comprises one or more immunostimulatory sequences, or a composition thereof, and instructions for use in combination with a Cbl-b inhibitor compound. In certain embodiments, the kit comprises a Cbl-b inhibitor compound, or a composition thereof, and one or more immunostimulatory sequences, or composition thereof.

### EXAMPLES

### Example 1

The present example provides assays and results for treating tumor models in vivo with single agent Cbl-b compounds described herein.

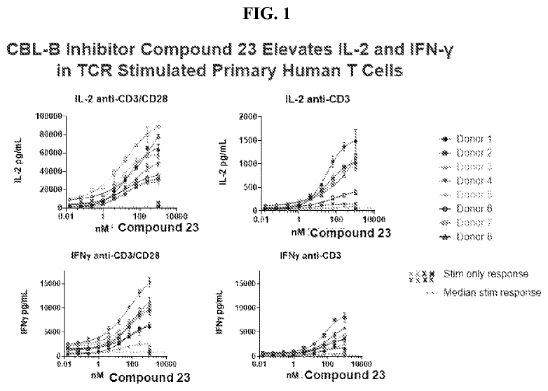

As shown in FIG. 1, the effects of Compound 23 on total primary human T-cells were assessed. Cells were stimulated with plate bound anti-CD3 (right) or anti-CD3/anti-CD28 (left) in presence or absence of the indicated concentration of Compound 23. Release of IL-2 or IFN-γ were assessed by ELISA. Compound 23 inhibition of Cbl-b enhanced IL-2 and IFN-γ secretion in primary human T cells stimulated with anti-CD3 antibodies, in both the presence and absence of CD28 co-stimulation, although to a lesser degree in the absence of co-stimulation.

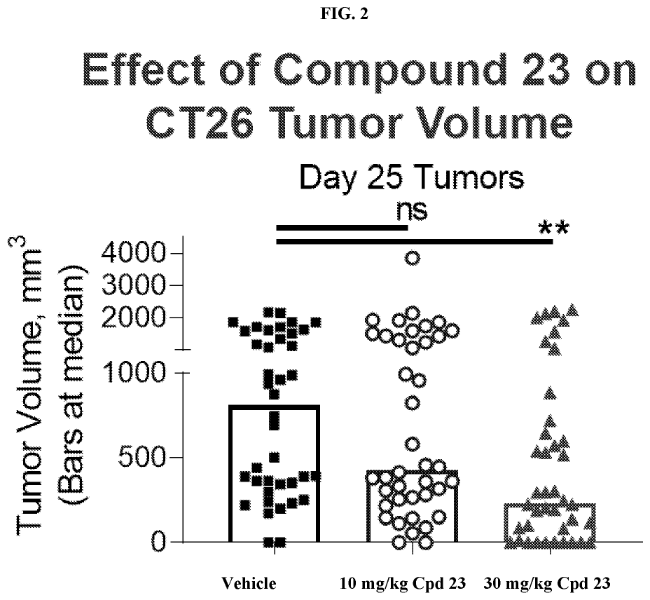

As shown in FIG. 2, the effects of Compound 23 on CT26 tumor volume in a mouse models were assessed. Mice bearing tumors on their left and right flanks were treated from Day 7 to Day 32 with oral Compound 23 at 10 mg/kg or 30 mg/kg or vehicle. Volumes at Day 25 are indicated. Statistics were calculated with one-way ANOVA and Dunn's multiple comparisons test ** P<0.01.

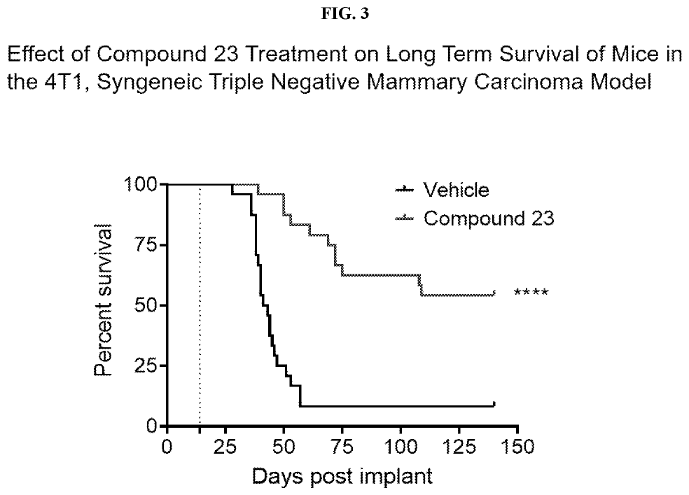

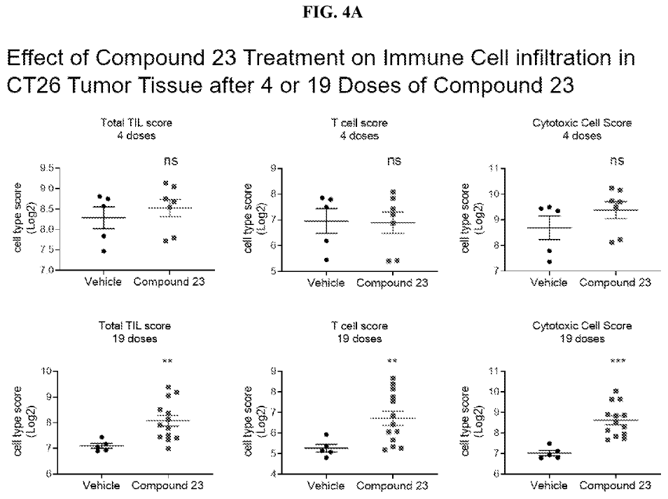

Another in vivo study evaluated the ability of Compound 23 treatment, started before the primary tumor resection, to eradicate 4T1 breast carcinoma metastatic disease. The 4T1 triple negative mammary carcinoma is a transplantable tumor cell line that is highly tumorigenic and invasive and, unlike most tumor models, spontaneously metastasizes from a primary tumor growing in the mammary gland to multiple distant sites including lymph nodes, blood, liver, lung, brain, and bone. Tumor cells are easily transplanted into the mammary glands so that the primary tumor grows in the anatomically relevant site. The progressive spread of 4T1 metastases to other organs is very similar to that of human mammary cancer. Animals die from disseminated metastatic disease regardless of resection of the primary tumor (Pulaski et al., 2000, Current Protocols in Immunology 39(1):20.2.1-20.2.16). As shown in FIG. 3, mice bearing 4T1 tumors in the 4th mammary fat pad on the ventral flank were treated from Day 8 to Day 46 by daily oral administration of either: vehicle (0.5% methylcellulose, 0.2% Polysorbate 80 in deionized water+1 equivalent HCl) or Compound 23 at 30 mg/kg daily. Beginning on Day 7, tumor volumes were measured twice weekly until they reached a mean tumor volume of 145 mm³ when the primary tumors were resected on Day 14 or 15 (dotted line). Median survival times were calculated for each group and body weights were monitored until the end of the study on Day 140. Body weights were measured twice weekly then once weekly starting Day 47. FIG. 3 shows percentage survival over time through Day 140. Statistical significance of differences in conditional survival between groups was evaluated using the Log-rank (Mantel-Cox) test. Significance was reported as not significant (ns) P>0.05, * P≤0.05, ** P≤0.01, *** P≤0.001, and **** P≤0.0001. Treatment with Compound 23 dosed orally at 30 mg/kg significantly increased median survival time compared to vehicle control. Median survival is reported as undefined for the Compound 23 group, as survival exceeds 50% at Day 140. Importantly, 54% of the mice treated with Compound 23 remained tumor free until the end of the study, suggesting that Compound 23 can significantly extend survival in a model of metastatic disease.

A shown in these experiments, in vivo oral administration of Compound 23 in mice demonstrated significant anti-tumor activity in a colon carcinoma tumor models, CT26, as well as a metastatic triple negative breast tumor model, 4T1.

### Example 2

In this example, the gene expression changes induced in tumors from mice treated with Compound 23 studies were as investigated. To perform this assessment, the nCounter PanCancer Immune Profiling Panel was used, which is a multiplexed gene expression panel developed by NanoString Technologies that measures expression of 770 immune and cancer related genes (Eastel et al., 2019, Expert Review of Molecular Diagnostics, 19(7):591-598). This technology is a unique gene expression tool that covers many important features of the immune response in the tumor microenvironment and was used to facilitate the understanding of immune related changes in tumors treated with Compound 23.

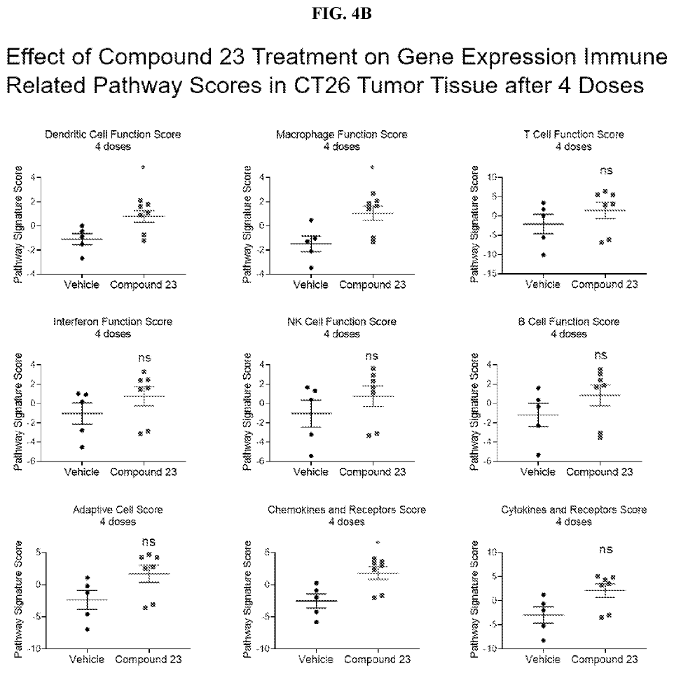

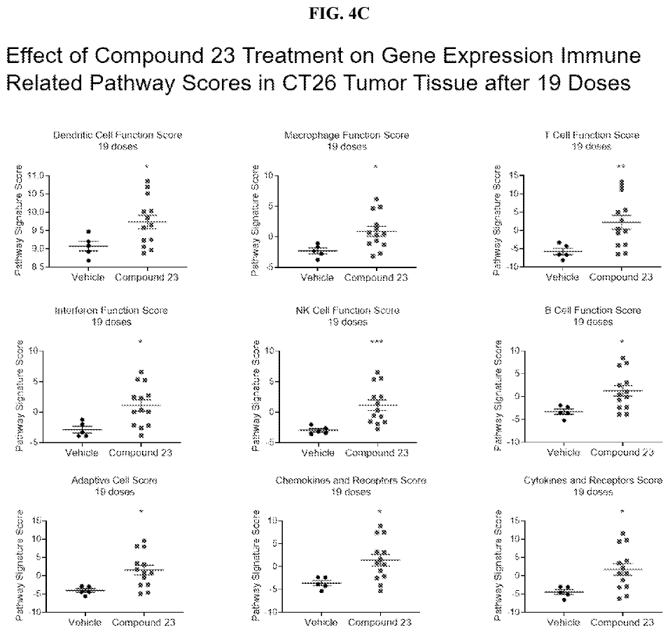

In FIG. 4A, CT26 tumors were harvested after 4 doses and after 19 doses of Compound 23 or vehicle and gene expression was directly measured using the nCounter Mouse PanCancer Immune Profiling Panel. Analysis was performed using the nSolver 4.0 and the nCounter Advanced Analysis software comparing gene expression in Compound 23 to vehicle treated tumors. FIG. 4A (top row) shows individual TIL, T-cell and cytotoxic cell scores between vehicle and Compound 23 treated groups after 4 doses. FIG. 4A (bottom row) shows individual TIL, T-cell and cytotoxic cell scores between vehicle and Compound 23 treated groups after 19 doses. Statistical significance of differences in mean cell type scores between Compound 23 and vehicle treated groups was evaluated using Mann-Whitney test (* P≤0.05, ** P≤0.01, and *** P≤0.001).

In FIG. 4B, CT26 tumors were harvested after 4 treatment doses and gene expression was directly measured using the nCounter Mouse PanCancer Immune Profiling Panel. Analysis was performed using the nSolver 4.0 and the nCounter Advanced Analysis software comparing gene expression in Compound 23 to vehicle treated tumors. As indicated in the titles above each panel, scatter plots of individual Pathway Signature Scores of vehicle and Compound 23 treated tumors are shown. Statistical significance of differences in Pathway Signature Scores between Compound 23 and vehicle treated groups was evaluated using Mann-Whitney test (* P≤0.05, ** P≤0.01, and *** P≤0.001).

In FIG. 4C, CT26 tumors were harvested after 19 treatment doses and gene expression was directly measured using the nCounter Mouse PanCancer Immune Profiling Panel. Analysis was performed using the nSolver 4.0 and the nCounter Advanced Analysis software comparing gene expression in Compound 23 to vehicle treated tumors. As indicated in the titles above each panel, scatter plots of individual Pathway Signature Scores of vehicle and Compound 23 treated tumors are shown. Statistical significance of differences in Pathway Signature Scores between Compound 23 and vehicle treated groups was evaluated using Mann-Whitney test (* P≤0.05, ** P≤0.01, and *** P≤0.001).

The immune context of tumors from Compound 23 treated mice were significantly changed compared to tumors from vehicle treated mice. After 4 doses, single agent oral Compound 23 administered daily at 30 mg/kg resulted in a clear trend of increased infiltration of tumor infiltrating lymphocytes (TIL) (P=0.6679), T-cell (P=0.7551), and cytotoxic cells (P=0.1061) (FIG. 4A). At the later time point, following 19 doses of oral Compound 23, there were significant increases in TIL (P=0.0044), T-cell (P=0.0098), and cytotoxic cell (P=0.0002) infiltration in CT26 tumors relative to vehicle treated animals (FIG. 4B).

After 4 doses of Compound 23, tumors exhibited significant gene expression changes in pathways associated with innate immune signaling, including dendritic cell function and macrophage function and chemokine receptors and pathways (FIG. 4C). Although there was a trend for increases in T-cell, B cell, NK cell and adaptive immune function, these changes were not significant. However, after 19 doses, Compound 23 treated CT26 tumors exhibited significantly enhanced function of immune related pathway scores, including ones related to dendritic cell function, macrophage function, T-cell function, NK cell function, B cell function as well as interferon function, adaptive immunity, chemokine and receptor response, and cytokine and receptor signature response (FIG. 4C). Collectively, these data demonstrate that Compound 23 induced anti-tumor activity in the CT26 syngeneic tumor model, enhancing both intratumoral density and function of immune cells.

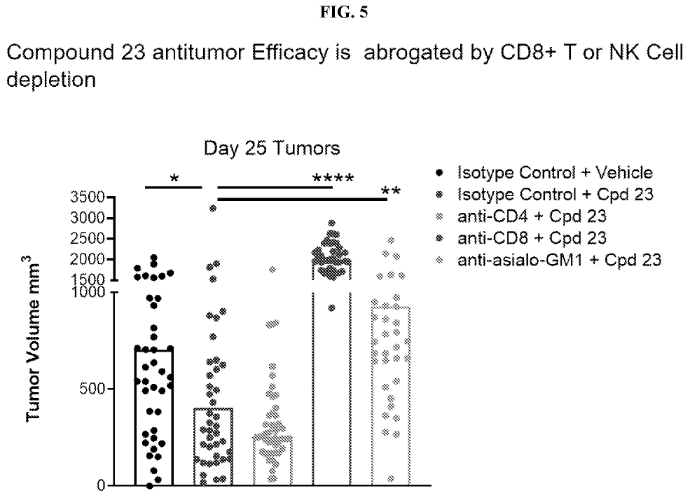

As shown in FIG. 5, mice bearing CT26 tumors on their left and right flanks were treated from Day 9 to Day 25 with oral Compound 23 at 30 mg/kg, PO QD, in the presence of depleting antibodies for CD4+ cells, CD8+× cells, or NK cells (anti-asialo-GM1). Tumor volume at Day 25 is indicated. CD8+ T-cell or NK cell depletion abrogates Compound 23 activity. Stats calculated with Mann-Whitney (Vehicle vs. Compound 23) or one-way ANOVA with Dunn's multiple comparisons test (Compound 23 vs. Depletion groups); * P≤0.05, ** P≤0.01, **** P≤0.0001 The efficacy of Compound 23 on tumors is linked to CD8+ T function and on NK cell function.

### Example 3

The present example provides assays and results for the effects of Cbl-b compound treatment on immune cell phenotypes in mouse tumor models and correlation of treatment efficacy with immune cell levels.

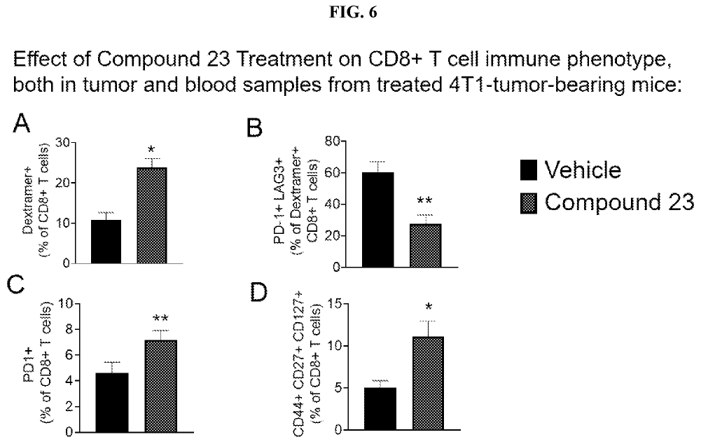

In FIG. 6, mice bearing orthotopic 4T1 tumors in their 4th mammary fat pad on the left ventral flank were treated from Day 9 to Day 28 by administration of either: vehicle control (0.5% methylcellulose, 0.2% Polysorbate 80 in deionized water+1 equivalent HCl) given daily PO; or Compound 23 at 30 mg/kg given daily PO. Tumor and blood samples were harvested on Day 28 and analyzed by Flow Cytometry.

FIG. 6A shows that Compound 23 increased the frequency of tumor gp70 antigen-specific CD8+ T-cells (AH1 Dextramer+ CD8+ T-cells) in 4T1 tumors from treated mice. FIG. 6B shows that Compound 23 decreased the frequency of tumor gp70 antigen-specific CD8+ T-cells (AH1 Dextramer+ CD8+ T-cells) with exhaustion phenotype (PD-1+ LAG3+) in 4T1 tumors from treated mice. FIG. 6C shows that Compound 23 increased the frequency of circulating CD8+ T-cells with activated phenotype (PD-1+) in the blood of treated 4T1-tumor-bearing mice. FIG. 6D shows that Compound 23 increased the frequency of circulating CD8+ T-cells with memory phenotype (CD44+CD27+CD127+) in the blood of treated 4T1-tumor-bearing mice.

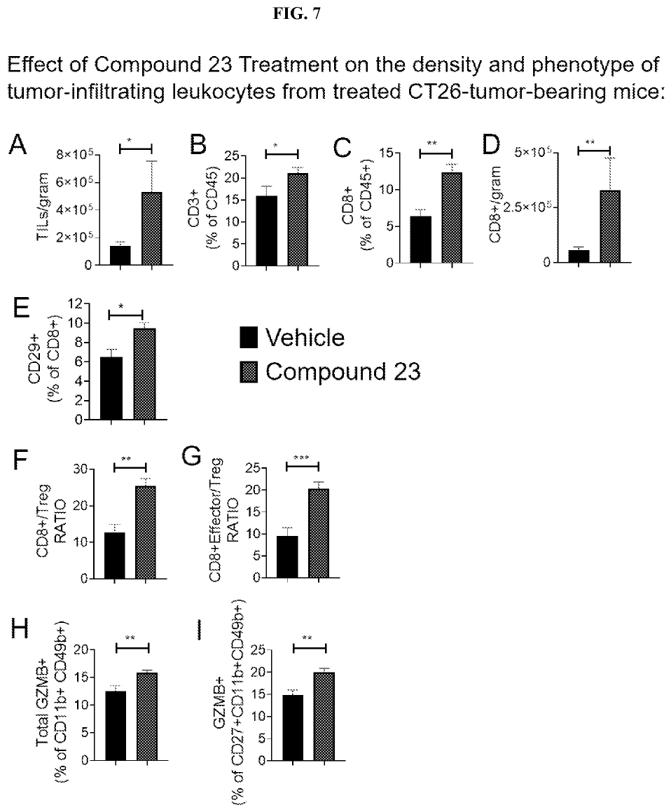

In FIG. 7, mice bearing CT26 tumors on their left and right flanks were treated from Day 10 to Day 28 by administration of either vehicle (0.5% methylcellulose, 0.2% Polysorbate 80 in deionized water+1 equivalent HCl) daily or orally (PO) Compound 23 at 30 mg/kg daily. CT26 tumors were harvested after 19 doses of Compound 23 or vehicle and tumor-infiltrating immune cell density and phenotype was assessed by Flow Cytometry.

FIG. 7A shows that Compound 23 increased the number of tumor-infiltrating leukocytes (TIL) per gram of tumor in CT26 tumors from treated mice. FIG. 7B shows that Compound 23 increased the frequency of total CD3+ T cells as a percentage of CD45+ leukocytes in CT26 tumors from treated mice. FIGS. 7C and 7D show that Compound 23 increased the frequency of total CD8+ T cells as a percentage of CD45+ leukocytes and the number of total CD8+ T-cells per gram of tumor in CT26 tumors from treated mice. FIG. 7E shows that Compound 23 increased the frequency of CD8+ T-cells that express the activation marker CD29 (CD29+) in CT26 tumors from treated mice. FIGS. 7F and 7G show that Compound 23 increased the CD8+ T-cells to Tregs ratio and CD8+ effector T-cells (identified as PD1+) to Tregs ratio in CT26 tumors from treated mice. FIGS. 7H and 7I show that Compound 23 increased the frequency of Tumor-infiltrating NK cells with cytotoxic phenotype in CT26 tumors from treated mice. FIG. 7H shows the frequency of activated NK cells (CD11b+) that express Granzyme+, and FIG. 7I shows the frequency of activated NK cells (CD27+CD11 b+) that express Granzyme+ in CT26 tumors from Compound 23 treated mice.

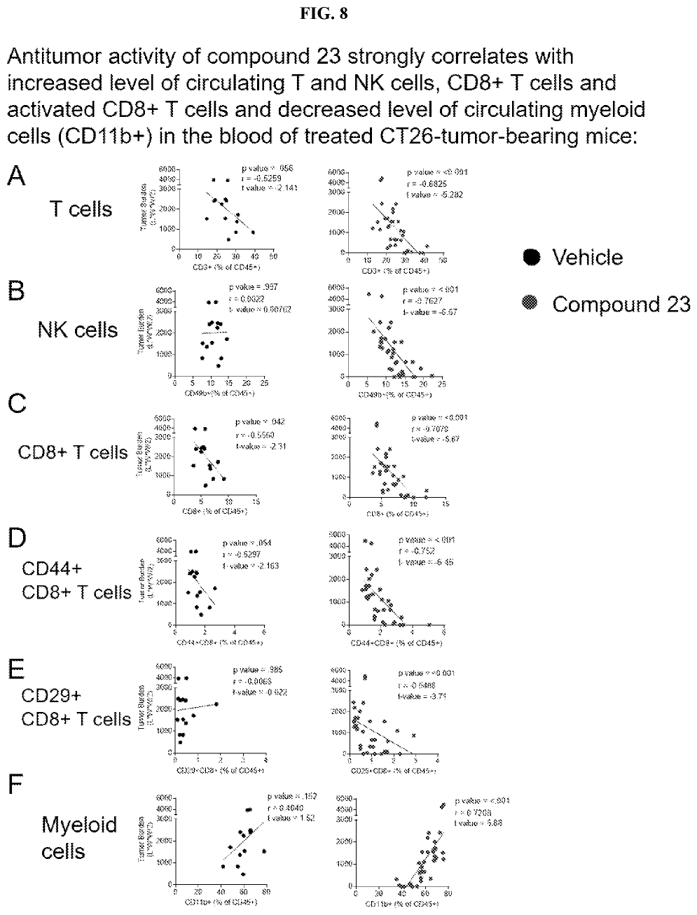

In FIG. 8, mice bearing CT26 tumors on their left and right flanks were treated from Day 10 to Day 28 by administration of either vehicle (0.5% methylcellulose, 0.2% Polysorbate 80 in deionized water+1 equivalent HCl) daily or orally (PO) Compound 23 at 30 mg/kg daily. CT26 tumors were harvested after 19 doses of Compound 23 or vehicle and frequency and phenotype of immune cells was assessed in the blood by Flow Cytometry. The Spearman correlation test was applied to calculate the correlation between levels of circulating immune cells and the tumor volume at Day 28.

Tumor regression in response to Compound 23 treatments correlated strongly with elevated frequency of total circulating T-cells (FIG. 8A) and NK cells (FIG. 8B) in the blood of treated CT26-tumor-bearing mice. Similar positive correlations were observed between tumor volume and the frequency of circulating CD8+ T-cells (FIG. 8C) and activated CD8+ T cells expressing the activation markers CD44 (FIG. 8D) and CD29 (FIG. 8E). In contrast, increased tumor growth inhibition correlated with decreased levels of circulating myeloid cells, characterized by the expression of the CD11b marker (FIG. 8F).

### Example 4

This example provides assays and results for treating tumor models in vivo with combinations of Cbl-b compounds and TLR9 agonists as described herein. The TLR9 agonist is a CpG oligonucleotide according to SEQ ID NO:10 with a phosphorothioate backbone.

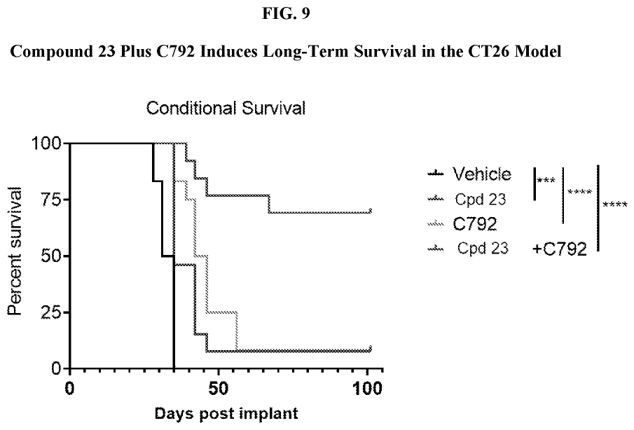

FIG. 9 provides results in a tumor model. With the CT26 Model, mice bearing CT26 tumors on their left and right flanks were treated from Day 12 by administration of either: vehicle (0.5% methylcellulose, 0.2% Polysorbate 80 in deionized water+1 equivalent HCl) daily, orally (PO); Compound 23 at 30 mg/kg daily, PO; SEQ ID NO:10 at 50 μg once weekly by intraperitoneal (IP) injection; or the combination of Compound 23 at 30 mg/kg daily, PO and SEQ ID NO:10 at 50 μg once weekly by IP injection.

FIG. 9 shows percentage survival overtime through Day 100. Statistical significance of differences in conditional survival between groups was evaluated using the Log-rank (Mantel-Cox) test. Significance was reported as not significant (ns)P>0.05, * P≤0.05, ** P≤0.01, *** P≤0.001, and **** P≤0.0001. None of the vehicle mice survived past Day 35. Mice treated with the combination of Compound 23 at 30 mg/kg daily, PO and SEQ ID NO:10 at 50 μg once weekly by IP injection survived longer and in higher numbers compared to the individual agents alone.

### EQUIVALENTS

The disclosure set forth above may encompass multiple distinct embodiments with independent utility. Although each of these embodiments has been disclosed in its particular form(s), the specific embodiments thereof as disclosed and illustrated herein are not to be considered in a limiting sense, because numerous variations are possible. The subject matter disclosed herein includes all novel and nonobvious combinations and subcombinations of the various elements, features, functions, and/or properties disclosed herein. The following claims particularly point out certain combinations and subcombinations regarded as novel and nonobvious. Subject matter embodied in other combinations and subcombinations of features, functions, elements, and/or properties may be claimed in this patent application, in patent applications claiming priority to or from this patent application, or in related patent applications. Such claims, whether directed to a different embodiment or to the same embodiment, and whether broader, narrower, equal, or different in scope in comparison to the original claims, also are regarded as included within the subject matter of this disclosure.

One or more features from any embodiments described herein or in the figures may be combined with one or more features of any other embodiments described herein or in the figures without departing from the scope of the appended claims.

All publications, patents, and published patent applications cited in this specification are herein incorporated by reference as if each individual publication, patent, or published patent application were specifically and individually indicated to be incorporated by reference. Although the foregoing embodiments have been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A method of treating or preventing a disease or condition in a subject in need thereof, comprising administering to the subject:
(a) an effective amount of a Cbl-b inhibitor compound; and
(b) one or more immunostimulatory sequences.

2. The method of claim 1, wherein the immunostimulatory sequence is a CpG-C type oligonucleotide.

3. The method of claim 1, wherein the immunostimulatory sequence is a TLR7 agonist.

4. The method of claim 1, wherein the immunostimulatory sequence is a TLR9 agonist.

5. A method of treating or preventing a disease or condition in a subject in need thereof, comprising administering to the subject:
(a) an effective amount of a Cbl-b inhibitor compound; and
(b) one or more TLR agonists.

6. The method of claim 5, wherein the TLR agonist is a TLR7 agonist.

7. The method of claim 5, wherein the TLR agonist is resiquimod, imiquimod, or gardiquimod.

8. The method of claim 5, wherein the TLR agonist is a TLR9 agonist.

9. The method of claim 5, wherein the TLR agonist is lefitolimod, tilsotolimod, or a CpG oligonucleotide

10. The method of claim 5, wherein the TLR agonist is an oligodeoxynucleotide 12 to 100 bases in length comprising the sequence 5′-Nₓ(TCG(N_{q}))_{y}N_{w}(X¹X²CGX^{2′}X¹′(CG)ₚ)_{z}Nᵥ-3′ (SEQ ID NO:1), wherein each N is a nucleoside; x is 0, 1, 2, or 3; y is 1, 2, 3, or 4; w is 0, 1, or 2; p is 0 or 1; q is 0, 1, or 2; v is an integer from 0 to 89; and z is an integer from 1 to 20; X¹ and X¹′ are self-complementary deoxynucleosides; X² and X²′ are self-complementary deoxynucleosides; the 5′-T of (TCG(N_{q})_{y} is 0-3 bases from the 5′ end of the oligodeoxynucleotide; wherein the oligodeoxynucleotide comprises a palindromic sequence at least 8 bases in length including the first X¹X²CGX^{2′}X¹′(CG)ₚ (SEQ ID NO:2) of (X¹X²CGX^{2′}X¹′(CG)ₚ)_{z} (SEQ ID NO:3).

11. The method of claim 10, wherein x is 0; y is 1; w is 0; p is 0 or 1; q is 0, 1, or 2; v is an integer from 0 to 20; and z is 1, 2, 3, or 4.

12. The method of claim 10, wherein the oligodeoxynucleotide consists of 5′-TCGN_{q}(X¹X²CGX^{2′}X¹′CG)_{z}Nᵥ-3′ (SEQ ID NO:4) wherein q is 1, 2, 3, 4, or 5; v is an integer from 0 to 20; and z is 1,2,3, or 4.

13. The method of claim 10, wherein the oligodeoxynucleotide consists of 5′-TCGN_{q}TTCGAACGTTCGAACGTTNₛ-3′ (SEQ ID NO:5) wherein q is 1, 2, 3, 4, or 5; and s is an integer from 0 to 20.

14. The method of claim 10, wherein the oligodeoxynucleotide consists of a sequence selected from the group consisting of SEQ ID NOS:6-17.

15. The method of claim 10, wherein the oligodeoxynucleotide consists of a sequence according to SEQ ID NO:10.

16. The method of any one of claims 10-15, wherein the oligodeoxynucleotide comprises at least one phosphorothioate.

17. The method of any one of claims 10-15, wherein the oligodeoxynucleotide comprises a phosphorothioate backbone.

18. The method of any one of the previous claims, wherein the Cbl-b inhibitor compound is according to Formula (I), or a pharmaceutically acceptable stereoisomer, tautomer, salt, or solvate thereof 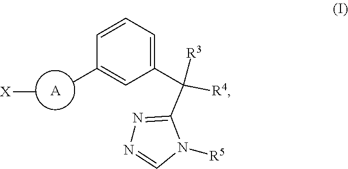
wherein 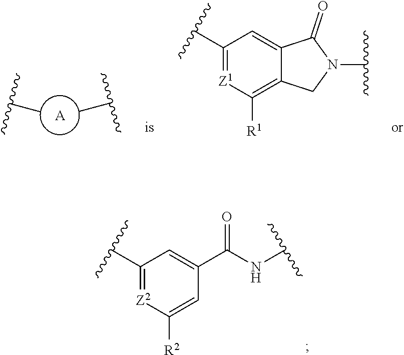
Z¹ is CH or nitrogen;
Z² is CH or nitrogen;
R¹ is —CF₃ or cyclopropyl;
R² is —CF₃ or cyclopropyl;
R³ is hydrogen, C₁-C₂ alkyl, or C₁-C₂ haloalkyl;
R⁴ is hydrogen, C₁-C₆ alkyl, C₁-C₆ haloalkyl, 4- to 8-membered heterocyclyl, or C₃-C₆ cycloalkyl, wherein the heterocyclyl or cycloalkyl groups are optionally substituted by one to five R⁶ groups;
or R³ and R⁴ are taken together with the carbon atom to which they are attached to form a spiro C₃-C₅ cycloalkyl or a spiro 4- to 6-membered heterocyclyl, each of which is optionally substituted by one to five R⁶ groups;
R⁵ is hydrogen, C₁-C₆ alkyl, C₁-C₆ haloalkyl, or C₃-C₆ cycloalkyl;
each R⁶ is independently C₁-C₆ alkyl, halo, hydroxy, —O(C₁-C₆ alkyl), —CN, C₁-C₆ alkyl-CN, C₁-C₆ alkyl-OH, or C₁-C₆ haloalkyl;
or two R⁶ groups attached to the same carbon atom are taken together with the carbon atom to which they are attached to form a spiro C₃-C₆ cycloalkyl or a spiro 4- to 6-membered heterocyclyl;
X is hydrogen, C₁-C₆ alkyl, C₁-C₆ haloalkyl, C₁-C₆ alkyl-OH, C₁-C₆ alkyl-CN,
C₃-C₆ cycloalkyl optionally substituted by one to five R⁸ groups, or 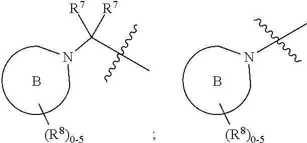
is a 4- to 7-membered heterocyclyl or a 5- to 8-membered heteroaryl, wherein each heterocyclyl or heteroaryl optionally contains one to two additional heteroatoms selected from the group consisting of nitrogen, oxygen, and sulfur, and wherein each heterocyclyl or heteroaryl is optionally substituted by one to five R⁸ groups;
each R⁷ is independently hydrogen, C₁-C₆ alkyl, C₁-C₆ alkyl-OH, or C₁-C₆ haloalkyl;
or two R⁷ groups are taken together with the carbon atom to which they are attached to form a spiro C₃-C₅ cycloalkyl or a spiro 3- to 5-membered heterocyclyl; and
each R⁸ is independently halo, C₁-C₆ alkyl, C₁-C₆ alkyl-CN, C₁-C₆ alkyl-OH, C₁-C₆ haloalkyl, —CN, oxo, or —O(C₁-C₆ alkyl);
or two R⁸ groups are taken together with the carbon atom or atoms to which they are attached to form a spiro or fused C₃-C₅ cycloalkyl or a 3- to 5-membered heterocyclyl.

19. The method of any one of the previous claims, wherein the Cbl-b inhibitor compound is selected from the compounds in Table 1, or a stereoisomer or tautomer thereof, or a pharmaceutically acceptable salt thereof.

20. The method of any one of the previous claims, wherein the disease or condition is a cancer.

21. The method of any one of the previous claims, wherein the disease or condition is a solid tumor.

22. The method of any one of the previous claims, wherein the disease or condition is a hematological cancer.
